(19) United States
(12) Patent Application Publication
(10) Pub. No.: US 2023/0277759 A1
(43) Pub. Date: Sep. 7, 2023

(54) Systems, Methods, and Apparatuses for Medical Agent Administration

(71) Applicant: DEKA Products Limited Partnership, Manchester, NH (US)

(72) Inventors: Dean Kamen, Bedford, NH (US); Richard J. Lanigan, Concord, NH (US); Eitan C. Zeira, Hollis, NH (US); Lee A. Batchelder, Auburn, NH (US); Hans E. Johnson, Salem, NH (US)

(21) Appl. No.: 18/178,211

(22) Filed: Mar. 3, 2023

Related U.S. Application Data
(60) Provisional application No. 63/316,336, filed on Mar. 3, 2022.

(51) Int. Cl. A61M 5/142

(52) U.S. Cl. CPC A61M 5/142; A61M 2039/1072

(57) ABSTRACT

A medical agent administration device may comprise a main body including a central region and a peripheral region. The peripheral region may have a plurality of petal members extending outwardly from the central region. The device may further comprise at least one coupling on a first face of the central region. The device may further comprise at least one sharp bearing body on an opposing face of the central region. Each of the at least one sharp bearing body may be in fluid communication with a respective one of the at least one coupling.

### CROSS-REFERENCE TO RELATED APPLICATIONS

The present application claims the benefit of U.S. Provisional Application Ser. No. 63/316,336, entitled Systems, Methods, and Apparatuses for Medical Agent Administration, filed Mar. 3, 2022, Attorney Docket Number 00101.00330.AA771.

### BACKGROUND

### Field of Disclosure

This disclosure relates to medical agent administration. More specifically, this disclosure relates to medical agent administration sets and other components which may aid in supporting administration of agent to a patient.

### SUMMARY

In accordance with an example embodiment of the present disclosure an example medical agent administration device may comprise a main body including a central region and a peripheral region. The peripheral region may have a plurality of petal members extending outwardly from the central region. The device may further comprise at least one coupling on a first face of the central region. The device may further comprise at least one sharp bearing body on an opposing face of the central region. Each of the at least one sharp bearing body may be in fluid communication with a respective one of the at least one coupling.

In some embodiments, the central region may be raised with respect to the peripheral region. In some embodiments, each of the at least one coupling may be a fitting. In some embodiments, the at least one coupling may include connector receivers each having a ramped face and a ledge face. In some embodiments, the at least one coupling may include at least one guide. In some embodiments, each of the at least one coupling may include a luer fitting. In some embodiments, the main body and coupling may be configured for injection molding with no side action. In some embodiments, the opposing face of the central region may include at least one rocker member. In some embodiments, the at least one sharp bearing body may be constructed of etched silicon. In some embodiments, each of the at least one sharp bearing body may be coupled to a respective one of an at least one stage projection on the opposing face in an injection molding operation. In some embodiments, each of the at least one sharp bearing body may be coupled to a respective one of an at least one stage projection on the opposing face via adhesive. In some embodiments, the device may further comprise a septum sealing a passage in fluid communication with the at least one sharp bearing body. In some embodiments, the opposing face of the central region may be at least partially covered with an adhesive bearing member. In some embodiments, the device may further comprise an adhesive bearing member. In some embodiments, each of the at least one sharp bearing body may include at least one microneedle. In some embodiments, each of the at least one sharp bearing body may include an array of microneedles. In some embodiments, a first of the at least one sharp bearing body may include at least one first microneedle having a first height. A second of the at least one sharp bearing body may include at least one second microneedle having a second height different from the first height. In some embodiments, the first height may be selected to place the at least one first microneedle in a shallow delivery destination and the second height may be selected to place the at least one second microneedle in a deeper delivery destination when the device is in use. In some embodiments, the shallow delivery destination may be an intradermal delivery destination. In some embodiments, the deeper delivery destination may be a subcutaneous delivery destination. In some embodiments, each of the at least one sharp bearing body may be in fluid communication with the respective one of the at least one coupling only.

In accordance with another example embodiment of the present disclosure and example medical agent administration device may comprise a main body including a central region and a peripheral region. The peripheral region may have a plurality of petal members extending outwardly from the central region. The device may further comprise a body coupled to the main body including at least one coupling on a first face of the central region and at least one sharp bearing body on an opposing face of the central region. Each of the at least one sharp bearing body may be in fluid communication with a respective one of the at least one coupling.

In some embodiments, the body may be coupled to a ridge included in a portion of the main body. In some embodiments, the body may include a plurality of tabs which couple into slits in the main body. In some embodiments, the central region may be raised with respect to the peripheral region. In some embodiments, each of the at least one coupling may be a fitting. In some embodiments, each of the at least one coupling may be a tubing receiver into which a fluid line leading to a fitting is coupled. In some embodiments, the at least one coupling may include connector receivers each having a ramped face and a ledge face. In some embodiments, the at least one coupling may include at least one guide. In some embodiments, each of the at least one coupling may include a luer fitting. In some embodiments, the main body and body may be injection molded. In some embodiments, the opposing face of the body may include at least one rocker member. In some embodiments, the at least one sharp bearing body may be constructed of etched silicon. In some embodiments, each of the at least one sharp bearing body may be coupled to a respective one of an at least one stage projection on the opposing face in an injection molding operation. In some embodiments, each of the at least one sharp bearing body may be coupled to a respective one of an at least one stage projection on the opposing face via adhesive. In some embodiments, the device may further comprise a septum sealing a passage in fluid communication with the at least one sharp bearing body. In some embodiments, the opposing face of the body may be at least partially covered with an adhesive bearing member. In some embodiments, the device may further comprise an adhesive bearing member. In some embodiments, each of the at least one sharp bearing body may include at least one microneedle. In some embodiments, each of the at least one sharp bearing body may include an array of microneedles. In some embodiments, a first of the at least one sharp bearing body may include at least one first microneedle having a first height. A second of the at least one sharp bearing body may include at least one second microneedle having a second height different from the first height. In some embodiments, the first height may be selected to place the at least one first microneedle in a shallow delivery destination and the second height may be selected to place the at least one second microneedle in a deeper delivery destination when the device is in use. In some embodiments, the shallow delivery destination may be an intradermal delivery destination. In some embodiments, the deeper delivery destination may be a subcutaneous delivery destination. In some embodiments, each of the at least one sharp bearing body may be in fluid communication with the respective one of the at least one coupling only.

In accordance with another example embodiments of the present disclosure an exemplary medical agent administration device may comprise a main body including a central region and a peripheral region. The peripheral region may have a plurality of petal members extending outwardly from the central region. The device may further comprise at least one coupling on a first face of the central region. The device may further comprise at least one sharp bearing body on an opposing face of the central region. Each of the at least one sharp bearing body may be in fluid communication with a respective one of the at least one coupling. The at least one sharp bearing body may be coupled to a stage projection on the opposing face.

In accordance with another example embodiment of the present disclosure an analyte sensor device may comprise a main body including a central region and a peripheral region. The peripheral region may include a plurality of petal members extending outwardly from the central region. The device may further comprise at least one sharp bearing body on a first face of the central region. Each of the at least one sharp bearing body may include at least one electrode. The device may include at least one first electrode and at least one second electrode associated with analyte sensing chemistry.

In some embodiments, the central region may be raised with respect to the peripheral region. In some embodiments, each of the at least one electrode may communicate with a conductive trace extending to an opposing side of the central region. In some embodiments, the device may include a first sharp bearing body including the at least one first electrode and a second sharp bearing body separate from the first sharp bearing body including the at least one second electrode. In some embodiments, an opposing face of the central region opposite the first face may include at least one coupling member for coupling the device to a transmitter. In some embodiments, each of the at least one sharp bearing body may be coupled to a stage projection on the first face of the central region. In some embodiments, each of the at least one sharp bearing body may be coupled to the stage projection in an injection molding operation. In some embodiments, the first face of the central region may include at least one stage projection. The at least one first and second electrodes each may be coupled to one of the at least one stage projection. In some embodiments, the at least one first and second electrodes may each be coupled to one of the at least one stage projection during an injection molding operation. In some embodiments, the main body may be injection molded. In some embodiments, the first face of the central region may include at least one rocker member. In some embodiments, the at least one sharp bearing body may be constructed of etched silicon. In some embodiments, the first face of the central region may be at least partially covered with an adhesive bearing member. In some embodiments, the device may further comprise an adhesive bearing member. In some embodiments, each of the at least one first electrode and each of the at least one second electrode may be micropenetrators. In some embodiments, each of the at least one first electrode and each of the at least one second electrode may be included on micropenetrators which are at least partially covered in an insulative material. In some embodiments, a first of the at least one second electrode may be configured to penetrate a first depth into a biological barrier and a second of the at least one second electrode may be configured to penetrate a second depth into the biological barrier. In some embodiments, first depth may be selected to be a shallow depth the second depth may be selected to be a subcutaneous depth. In some embodiments, the shallow depth may be an intradermal depth. In some embodiments, the device may further comprise at least one transmitter. In some embodiments, the at least one transmitter may be disposed on a second face of the central region opposite the first. In some embodiments, the analyte sensor device may be a glucose sensor.

In accordance with another example embodiment of the present disclosure an access assembly device for a biological barrier may comprise a main body including a central region and a peripheral region. The peripheral region may have a plurality of petal members. The device may further comprise a coupling on a first face of the central region. The device may further comprise at least one access member on an opposing face of the central region. The at least one access member may have a delivery lumen. The device may further comprise at least one analyte sensor having electrodes on the opposing face of the central region.

In some embodiments, the coupling may be a fitting such as a luer lock fitting. In some embodiments, the coupling may include at least one guide and at least one connector receiver. Each of the at least one connector receiver may have a ramped face and a ledge face. In some embodiments, the device further may comprise a septum sealing a passage in fluid communication with the at least one access member. In some embodiments, the device may further comprise at least one transmitter. In some embodiments, the opposing face of the central region may include at least one stage projection. The access members and the electrodes each may be coupled to one of the at least one stage projection. In some embodiments, the opposing face of the central region may include at least one rocker member. In some embodiments, the opposing face of the central region may include at least one stage projection. The at least one access member and the electrodes each may be coupled to one of the at least one stage projection and one of the at least one stage projections may form one of the at least one rocker member. In some embodiments, the analyte sensor may be a glucose sensor. In some embodiments, the at least one access member may include an array of microneedles extending from a sharp bearing body. In some embodiments, the electrodes may include micropenetrators extending from sharp bearing bodies. In some embodiments, the at least one access member may be constructed of etched silicon. In some embodiments, the at least one access member may include at least one first access member configured for shallow delivery and at least one second access member configured for subcutaneous delivery. In some embodiments, the at least one first access member may be configured for intradermal delivery. In some embodiments, the electrodes comprise a first set of electrodes configured to sense analyte levels at a shallow location in the biological barrier and a second set of electrodes configured to sense analyte levels at a subcutaneous location in the biological barrier. In some embodiments, the shallow location may be an intradermal location.

In accordance with still another example embodiment of the present disclosure an example access assembly device for a biological barrier may comprise a main body including a central region and a peripheral region. The peripheral region may have a plurality of petal members extending outwardly from the central region. The device may further comprise at least one coupling on a first face of the central region. The device may further comprise at least one first sharp bearing body on an opposing face of the central region. Each of the at least one first sharp bearing body may be in fluid communication with a respective one of the at least one coupling. The device may further comprise at least one second sharp bearing body on the opposing face of the central region. Each of the at least one second sharp bearing body may include at least one electrode. The device may include at least one first electrode and at least one second electrode which is associated with analyte sensing chemistry.

In certain embodiments of any of the devices described above, the main body may be configured to transition from a storage state to a deployed state. At least two portions of the main body may spreadingly displace as the main body transitions from the storage state to the deployed state. In certain embodiments of any of the devices described above, the main body may be configured to transition from a storage state to a deployed state. At least two adhesive bearing portions of the main body may spreadingly displace as the main body transitions from the storage state to the deployed state. Om certain embodiments of any of the devices described above, the main body may be configured to transition from a storage state to a deployed state. The central region may be configured to translate toward a biological barrier to which the device has been applied as the main body transitions from the storage state to the deployed state.

In accordance with another example embodiment of the present disclosure a medical agent administration system may comprise an infusion device. The infusion device may include a delivery assembly including at least one sensor and at least one pumping arrangement. The system may further comprise a set in fluid communication with the delivery device. The set may include at least one intradermal access member. The system may further comprise a controller configured to govern operation of the at least one pumping arrangement. The controller may be in data communication with the at least one sensor and configured to analyze data from the at least one sensor. The controller may be configured to determine a change in depth of the at least one access member has occurred based on the data from the at least one sensor.

In some embodiments, the system may further comprise at least one analyte monitor. In some embodiments, the system may further comprise a glucose monitor. In some embodiments, the controller may be in communication with at least one smart device and the infusion device. In some embodiments, the delivery assembly may be split between a first portion of the infusion device and a second portion of the infusion device removably coupled to the first portion. In some embodiments, the second portion may be a cassette assembly. In some embodiments, the cassette assembly may include at least one flow path and at least one valve component which are overlaid by at least one membrane. In some embodiments, the at least one sensor may include a pressure sensor configured to monitor volumes of fluid delivered from the infusion device to the set. In some embodiments, the controller may be configured to analyze the data from the at least one sensor to determine if a pressure decay rate is in breach of a predefined criteria. In some embodiments, the controller may be configured to analyze the data from the at least one sensor to determine if a pressure decay rate is in excess of a predefined threshold and configured to generate an alert for display on a user interface of the system when the pressure decay rate exceeds the threshold. In some embodiments, the controller may be configured to analyze the data from the at least one sensor to determine if a pressure decay rate is in below a predefined threshold and configured to generate an alert for display on a user interface of the system when the pressure decay rate is below the threshold. In some embodiments, the at least one sensor may include an acoustic volume sensor comprising a variable volume chamber and the data from the at least one sensor may be indicative of a volume of fluid in the variable volume chamber. In some embodiments, the controller may be configured to analyze the data from the at least one sensor to determine if change in volume of the variable volume chamber over time is in breach of predefined criteria. In some embodiments, the controller may be configured to analyze the data from the at least one sensor to determine if rate of change in volume of the variable volume chamber is in excess of a predefined threshold and configured to generate an alert for display on a user interface of the system when the rate exceeds the threshold. In some embodiments, the controller may be configured to analyze the data from the at least one sensor to determine if rate of change in volume of the variable volume chamber is below a predefined threshold and configured to generate an alert for display on a user interface of the system when the rate is below the threshold. In some embodiments, the at least one intradermal access member may include a microneedle. In some embodiments, the at least one intradermal access member may include an array of microneedles on a sharp bearing body coupled to a stage projection on a face of the set. In some embodiments, the face of the set may include at least one rocker member. In some embodiments, the set may include a main body having a central region and a peripheral region including a plurality of petal members. In some embodiments, the main body may be configured to transition from a storage state to a deployed state. At least two adhesive bearing portions of the main body may spreadingly displace as the main body transitions from the storage state to the deployed state. The central region may be configured to translate toward a biological barrier to which the set is applied as the main body transitions from the storage state to the deployed state. In some embodiments, the at least one sensor may be configured to generate a data signal which varies in relation to a delivery impedance from the at least one access member.

In accordance with another example embodiment of the present disclosure an example medical agent administration system may comprise an infusion device. The infusion device may include a delivery assembly including at least one sensor and at least one pumping arrangement. The system may further comprise a set in fluid communication with the delivery device. The set may include at least one shallow access member and at least one subcutaneous access member. The system may further comprise a controller configured to govern operation of the at least one pumping arrangement. The controller may be in data communication with the at least one sensor and configured to compare data from the at least one sensor related to fluid deliveries to the shallow access member and the at least one subcutaneous access member and determine a change in depth of one or more of the shallow access member and at least one subcutaneous access member has occurred based on the data from the at least one sensor.

In some embodiments, the at least one shallow access member and at least one subcutaneous access member may be microneedles. In some embodiments, the at least one shallow access member and at least one subcutaneous access member may each include an array of microneedles on a sharp bearing body coupled to a stage projection on a face of the set. In some embodiments, the face of the set may include at least one rocker member. In some embodiments, the set may include a main body having a central region and a peripheral region including a plurality of petal members. In some embodiments, the main body may be configured to transition from a storage state to a deployed state. At least two adhesive bearing portions of the main body may spreadingly displace as the main body transitions from the storage state to the deployed state. The central region may be configured to translate toward a biological barrier to which the set is applied as the main body transitions from the storage state to the deployed state. In some embodiments, the system may further comprise at least one analyte monitor. In some embodiments, the system may further comprise a glucose monitor. In some embodiments, the controller may be in communication with at least one smart device and the infusion device. In some embodiments, the delivery assembly may be split between a first portion of the infusion device and a second portion of the infusion device removably coupled to the first portion. In some embodiments, the second portion may be a cassette assembly. In some embodiments, the cassette assembly may include at least one flow path and at least one valve component which are overlaid by at least one membrane. In some embodiments, the at least one sensor may be configured to generate a data signal which varies in relation to a delivery impedance from the at least one access member. In some embodiments, the controller may be configured to determine the at least one subcutaneous access member has displaced to an intradermal depth when the delivery impedance related to the at least one subcutaneous access member indicated by the at least one data increases. In some embodiments, the controller may be configured to determine the at least one subcutaneous access member has displaced to an intradermal depth when the delivery impedance related to the at least one subcutaneous access member indicated by the at least one sensor increases to within a range of historical data related to the at least one intradermal access member. In some embodiments, the controller may be configured to generate an alarm when the delivery impedance related to the at least one subcutaneous access member indicated by the at least one sensor increases to within a range of historical data related to the at least one intradermal access member and the delivery impedance related to the at least one intradermal access member indicated by the at least one sensor decreases.

In accordance with another example embodiment of the present disclosure, a medical agent administration system may comprise an infusion device with a delivery assembly. The delivery assembly may include at least one pumping arrangement. The system may further comprise a set in fluid communication with the delivery device. The set may include at least one access member, a shallow analyte sensor, and a deep analyte sensor. The system may further comprise a controller configured to govern operation of the at least one pumping arrangement to selectively deliver fluid from the infusion device to the at least one access member. The controller may be in data communication with the shallow and deep analyte sensors. The controller may be configured to compare data from the shallow and deep analyte sensors and generate a notification when a relationship between data from the shallow and deep analyte sensors breaches a predefined criteria.

In some embodiments, the at least one shallow analyte sensor and at least one subcutaneous analyte sensor may include micropenetrators. In some embodiments, a face of the set may include at least one stage projection and the shallow and deep analyte sensors may be each coupled to one of the at least one stage projection. In some embodiments, the face of the set may include at least one rocker member. In some embodiments, the set may include a main body having a central region and a peripheral region including a plurality of petal members. In some embodiments, the main body may be configured to transition from a storage state to a deployed state. At least two adhesive bearing portions of the main body may spreadingly displace as the main body transitions from the storage state to the deployed state. The central region may be configured to translate toward a biological barrier to which the set is applied as the main body transitions from the storage state to the deployed state. In some embodiments, the shallow analyte sensor may be an intradermal analyte sensor and the deep analyte sensor may be a subcutaneous analyte sensor. In some embodiments, the shallow analyte sensor and deep analyte sensor may be glucose sensors. In some embodiments, the controller may be in communication with at least one smart device and the infusion device. In some embodiments, the delivery assembly may be split between a first portion of the infusion device and a second portion of the infusion device which may be removably coupled to the first portion. In some embodiments, the second portion may be a cassette assembly. In some embodiments, the cassette assembly may include at least one flow path and at least one valve component which are overlaid by at least one membrane. In some embodiments, the predefined criteria may be a time lag between changes in analyte level measured by the shallow analyte sensor and deep analyte sensor. In some embodiments, the predefined criteria may be determined from historical data collected from the shallow analyte sensor and deep analyte sensor. In some embodiments, the controller may be further configured to analyze data from the shallow and deep analyte sensors and generate a notification when data from either of the analyte sensors indicates the respective analyte sensor has displaced from its intended position.

In accordance with another example embodiment of the present disclosure, a medical agent administration system may comprise an infusion device with a delivery assembly. The delivery assembly may include at least one pumping arrangement. The system may further comprise a set in fluid communication with the delivery device. The set may include at least one shallow access member, at least one deep access member, and at least one an analyte monitor. The system may further comprise a controller configured to govern operation of the at least one pumping arrangement to selectively deliver fluid from the infusion device to each of the access members. The system may further comprise at least one sensor configured to generate a signal which varies in relation to a delivery impedance from the access members. The controller may be configured to analyze data from the sensor and the at least one analyte monitor and generate a notification when data from the at least one analyte monitor and data from the sensor respectively indicate at least one of the at least one analyte sensor and at least one of the access members has displaced from their intended positions.

In some embodiments, the at least one shallow access member and at least one deep access member may be microneedles. In some embodiments, the at least one shallow access member and at least one deep access member may each include an array of microneedles on a sharp bearing body coupled to a stage projection on a face of the set. In some embodiments, the face of the set may include at least one rocker member. In some embodiments, the set may include a main body having a central region and a peripheral region including a plurality of petal members. In some embodiments, the main body may be configured to transition from a storage state to a deployed state. At least two adhesive bearing portions of the main body may spreadingly displace as the main body transitions from the storage state to the deployed state. The central region may be configured to translate toward a biological barrier to which the set is applied as the main body transitions from the storage state to the deployed state. In some embodiments, the at least one shallow access member may be an intradermal access member and the at least one deep access member may be a subcutaneous access member. In some embodiments, the at least one analyte monitor may include a micropenetrator. In some embodiments, the controller may be in communication with at least one smart device and the infusion device. In some embodiments, the delivery assembly may be split between a first portion of the infusion device and a second portion of the infusion device removably coupled to the first portion. In some embodiments, the second portion may be a cassette assembly. In some embodiments, the cassette assembly may include at least one flow path and at least one valve component which are overlaid by at least one membrane. In some embodiments, the set may include a coupling configured to couple with a fluid transfer connector at a terminal end of a fluid line extending from the infusion device. In some embodiments, the set may include conductive traces extending from the at least one analyte monitor. The fluid transfer connector may include contacts configured to seat against the conductive traces when the fluid transfer connector is engaged with the coupling. In some embodiments, an electrical communication path may extend from the contacts and along the length of the fluid line.

In accordance with another example embodiment of the present disclosure an analyte sensing system may comprise an analyte sensor assembly including a shallow analyte sensor and a deep analyte sensor. The system may further comprise a controller in data communication with the shallow analyte sensor and the deep analyte sensor. The controller may be configured to compare data received from the shallow analyte sensor and the deep analyte sensor and generate a notification in the event that data from the analyte sensors deviate from an expected relationship by more than a threshold.

In some embodiments, the controller may be configured to initialize the expected relationship to a predefined anticipated relationship. In some embodiments, the controller may be configured to adjust the expected relationship based on data received from the shallow analyte sensor and the deep analyte sensor. In some embodiments, the controller is configured to set the expected relationship based at least in part on historical data received from the shallow analyte sensor and the deep analyte sensor. In some embodiments, the shallow analyte sensor may be an intradermal analyte sensor and the deep analyte sensor may be a subcutaneous analyte sensor. In some embodiments, the shallow analyte sensor and the deep analyte sensor may each include at least one micropenetrator. In some embodiments, the expected relationship may define a delay between changes in analyte levels sensed by the shallow analyte sensor and those sensed by the deep analyte sensor. In some embodiments, the analyte sensing assembly may include a coupling for a connector in hardwired communication with the controller. In some embodiments, the analyte sensing assembly may be configured to couple to a transmitter for wirelessly communicating data to the controller. In some embodiments, the analyte sensing assembly may include a face with at least one stage projection. The shallow and deep analyte sensors may each be disposed on the at least one stage projection. In some embodiments, the face may include a rocker member. In some embodiments, the analyte sensing assembly may include a main body having a central region and a peripheral region including a plurality of petal members. In some embodiments, the main body may be configured to transition from a storage state to a deployed state. At least two adhesive bearing portions of the main body may spreadingly displace as the main body transitions from the storage state to the deployed state. The central region may be configured to translate toward a biological barrier to which the analyte sensing assembly is applied as the main body transitions from the storage state to the deployed state.

### BRIEF DESCRIPTION OF THE DRAWINGS

These and other aspects will become more apparent from the following detailed description of the various embodiments of the present disclosure with reference to the drawings wherein:

FIG. 1 depicts a block diagram of an example system including a set, analyte sensor, and transmitter among other components;

FIG. 2 depicts a block diagram of an example system including a plurality of sets, an analyte sensor, and a transmitter among other components;

FIG. 3 depicts a block diagram of an example system including, among other components, an access assembly including an analyte sensor, transmitter, and at least one access member;

FIGS. 4A-4B depict an exemplary embodiment of a set;

FIG. 5 depicts a perspective view of an example embodiment of an access member;

FIGS. 6A-6B depict views of an example embodiment of an access member;

FIGS. 7A-7B depict views of an example embodiment of an access member;

FIG. 8A depicts a perspective view of an example embodiment of a number of access members on an example sharp bearing body;

FIG. 8B depicts a perspective view of an example embodiment of a number of access members on an example sharp bearing body;

FIG. 9A depicts a perspective view of an example embodiment of a number of access members on an example sharp bearing body;

FIG. 9B depicts a top plan view of an example embodiment of a number of access members on an example sharp bearing body;

FIG. 10A depicts a top plan view of an example embodiment of a number of access members on an example sharp bearing body;

FIG. 10B depicts a perspective view of an example embodiment of a number of access members on an example sharp bearing body;

FIG. 11A depicts a top plan view of an example embodiment of a number of access members on an example sharp bearing body;

FIG. 11B depicts a perspective view of an example embodiment of a number of access members on an example sharp bearing body;

FIG. 11C depicts a cross-sectional view taken at the indicated cut plane of FIG. 11A;

FIGS. 12A-12D depict various views of an exemplary access member;

FIG. 13 depicts a perspective view of an example set;

FIG. 14 depicts a top plan view of an example set;

FIG. 15 depicts a cross-sectional view taken at the indicated cut plane of FIG. 14;

FIG. 16 depicts a bottom plan view of an example main body of an example set;

FIG. 17 depicts a conceptual illustration of an example main body of an example set in a delivery state;

FIG. 18 depicts a view of an example set in place at an infusion site on a biological barrier;

FIG. 19 depicts an illustration of an example set in a delivery state at an infusion site on a biological barrier;

FIG. 20 depicts a side view of an example set;

FIG. 21 depicts a perspective view of an example set;

FIGS. 22A-22I depict example top plan views of a number of main bodies;

FIG. 23 depicts a perspective view of an exemplary set;

FIG. 24 depicts a perspective view of an example set with an example body partially exploded out of the set;

FIG. 25 depicts an exploded view of an example set in which an adhesive liner has been exploded off an adhesive member included on the set;

FIG. 26 depicts a perspective view of an example set;

FIG. 27 depicts a cross-section view taken along a medial plane of an example set;

FIGS. 28A-28B depict representational views of an example set respectively in a storage state and a delivery state;

FIGS. 29-30 depict conceptual views of an example set in a delivery state;

FIG. 31 depicts a perspective view of an example body having an example stage projection to which a number of example access members are coupled;

FIG. 32 depicts a perspective view of an example body;

FIG. 33 depicts a cross-sectional view of an example body;

FIG. 34 depicts a cut-away cross-sectional view of an example stage projection and example access member on an example sharp bearing body;

FIG. 35-36 depict views of an example body including a plurality of example stage projections each associated with at least one access member and each being fluidically isolated from one another;

FIG. 37-38 depict representational views of exemplary sets including rocker members;

FIG. 39A-B depict views of an exemplary main body;

FIG. 40 depicts a detail view of a portion of an example main body;

FIG. 41 depicts a top plan view of an example set;

FIG. 42 depicts a detailed view of the indicated region of FIG. 41;

FIG. 43 depicts a perspective view of an exemplary connector;

FIG. 44 depicts a perspective view of an example set;

FIG. 45 depicts a cross-sectional view of an example set;

FIG. 46 depicts a detailed view of the indicated region of FIG. 45;

FIG. 47 depicts an exploded view of an example analyte sensor and transmitter;

FIG. 48 depicts a bottom plan view of an example analyte sensor;

FIG. 49 depicts a cut-away perspective view of an example set of electrodes of an analyte sensor on an example stage projection;

FIG. 50 depicts a bottom plan view of an example access assembly;

FIG. 51 depicts a top plan view of an example access assembly and example connector;

FIG. 52 depicts a bottom plan view of another example access assembly;

FIG. 53 depicts a detailed view of the indicated region of FIG. 52;

FIG. 54 depicts a detailed view of the indicated region of FIG. 52;

FIGS. 54-57 depict a variety of exemplary adhesive member on example sets; and

FIGS. 58-63 depict block diagram view of example delivery assemblies which may be included in certain example infusion devices.

### DETAILED DESCRIPTION

In various embodiments, a set may be used in conjunction with an infusion device, system, and related method. In various embodiments, example sets may be configured to be inserted into a layer of a user's skin and be fluidly connected to a fluid source. In various embodiments, example sets may be fluidly connected to a length of tubing and/or to an infusion device. Infusion devices include any infusion pump and may include, but are not limited to, the various infusion devices described in U.S. patent application Ser. No. 13/788,260, filed Mar. 7, 2013 and entitled Infusion Pump Assembly, now U.S. Publication No. US-2014-0107579, published Apr. 17, 2014 (Attorney Docket No. K40); U.S. Pat. No. 8,491,570, issued Jul. 23, 2013 and entitled Infusion Pump Assembly (Attorney Docket No. G75); U.S. Pat. No. 8,414,522, issued Apr. 9, 2013 and entitled Fluid Delivery Systems and Methods (Attorney Docket No. E70); U.S. Pat. No. 8,262,616, issued Sep. 11, 2012 and entitled Infusion Pump Assembly (Attorney Docket No. F51); and U.S. Pat. No. 7,306,578, issued Dec. 11, 2007 and entitled Loading Mechanism for Infusion Pump (Attorney Docket No. C54); all of which are hereby incorporated herein by reference in their entireties. Any connectors which couple a fluid line from a pump to an infusion set described in the above referenced applications may be used with the sets described herein or any of the connectors described in U.S. patent application Ser. No. 16/797,624, filed Feb. 21, 2020 and entitled Infusion Set and Inserter Assembly Systems and Methods, now U.S. Publication No. US-2020-0289748, published Sep. 17, 2020 (Attorney Docket No. 00101.00307.AA159) also incorporated by reference herein in its entirety. Microneedles described herein may include, but are not limited to, the various microneedles described in U.S. Pat. No. 11,154,698, issued Oct. 26, 2021, and entitled Microneedle Systems and Apparatus (Attorney Docket No. G34) or U.S. Pat. No. 5,983,136, issued Nov. 9, 1999, and entitled System for Delivery of Drugs by Transport (Attorney Docket No. B60).

Various embodiments are described and shown herein. Each embodiment of each element of each device may be used in any other device embodiment.

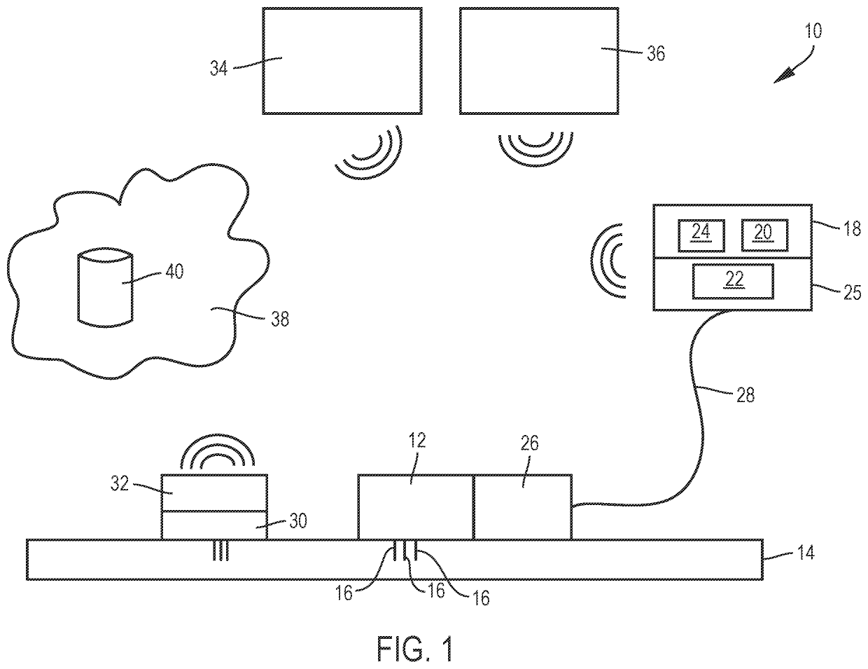

Referring now to FIG. 1, a block diagram of an exemplary system 10 is depicted. The exemplary system 10 may include a set 12. The set 12 may be any of the sets 12 described herein. The set 12 may adhere to a patch of skin 14 of a patient and establish access with a delivery destination in the patient. The set 12 may include at least one access member 16 which may allow for fluid flow from the set 12 out of each access member 16 and into the delivery destination. Each of the access members 16 may be an indwelling body which extends at least partially into the skin during use of the set 12. The delivery destination may be a subcutaneous destination, though in other embodiments may be a shallow delivery destination. Where the delivery destination is a shallow delivery destination, the at least one access member 16 may deliver fluid into a portion of the skin between the stratum corneum and subcutaneous tissue. Shallow delivery destinations may include an epidermal or dermal target location or may, for example, target a junctional area between the epidermis and dermis or dermis and subcutis. The delivery destination may be an intradermal delivery destination. The at least one access member 16 may be any of the access members 16 described herein and may include at least one delivery sharp such a microneedle in certain examples. In certain examples, a set 12 may include access members 16 which have different characteristics. The system 10 may delivery independently to one or more of the access members 16 or any delivery may be communicated to all of the access members 16. Any combination of access members 16 described herein may be used. There may be one or more first access member 16, one or more second access member 16, one or more third access member 16 and so on included in a single set 12. Such access members 16 may, for example, penetrate different depths into a patient. In some examples, the set 12 may include at least one subcutaneous access member 16 and at least one shallow destination access member 16. Alternatively, a set 12 may include access members 16 which extend to different shallow delivery destinations when the set is in place on a patient.

For delivery of certain agents, it may be desirable that the set 12 include at least one access member 16 appropriate for shallow delivery. Due to the high degree of vascularization of dermal region, for example, absorption of agent may occur more rapidly than if the same agent where to be administered into a subcutaneous destination. This may, for example, allow an injection of an agent such as insulin to be faster acting. Thus, such shallow delivery may facilitate tighter control of analyte levels of interest. Shallow delivery may also result in less variability in rates of absorption between patients or between different infusion sites on the same patient. Additionally, certain agents such as insulin may come in different varieties. For example, insulin types may include rapid, short, intermediate, and long acting insulin which differ, e.g., in how quickly and how long they will act to reduce blood glucose levels. Due to the faster absorption in the intradermal region, it may be possible to deliver types of agent with slower onset of action and observe onset times closer to comparable with the onset time of a more rapid onset agent administered subcutaneously. Shallow injection may also increase patient compliance and quality of life. This may be especially true for certain patient populations such as those with juvenile diabetes. Application of set 12 with access member(s) 16 for delivery into a shallow delivery destination may be painless as the access member(s) 16 may be too short to reach nerve endings which are located deeper in the anatomy. Additionally, certain types of access member(s) 16 may be better tolerated by patients. Silicon microneedles, for instance, may not have these same allergy concerns as access member(s) 16 formed from materials including nickel (e.g. stainless steel).

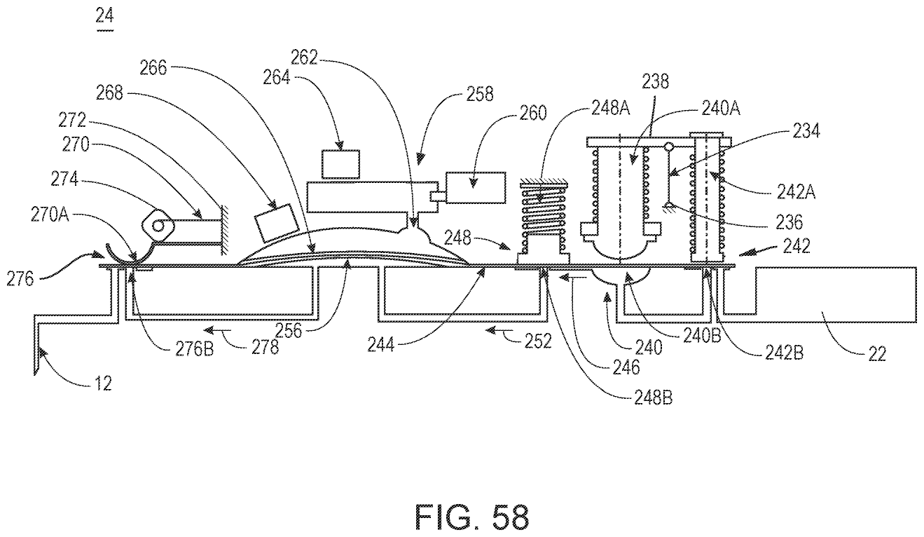

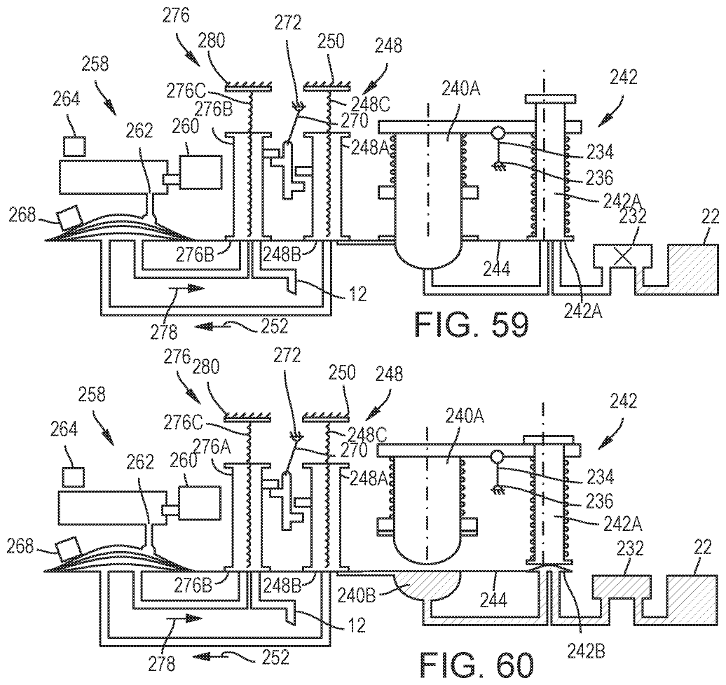

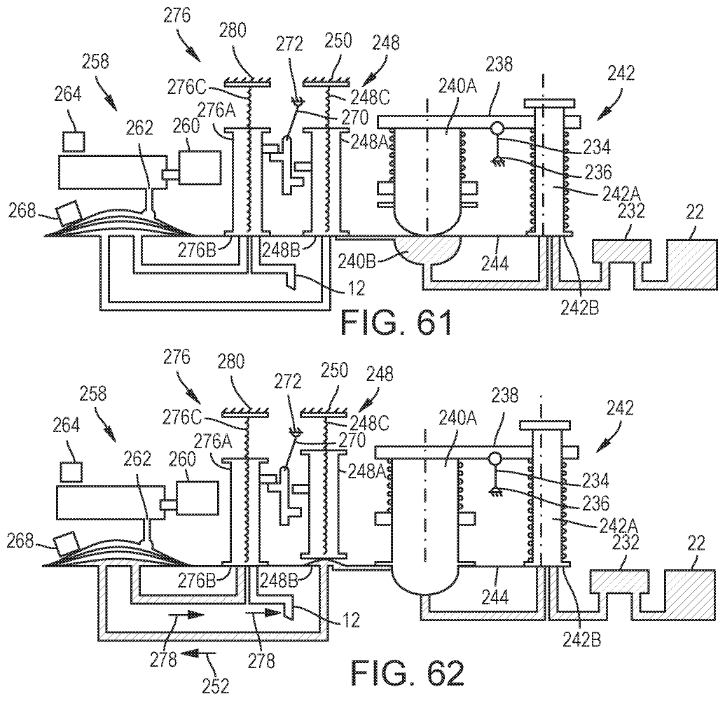

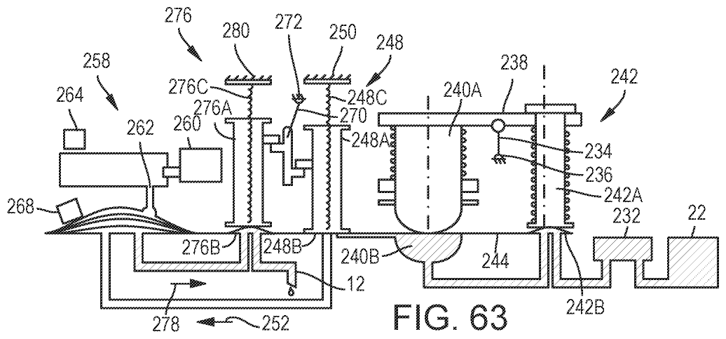

The set 12 may fluidically communicate with an infusion device 18. The infusion device 18 may include a controller 20 which may govern operation of a delivery assembly 24 (e.g. pumping components, valves, sensors monitoring pumping components or configured to provide data related to aspect of fluid delivery from the infusion device, etc.) to output desired volumes of fluid from a reservoir 22 associated with the infusion device 18. Multiple controllers 20 may be included in certain embodiments and at least one of the controller 20 may be disposed outside of the infusion device 18. An example delivery assembly 24 is depicted and described in relation to FIGS. 58-63. In certain embodiments, the infusion device 18 may include a reusable component and a disposable component which may be removably coupled to on another. In the example shown in FIG. 1, the infusion device 18 includes a cassette assembly 25 which may attach to a reusable portion of the infusion device 18. The cassette assembly 25 may include a reservoir 22 and may be replaced when fluid in the reservoir 22 has been depleted. The delivery assembly 24 may be split between the cassette assembly 25 and the reusable portion. The cassette assembly 25 may, for example, include fluid path ways and valves which may be acted on through a flexible membrane overlaying at least a portion of the cassette 25. The reusable portion may include any of the controller 20, a power source (e.g. battery), a speaker, a user interface, wireless communication hardware, and various sensors and actuators to govern dispensing of fluid through the cassette assembly 25.

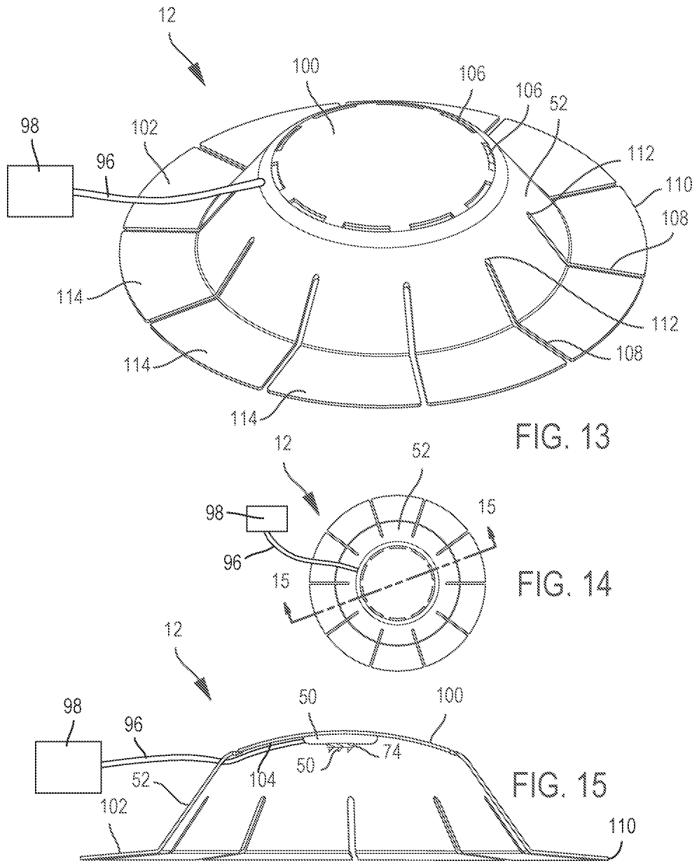

The infusion device 18 (e.g. an outlet of cassette assembly 25) may be fluidically coupled to the set 12 via a connector 26 (see, e.g., FIG. 43). In alternative embodiments, an outlet of a cassette assembly 25 could be hard plumbed to the set 12. In the example embodiment, the connector 26 is tethered to the infusion device 18 via run of tubing 28, however, in alternative embodiments, the connectors 26 described herein may be provided as part of the infusion device 18 (e.g. cassette assembly 25) and the intermediary tubing 28 may be omitted. Any suitable connector 26 may be used and a set 12 may include a cooperating interface which may engage in fluid tight manner with the connector 26. In certain embodiments, the connector 26 may be a luer lock fitting which may engage with a luer fitting included as part of the set 12. In other embodiments, the connector 26 may be any of the connectors described in any of the documents incorporated by reference herein. In certain examples, the connector 26 may engage with a coupling 98 (see, e.g., FIG. 13) which is attached to the set 12 via a run of set tubing 96 (see, e.g., FIG. 13). Where a luer fitting is used, the luer fitting may be associated with a check valve to prevent fluid flow when disengaged with the set 12. Caps or covers may be included to cover the connector 26 and/or coupling when in a disconnected state.

The infusion device 18 may deliver any desired fluid to the delivery destination via the set 12. In various examples, the infusion device 18 may deliver at least one medical agent. Agents supplied may include drugs which are generally supplied as a continuous or substantially continuous infusion though other drugs may also be used. This may include small molecules, biologicals, recombinantly produced pharmaceuticals, and analogs thereof. In various examples, the infusion device 18 may deliver an agent which affects the cardiovascular system or blood vessels. For example, an infusion device 18 may deliver a vasodilator. In certain examples, a drug for the treatment of pulmonary arterial hypertension such as Treprostinil may be delivered. In some examples, an infusion device 18 may deliver a peptide such as a regulatory hormone. In some examples, the agent may be a drug for the treatment of diabetes or a drug which acts to alter blood glucose levels. In certain examples, the infusion device 18 may deliver insulin. In certain embodiments, an infusion device 18 may deliver glucagon. Chemotherapy drugs may also be delivered via the set 12. In some embodiments, multiple agents may be delivered by one or more infusion devices 18 of the system 10. For example, any of the agents described above may be delivered. Any of the agents delivered may be delivered with one or more excipient which may or may not have help to increase absorption. Using insulin as an example, niacinamide may, for example, be delivered. Where references to insulin, glucagon, blood glucose, diabetes, etc. are described herein, their use is merely exemplary and it shall be understood, that use for other medical conditions or with other drugs or other analytes is contemplated.

In various embodiments, the system 10 may also include one or more analyte sensors 30. The analyte sensor(s) 30 may generate data related to a level of an analyte of interest in a patient. The analyte sensor(s) 30 may include an amperometric sensor which generates an electric current which is in proportion to the level of an analyte of interest in the patient. The analyte sensor(s) 30 may include one or more electrode arrangement and may be covered, coated, or otherwise associated with an enzyme specific the analyte of interest to facilitate such sensing. Any chemistry known for a desired analyte may be used. The enzyme may in some examples be an oxidoreductase from enzyme commission group EC 1.1. The enzyme may be categorized in enzyme group EC 1.1.3 in some embodiments. In certain examples, glucose oxidase may be used, though any suitable sensor chemistry for the analyte of interest may be used. Some examples may include an exclusion membrane which blocks, for example, large molecules, molecules above a certain molecular weight (e.g. if glucose is the analyte of interest), or interfering substances may separate the enzyme from the rest of the patient anatomy. Additionally, certain embodiments may include a membrane which may increase biocompatibility. In certain implementations, an analyte sensor 30 may be a blood glucose monitor. Other analytes may be monitored as well. The analyte sensor(s) 30 may monitor fluid in a subcutaneous space in some examples.

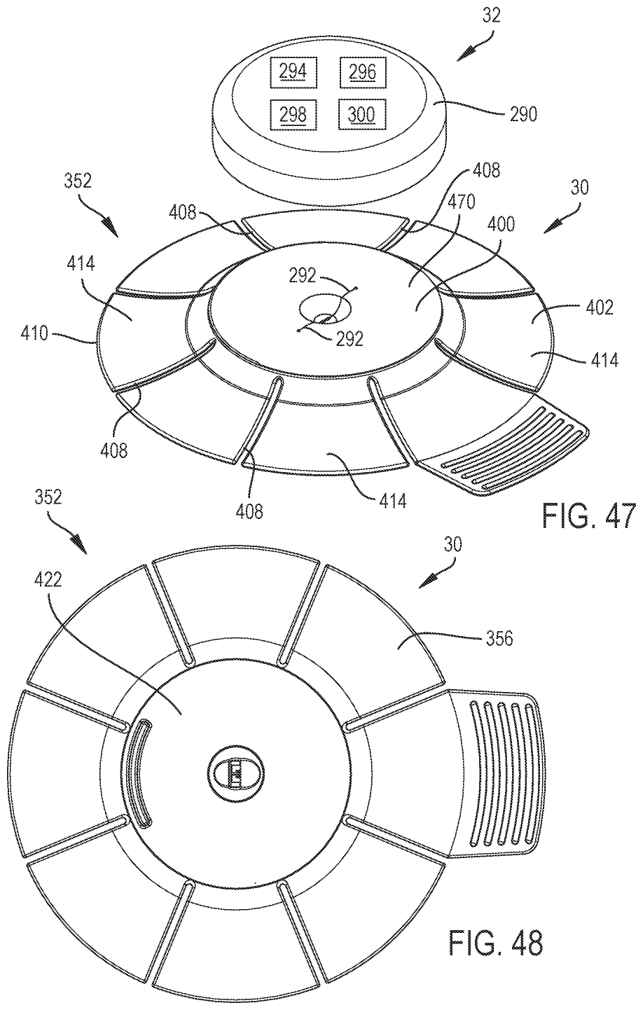

In alternative examples, the analyte sensor(s) 30 may monitor patient fluid at least at a shallow location. The analyte sensor(s) 30 may, for example, monitor fluid in a portion of the skin between the stratum corneum and subcutaneous tissue. Shallow sensing locations may include an epidermal or dermal target location or may, for example, target a junctional area between the epidermis and dermis or dermis and subcutis. The sensing location may be an intradermal location in some examples. The analyte sensor(s) 30 may be any of those described herein for example in relation to FIGS. 47-49.

The analyte sensor(s) 30 may include or be associated with a transmitter 32. The transmitter 32 may be included within a housing for an analyte sensor 30 or may alternatively be a separate component which may, for example, dock with the sensor 30 and transmit data collected by the analyte sensor 30. The transmitter 32 may be a wireless transmitter such as a radio frequency based transmitter 32. For example, the transmitter 32 may be a near field communication transmitter 32 or may be a Bluetooth transmitter 32. In some embodiments, multiple types of transmitters may be included in a transmitter 32 (e.g. NFC and Bluetooth). Analyte sensor(s) 30 and/or transmitters 32 may include a power source (e.g. coin cell battery) and may include a memory for storing sensor data. Analyte sensor(s) 30 may transmit sensor data via a transmitter 32 upon interrogation by another component of the system 10 or may transmit sensor data based upon a predefined schedule. For example, a transmitter may transmit sensor data after some preset number of minutes (e.g. 1-5 minutes) has elapsed since last transmission. Where transmission is based on a predefined schedule, data may also be transmitted upon interrogation by another component of the system 10. The data transmitted may be data from individual sensor readings or may be numerically processed data from an analyte sensor 30 (e.g. average of sensor readings over some period of time). Individual sensor readings may be taken at a given time or each individual sensor reading may be rendered from integrated sensor signal over some period of time. Any of the analyte sensors 30 described herein may forego a transmitter 32 and instead have (or establish when data transfer is desired) a wired or other physical connection to another component of the system 10. In embodiments where a transmitter 32 is included, wired connection may still be possible to access data from the sensor 30 if desired.

In certain examples, the transmitter 32 may also transmit alarms in the event that data from an analyte sensor 30 warrants alerting a user of the system 10. These alarms may be transmitted by the transmitter 32 independent of any predefined schedule and/or without the need for any interrogation by another component of the system 10. In some embodiments, sensor data may be transmitted by a first type of transmitter (e.g. NFC) included in the transmitter 32 and alarms may be transmitted by a second type of transmitter (e.g. Bluetooth) in the transmitter 32.

Sensor data may be transferred to the infusion device 18, a first interface 34 of the system 10, a second interface 36 of the system 10 (third, fourth, fifth, etc. interfaces may also be included in some examples), a database 40 in the cloud 38, or some combination thereof. Additionally, data may be transmitted to one or more of the above and that component may then pass the data to other components of the system 10. In certain examples, the first interface 34 may be a reader for the analyte sensor(s) 30. The reader may be a dedicated reader or may be a smart device such as a smart phone in some embodiments. The second interface 36 may be a smart device. In some examples, the first interface 34 may be a smart phone and the second interface may be a smart watch, tablet, or second smart phone (e.g. that of a parent, guardian, caregiver, etc.). Any of the infusion device 18, first or second interface 34, 36, or cloud 38 may generate and transmit an alarm to other components of the system 10 should data received meet certain predefined criteria.

The infusion device 18 may receive data from the transmitter 32 (directly or indirectly) and the controller 20 of the infusion device 18 may analyze this data to inform administration of agent from the infusion device 18. Delivery of agent may thus be closed loop in certain examples. Delivery of agent in other examples may be open loop, but data from the analyte sensor(s) 30 may, for example, be used to inform generation of alerts by the controller 20.

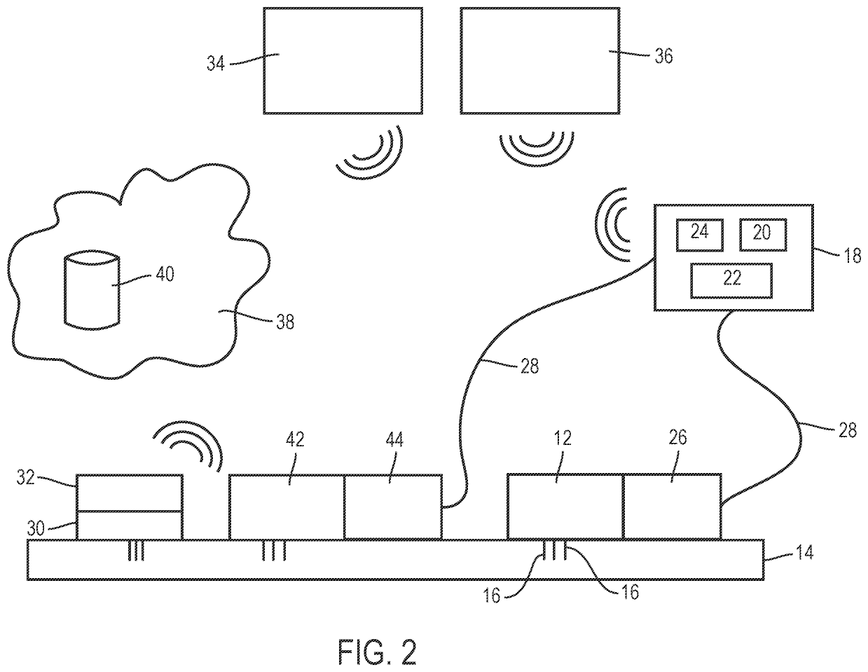

The controller 20 of the infusion device 18 may initiate or halt delivery of agent in the event that the analyte level or analyte level trend is determined to be in breach of a threshold or outside of a predefined range. The controller 20 may also adjust delivery of agent in the event that certain criteria are met. For example, the controller may increase or decrease an infusion rate in the event that data from the analyte sensor(s) 30 indicate analyte levels are changing in correspondence with a predefined trend. In some embodiments where the agent serves to decrease analyte levels, if analyte levels are decreasing, the delivery rate may be lowered or delivery may be halted. If analyte levels are increasing, the delivery rate may be increased by the controller 20. The opposite may be true in scenarios where the agent serves to increase the analyte level. In certain embodiments, the infusion device 18 may pump fluid to the set 12 at a basal rate and may occasionally provide a bolus to the patient. Depending on the rate of change in the analyte level, the basal delivery rate may be adjusted by the controller 20 or the controller 20 may orchestrate administration of a bolus (or the basal rate may be adjusted in addition to the administration of a bolus). Where multiple agents are delivered (e.g. to different sets 12, see, e.g. FIG. 2) by one or more infusion device 18, the type of agent delivered may be altered. For instance, if an agent which decreases analyte levels is being administered and analyte levels are decreasing at greater than a certain rate, delivery may switch to an agent which serves to increase analyte levels (and vice versa).

Referring now to FIG. 2, in some embodiments, the system 10 may include a first set 12 and a second set 42. Each of the sets 12, 42 may be in fluid communication (e.g. via tubing 28 or directly connected) to an infusion device 18. Alternatively, each set 12, 42 may be connected to a separate infusion device 18 (only one shown in FIG. 2). In the example shown, each of the sets 12, 42 may interface respectively with a first connector 26 and a second connector 44. Each of the sets 12, 42 may be identical to one another though could differ in certain examples. Similarly, each of the connectors 26, 44 may be identical though they could differ in alternative embodiments. In some embodiments, infusion device(s) 18 may supply a different agent to each of the sets 12, 42 and fluidically isolated reservoirs 22 containing the agents may be included (though in some instances the same agent may be supplied to both). Where a single infusion device 18 is used, the infusion device 18 may have separate reservoirs 22 for each agent and may include multiple delivery assemblies 24. In certain examples, the sets 12, 42 may have different delivery destinations. For example, one set 12, 42 may deliver to a shallow destination while the other may delivery to a subcutaneous destination. In embodiments where different agents are delivered to the patient via the sets 12, 42, the agents may be related in affect. For example, each of the agents may alter the same analyte. One of the agents may be a hormone, the other of the agents may also be a hormone which may counter the effect of the first hormone. Using the non-limiting example of a system 10 for use in treatment of diabetes, one of the agents may be insulin and the other of the agents may be glucagon. Further sets may be included in other examples. Additionally, any suitable number of agent reservoirs 22 in any suitable number of infusion devices 18 may be included.

In alternative embodiments, each of the sets 12, 42 may be combined into a single assembly with flow paths and access members 16 associated with each respective connector interface for the connectors 26, 44. Each set of flow paths and access members 16 may be isolated fluidically from other sets of flow paths and access members 16 within the single assembly. Thus one agent may be delivered via the first connector's 26 coupling to the set 12 and a second agent may be delivered via the second connector's 44 coupling to the set 12. The agents would remain out of fluid communication with one another in the delivery assembly.

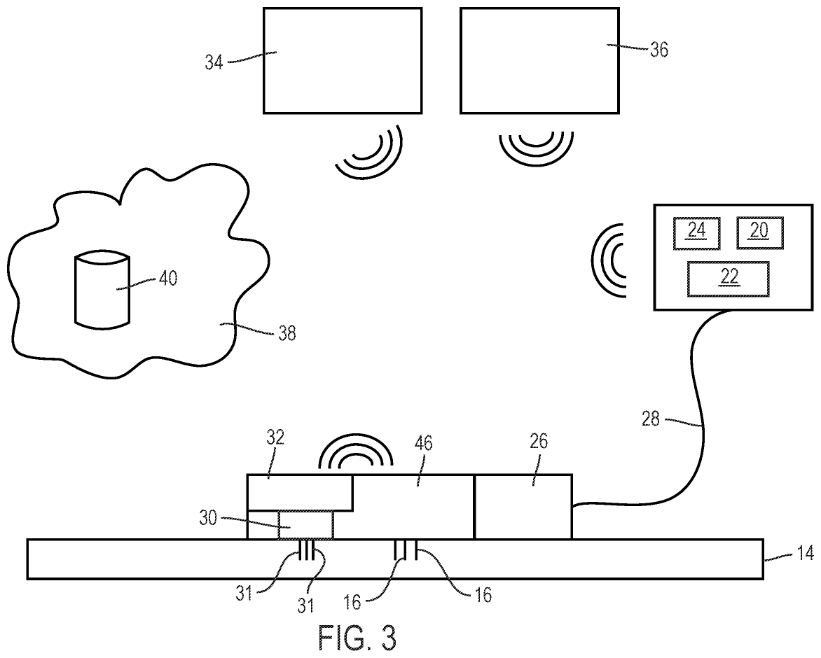

Referring now to FIG. 3, in certain examples, the system 10 may not include a separate set 14 and analyte sensor(s) 30. Certain examples may include an access assembly 46 which may include one or more access member 16 (e.g. microneedle array) for delivery of agent to a delivery destination as well as at least one analyte sensor 30. The access member(s) 16 and indwelling portion(s) 31 of the analyte sensor 30 may be spaced at least a certain distance from one another. In certain examples, they may be 5-15 mm apart (e.g. 7-11 or more millimeters apart).

The indwelling portion(s) 31 of the analyte sensor may be microneedles in certain examples. In some embodiments, the indwelling portion(s) 31 may be microneedles with no flow lumen 68 or bore. Such indwelling portions 31 may be referred to as micropenetrators. Micropenetrators may be constructed of etched silicon and may in some embodiments act as electrodes of the analyte sensor(s) 30. The silicon used may be doped to optimize conductivity for use of the micropenetrators as electrodes. In other embodiments, micropenetrators may be coated at least partially with an insulative material and one or more conductive electrode may be included on the surface of the insulative material. Known analyte sensor arrangements may also be incorporated into a set 14 in alternative embodiments.

The access assembly 46 may be in fluid communication with an infusion device 18 via tubing and a connector 26 (see, e.g., FIG. 43) which may engage with the access assembly 46. In some examples, multiple agents may be delivered via the access assembly 46 and multiple connectors 26, 44 may couple to the access assembly 46. In such examples, the fluid delivery portion of the access assembly 46 may be arranged as described above in relation to FIG. 2.

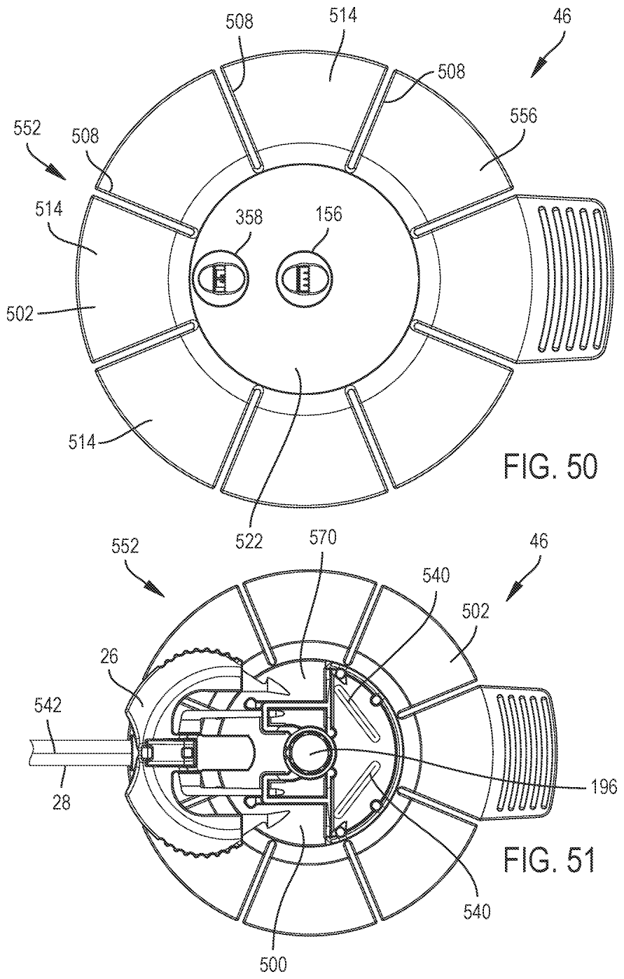

A transmitter 32 may also be included in the access assembly 46. Alternatively the transmitter 32 may dock with the access assembly 46 and transmit data received from any analyte sensor(s) 30. Systems 10 including an access assembly 46 may also include one or more individual analyte sensor(s) 30 or set 12, 42 which is/are distinct from the access assembly 46. In other embodiments, data from the sensor(s) 30 may be transferred via a wired connection. The connector 26, may for example, include a contacts and be associated with a wire leading to the infusion device 18. Example embodiments of access assemblies 46 are shown and described in relation to FIGS. 50-54.

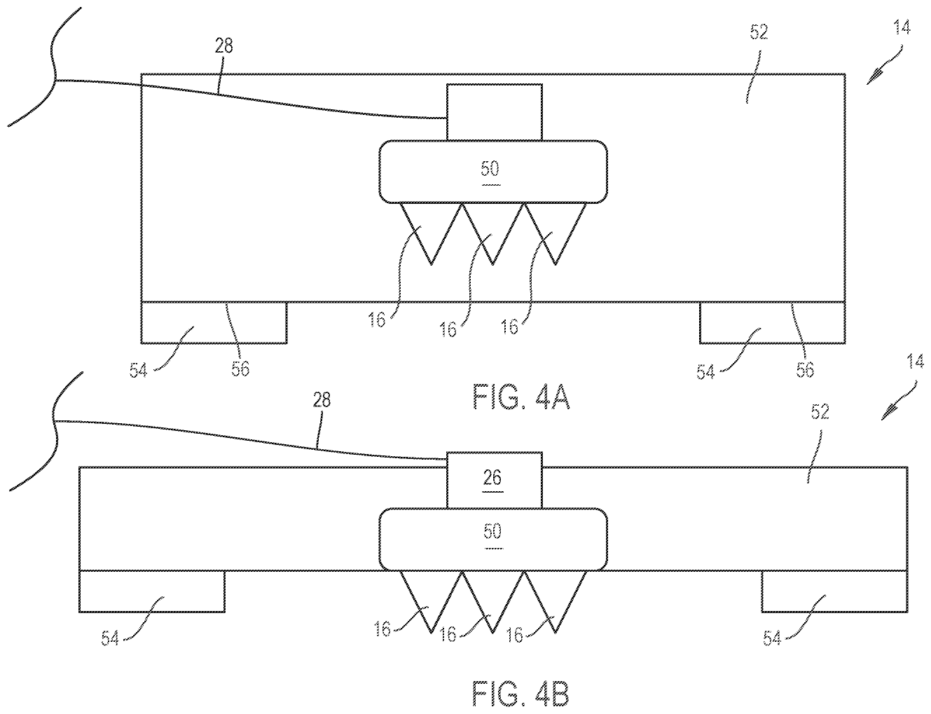

Referring now to FIG. 4A and FIG. 4B, an embodiment of an exemplary set 12 is depicted. The example set 12 may be a low profile set 12 which may be applied over the skin of a patient. The example set 12 may be sized to be easily applied to a wide variety of injection sites over a patient's body and may be easily concealable under clothing. Additionally, the example set 12 may be designed for simple manual application. Thus, an inserter assembly for placing the set 12 at an infusion site and facilitating introduction of any access member(s) 16 of the set 12 into the patient may not be needed.

Such sets 12 may be used to dispense an agent into a target delivery destination of a patient via one or more access member 16. In the example embodiment, a plurality of access members 16 are included in the set 12, though other embodiments may only include a single access member 16. The exemplary plurality of access members 16 may be arranged in a one or two dimensional array and extend from a body 50 to which a connector 26 communicating with an infusion device 18 may be coupled. Where multiple access members 16 are included, the access members 16 may be arranged in one or more rows and/or columns. Though three access members 16 arranged in a single row are depicted in FIG. 4A, the number and arrangement of access members 16 may differ in alternative embodiments. Any suitable number of rows and/or columns may be included in various examples. In various embodiments there may, for example, be a single row array of access members 16 including up to five access members 16. Preferably, the access members 16 may be arranged so as to prevent a bed of nails type scenario in which penetration of the skin via the access members 16 may be inhibited or inconsistent across users or sets 12. This may occur when too many access members 16 are arranged in close proximity to one another. Thus, the array may be referred to as a spaced array of access members 16. Each of the access members 16 may have the same height or at least one of the access members 16 may have a height which differs from at least one other access member 16 of the set 12. In the example embodiment shown in FIGS. 4A-4B, the access members 16 are depicted as delivery sharps. The delivery sharps shown in FIGS. 4A-4B are microneedles. Such microneedles may be present in delivery devices 10 with shallow (e.g. above subcutaneous tissue) target delivery destinations. Microneedles which extend slightly into subcutaneous tissue may be present in certain examples.

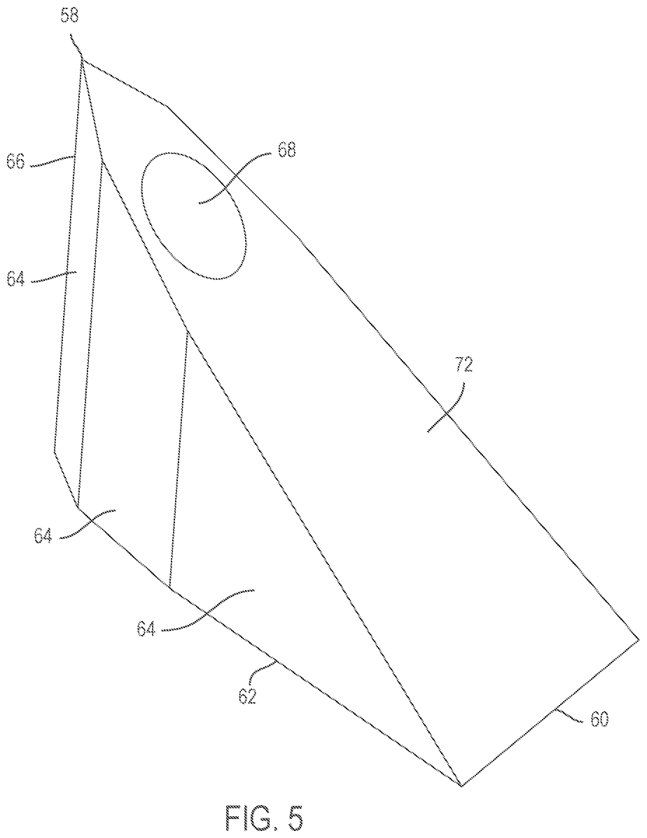

Referring now also to FIG. 5, where microneedles are used, the microneedles described herein may, in certain embodiments, be MEMS produced, polyhedral (e.g. pyramidal), silicon crystal microneedles. These microneedles may be no greater than 1 mm in height, e.g. 0.6 mm or 0.8 mm (though longer or shorter microneedles may also be used). In some embodiments, microneedles may be 1200-1500 microns in height or perhaps longer in some examples. In some embodiments, microneedles may have a height sufficient to puncture at least some distance into subcutaneous tissue. At least some edges of the microneedles may be rounded or filleted, though such microneedles may still be referred to herein as polyhedral. In some examples and as shown in FIG. 5, the microneedles described herein may be generally in the shape of a heptagonal prism (though pentagonal, nonagonal, and other polygonal prisms may also be used as the base shape) which has been diagonally sected to form a heptagonal ramp or pointed wedge. In such embodiments, the heptagonal prism may be sected by a plane extending from a vertex 58 of the top face of the prism through the most distal side 60 of the base 62. At least two sides of the base of the microneedle may be parallel. The side walls 64 may extend substantially perpendicularly from the base 62. A microneedle may be substantially symmetric about a line of symmetry extending from the vertex 58 to a point above the center of the most distal side 60. In other embodiments, a microneedle may be conically shaped. Any other suitable shape may be used. In the example, the vertex 58 is shown as a point which forms a tip of the microneedle. In other embodiments, this portion of a microneedle may be rounded (though may still be referred to herein as a vertex 58 and such microneedles may still be referred to as pointed). In such embodiments, the back facing edge 66 may be a round face or the back facing edge 66 and the adjacent side walls 64 may be replaced by a rounded face.

The points or tips of microneedles described herein may be solid and the flow lumens 68 through the microneedles may be offset from the points or tips (in FIG. 5 the vertex 58 forms the tip) of the microneedles. Hollow tipped microneedles in which the flow lumen 68 extends to the tip of the microneedle may also be utilized. In some embodiments, the microneedles may be NanoPass hollow microneedles available from NanoPass Technologies Ltd. of 3 Golda Meir, Nes Ziona, Israel. It should be noted that microneedles (or the substrate on which they are disposed) described herein as constructed of silicon may have a surface layer of silicon dioxide (which may, for example, form with exposure to air) while still being considered constructed of silicon.

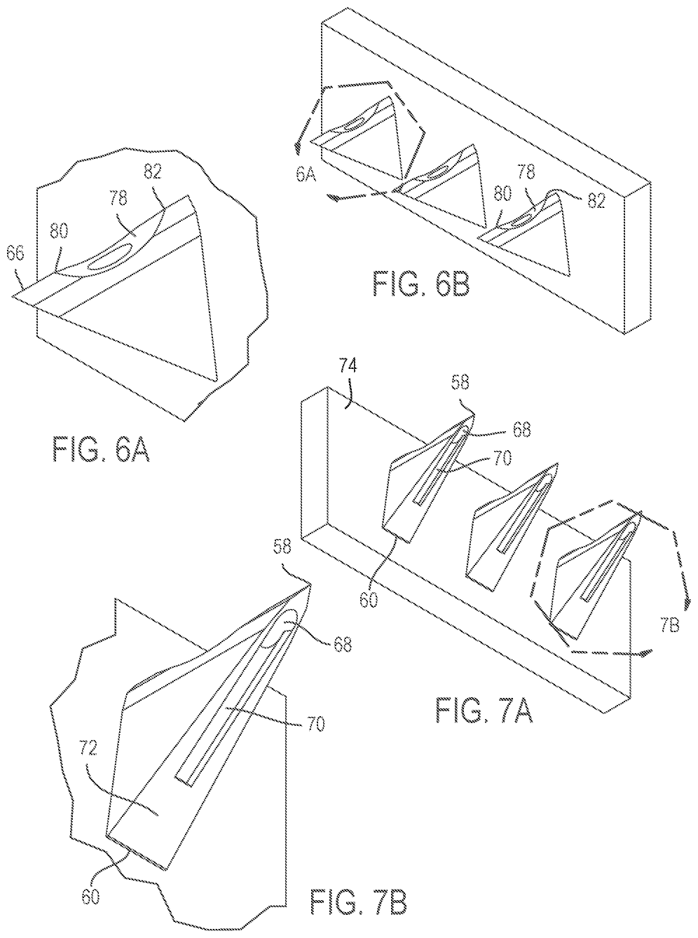

With reference to FIGS. 6A-7B, in some embodiments, microneedles may be constructed to include certain features that may help to reduce the pressure required to inject fluid, such as a medical agent, into the skin of a patient. In some examples, features common certain to insect stingers or biological venom administration structures may be incorporated. These features may include various recesses or depressions which are formed as part of each microneedle or at least one microneedle of a set 12. These recesses or depressions may fluidly communicate with the flow lumen 68 of the respective microneedle. In some embodiments, different microneedles of a set 12 may include different recesses or some microneedles may include a plurality of recesses which could be of different varieties (though need not be).

For example, as shown in FIGS. 6A-7B, a microneedle may include a channel or trough 70 on an exterior sloped face 72 leading from the flow lumen 68 toward the distal side 60. The channel 70 may allow medical agent to flow through it along the outer side of the microneedle to find a path of least resistance, or weakest link, into the skin. In the embodiments shown, medical agent may be routed by the channel 70 to flow along the outer side of the microneedle to a weak region in the skin in the event the outlet of the flow lumen 68 has been inserted to a greater depth than the depth of the weak region. The lamina lucida junction, an intradermal delivery destination, is a weak link in the skin structure, and is difficult to consistently inject directly into due to its relative thinness (it is typically on the order of 40 nm thick). A microneedle including a channel 70 may, for example, allow flow of medical agent to the lamina lucida junction when the lamina lucida junction has been passed by the outlet of the flow lumen 68. The channel 70 may facilitate distribution of the medical agent through a larger area of entry or injection. In some examples, incorporating a channel 70 into a microneedle may reduce the pressure required to inject a medical agent into the skin considerably. In certain examples pressure may be reduced by 600% or more (e.g. from 120 pounds per square inch (psi) to from 18 to 20 psi in certain examples).

An appropriate silicon etching technique (or mold in embodiments using polymeric microneedles) may be used to create steeper side walls of the channel 70. This may help inhibit the skin from bending into and occluding the channel 70. Etching techniques that could be used include, by way of non-limiting example, chemical etching techniques (e.g., acid). Suitable etching techniques may include ion based etching techniques (e.g. reactive ion etching). The etching process could be a wet etching process or a dry etching process. In some non-limiting embodiments, the channel 70 may be within a range of 50-60 microns wide from side to side. In some non-limiting embodiments, the flow lumen 68 may have a diameter of 50-60 microns. The channel 70 may have a width equal to the diameter or widest portion of the flow lumen 68 or the channel 70 may have a width which is less than or greater than the width of the flow lumen 68. In certain examples, the width of the channel 70 may be about 5-10 percent of the height of the microneedle.

To avoid leakage of the fluid from the channel 70, it may be desirable to ensure that the channel 70 terminates at least a certain distance beneath the surface of the skin yet also reaches the targeted skin layer (e.g., the lamina lucida junction) when the microneedle is inserted into the skin. In some embodiments the channel 70 extends from the flow lumen 68 to within at most 50 microns (e.g. 50-200 microns) of the base 62 of the microneedle. In some embodiments, the end of the channel 70 most proximal the base 62 of the microneedle may be at least below the stratum corneum (and perhaps one or more of the stratum lucidum, stratum granulosum, stratum spinosum, and stratum basale) when the microneedle is inserted into the skin. In some embodiments, the end of the channel 70 most proximal the base 62 may be disposed below the epidermis (e.g. in the basement membrane) or within the epidermis.

The channel 70 need not be straight or shaped in the manner shown in and described with reference to FIGS. 7A-7B. In some embodiments, the channel 70 may be a more meandering channel 70. A curved channel 70 could, for example, be used provided the dimensions of the microneedle are accommodated. Moreover, there need not be only one channel 70. More than one channel could be used provided structural integrity of the microneedle is accommodated.

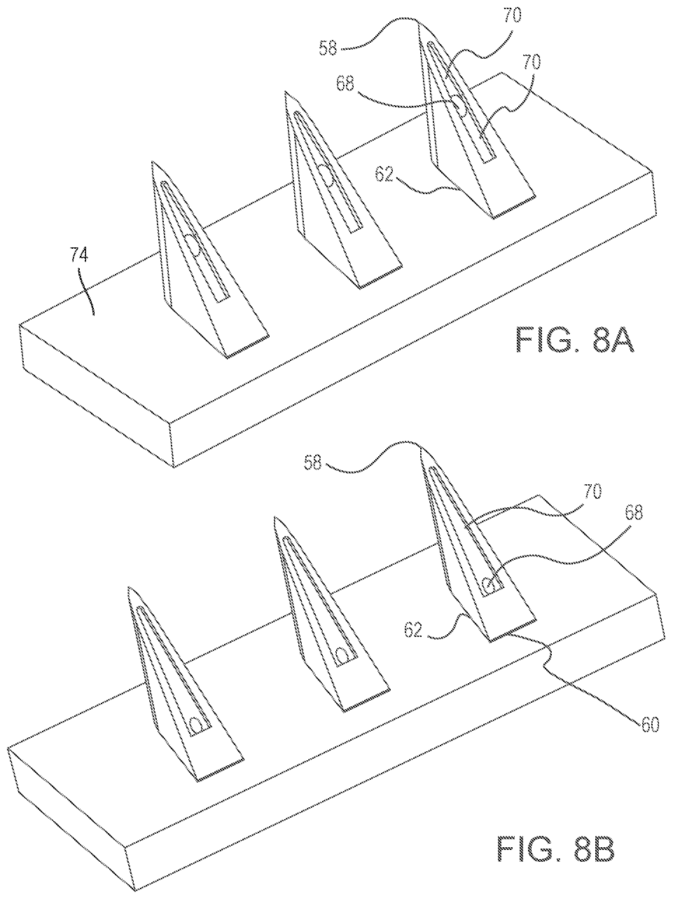

The depth of the channel 70 may be about 25 microns or more (e.g. 25-50 microns) in certain examples. The depth of the channel 70 may be or be less than 5 percent the height of the microneedle. While the depth of the channel 70 may be constant along the length of the channel 70, the depth of the channel 70 need not be constant along the length of the channel 70. Likewise, the width of the channel 70 need not be constant along the length of the channel 70 (see, e.g., FIG. 8B). The width of the channel 70 may be about 20-30 percent of the width of the distal side 60 of the microneedle at the narrowest point in the channel 70. In some embodiments, the width of the channel 70 may increase as distance to the distal side 60 decreases. In some embodiments, at its widest, the channel 70 may have a width which is 50% or more the width of the distal side 60.

Referring now also to FIG. 8A and FIG. 8B, in other examples, the channel 70 may extend from the location of the lumen 68 toward the tip or vertex 58 of the microneedle (see, e.g., FIG. 8B). Moreover, in some examples, the channel 70 may extend both toward the vertex 58 and toward the base 62 from the location of the lumen 68. That is, the channel 70 may include a portion on both sides of the lumen 68 (see, e.g., FIG. 8A). As shown, the lumen 68 may be located substantially centrally in the sloped face 72 of the microneedle. In such embodiments, a channel 70 may extend toward the distal side 60 of the base 62 and a channel 70 may extend toward the tip or vertex 58. In other embodiments, the lumen 68 may be positioned at (or near) an end of the channel 70 most proximal the base 62.

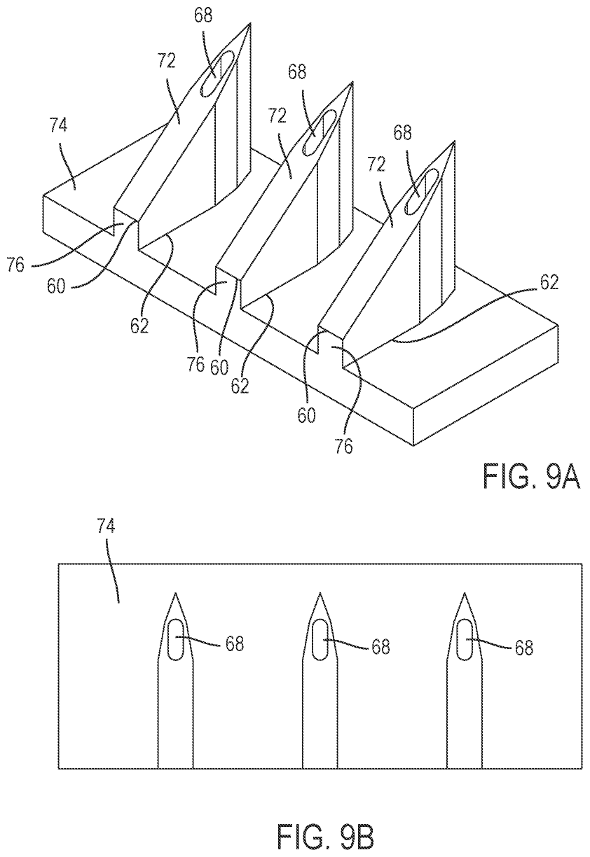

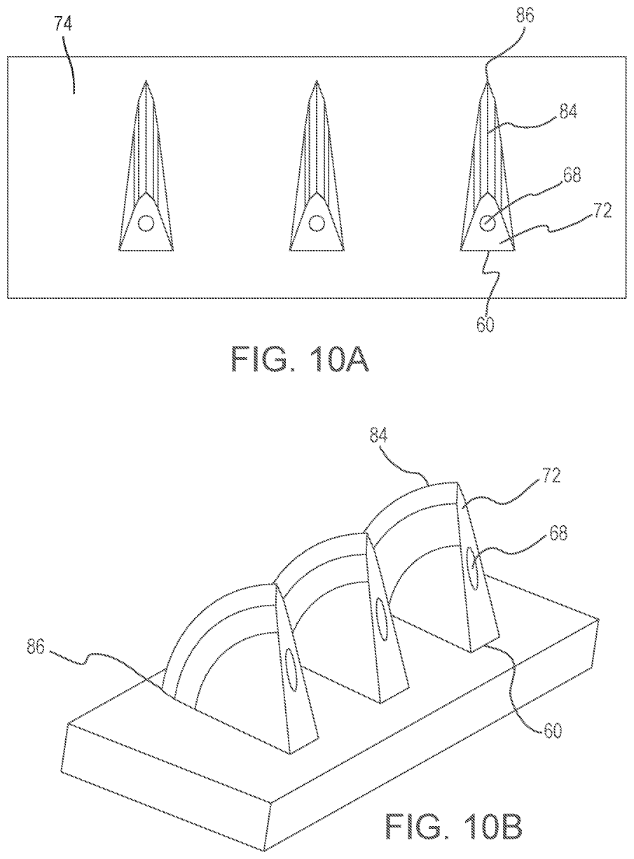

Referring now to FIGS. 9A-9B, views of a sharp bearing body 74 including a number of microneedles are shown. In certain embodiments, a channel 70 may not be included. Instead, a microneedle may include a flow lumen 68 with an elongate cross-section (at least at the outlet, see also FIGS. 10B & 11B). Microneedles with channels 70 and elongate lumens 68 are also possible. When in place within the patient, an elongate lumen 68 may be in fluid communication with, for example, multiple layers of skin. Thus, a thin and/or weak layer of skin may be easier to target when the microneedle is advanced into a patient. Elongate lumens 68 may also help to lower pressure required to inject. Such elongate flow lumens 68 may have any suitable cross-section. In some embodiments, the cross-section may be oval or elliptical. Alternatively, a lumen 68 with an obround cross-section may be used as is shown in FIGS. 9A-9B. Polygonal cross-sectional shapes may also be used, such as though not limited to rectangular, trapezoidal, triangular, etc. In certain examples, the length (in the direction of elongation) of the cross-section of the lumen 68 may be up to 100-200 microns or greater (though could be less in certain examples). Where elongate lumens 68 are included, the end of the lumen 68 most proximal the distal side 60 may be spaced from the distal side 60 by at least a certain distance. The spacing may be such that, the end of the lumen 68 most proximal the distal side 60 may be at least below the stratum corneum (and perhaps one or more of the stratum lucidum, stratum granulosum, stratum spinosum, and stratum basale) when the microneedle is inserted into the skin. In some embodiments, it may be disposed below the epidermis (e.g. in the basement membrane) or within the epidermis.

Still referring to FIGS. 9A-9B in certain embodiments, the sloped face 72 of a microneedle may not extend to the base 62 of a microneedle. There may, for example, be a vertical face 76 extending from the base 62 to the distal side 60 of a microneedle. Where a vertical face 76 is included, the vertical face 76 may be aligned with a side (e.g. distal side 60) of a sharp bearing body 74 and may form an extension thereof. Including such vertical faces 76 may aid in reducing the size of a sharp bearing body 74 and may aid in ensuring consistent fluid delivery into a target destination for certain microneedles. Though shown in relation to FIGS. 9A-9B, any of the microneedles shown herein may be arranged with vertical faces 76.

Additionally or in the alternative, a microneedle may include a depression 78. The depression 78 may include first and second opposing vertices 80, 82. In some embodiments the depression 78 may be (though need not necessarily be) a rounded depression or a concave depression, as shown in FIGS. 6A-6B. The depression 78 may have a maximum depth which places the depression 78 into fluid communication with the flow lumen 68 of the microneedle. The depression 78 may thus form a side port for the microneedle through which fluid may be delivered to the patient. The side port may be the only outlet of the microneedle or may be in addition to an outlet of the lumen in the sloped face 72 of the microneedle. When the microneedle is inserted into the skin surface, fluid transferred through a set 12 may be delivered to the patient, at least in part, by being pumped into the depression 78. The depression 78 may be formed, for example by cutting away material during manufacture of the microneedle or the depression 78 may be formed during a molding operation. Cutting away material may be accomplished by any known suitable process such as, for instance, etching (e.g. wet etching). In some embodiments, the depression 78 may be recessed in at least one side wall 64 or edge (e.g. where two side walls 64 join) of the microneedle. In the example shown in FIGS. 6A-6B, the depression 78 is formed in a substantially vertical back facing edge 66 of the microneedles which extends from the base 62 to the vertex 58. This may establish or increase a vertical void volume created by the microneedle as the skin is penetrated by the microneedle. That is, such a depression 78 may establish an open space in a patient into which fluid may be easily delivered from the microneedle. Positioning the depression 78 in the back facing edge 66 may provide a path of low resistance for a fluid to enter skin that the microneedle has penetrated. In embodiments wherein the microneedle includes at least one substantially vertical wall, the depression 78 may be recessed into a substantially vertical wall. In the example embodiment, the maximum depth of the depression 78 may be about 130% to 110% of the distance from the back facing edge 66 to the flow lumen 68.

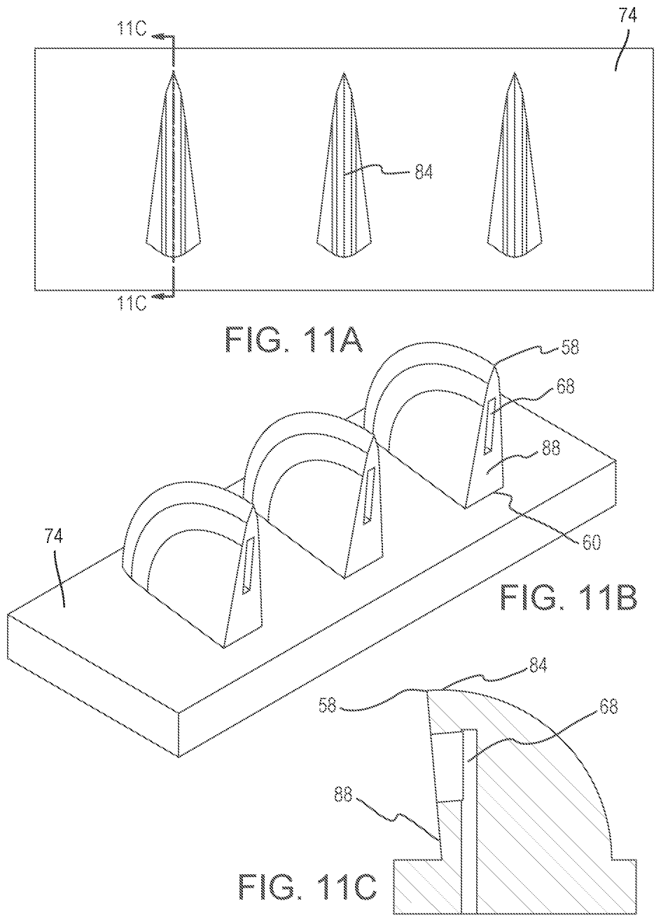

In certain examples, and referring now to FIG. 10A and FIG. 10B, a microneedle may include a sloped face 72 to which a lumen 68 extending through the microneedle extends. A microneedle may also include a rounded blade edge 84. In the example, the rounded blade edge 84 extends from a point 86 opposite the distal side 60 and extends in an arcuate path to the vertex or tip 58 of the microneedle. In the example, the rounded blade edge 84 includes a double bevel, though other bevel types may be used. The rounded blade edge 84 may arc at a constant radius or a variable radius. The rounded blade edge 84 may have an arc measure of less than 90° or, in certain examples, greater than 90° (see, e.g., FIG. 11A-11C). The rounded blade edge 84 may aid in introduction of a microneedle into skin when the microneedle is inserted at certain angles or over a variety of different angles.

In yet another embodiment, and referring now to FIGS. 11A-11C, a microneedle may include a rounded blade edge 84 and a lumen outlet face 88. The lumen 68 may extend through the microneedle to the lumen outlet face 88 and may not be formed in a straight line through the microneedle. The lumen outlet face 88 may be angled from the vertex 58 to the distal side 60 so as to form an undercut. The distal edge 60 may be disposed such that a plane perpendicular to the base 62 passing through the distal edge 60 may also pass through the rounded or arcuate blade edge 84. Additionally, the outlet of the flow lumen 68 in the lumen outlet face 88 may be disposed such that a plane or all planes perpendicular to the base 62 and passing through the outlet of the flow lumen 68 may also pass through the blade edge 84. This need not be true in all embodiments (see, e.g., FIGS. 10A-10B). As a microneedle of the variety shown in FIGS. 11A-11C is inserted, a vertical void space may be created due to the undercut. This may provide a low resistance pathway for fluid injection. Additionally, the undercut may help to mitigate potential for the lumen 68 to become obstructed by skin as the microneedle is inserted into a patient or as the delivery occurs.

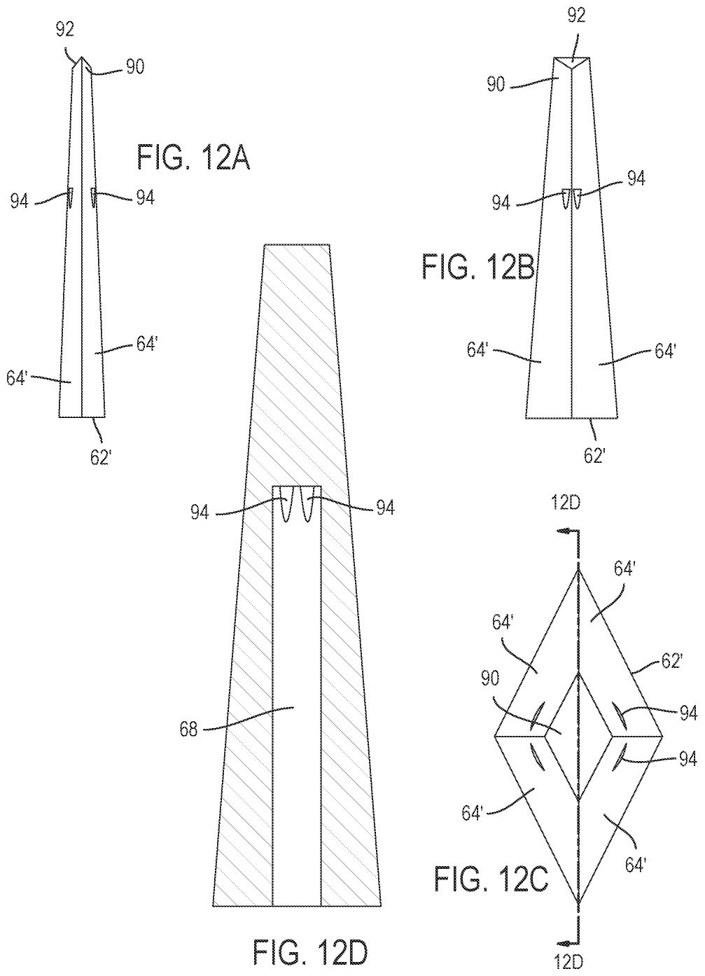

In still other embodiments and referring now to FIGS. 12A-12D, the access member(s) 16 may be or include a microneedle which has a shape with a high aspect ratio. In some embodiments, microneedles may be obelisk shaped. Such microneedles may be included in an array such as any array described herein. Where obelisk type microneedles are used, the microneedles may include a base 62′. The base 62′ may be any desired round or polygonal shape. For purposes of example, FIGS. 12A-12D depict a base 62′ which is a quadrilateral or rhombus. The example microneedle includes a set of sidewalls 64′ which extend from the base 62′ to an end region 90 of the microneedle. The sidewalls 64′ may be disposed at an angle which is not perpendicular to the base 62′. Thus the microneedle may taper so as to have a smaller cross-sectional area as distance from the base 62′ increases. A portion of the microneedle most distal to the base 62′ may include a beveled tip 92. Such a tip 92 may facilitate puncture of the skin and may aid in increasing the robustness of the end region 90. Any suitable bevel such as a single or double bevel may be used.

In embodiments of microneedles which are obelisk shaped, the microneedles may include at least one side port 94 which may serve as an outlet for that microneedle. Such side port(s) 94 may be difficult to block off with tissue which that may become compressed during insertion of the microneedle into a patient. In the example embodiment, a lumen 68 may extend through the base 62′ of the microneedle and have a terminal end which is more proximal the end region 90 than the base 62′. The lumen 68 may be of relatively constant cross-section. The taper of the sidewalls 64′ may be such that the terminal end of the lumen 68 is wider than portions of the cross-section of the corresponding region of the microneedle. Thus, the lumen 68 may form openings in the sidewalls 64′ which may serve as the side ports 94. In various examples, the lumen 68 may be centrally disposed yielding symmetrical side ports 94. In alternative embodiments, the lumen 68 need not be centrally disposed and the side ports 94 may not be symmetrical.

Microneedles and features thereof may be manufactured in one or more of, though are not limited to, a molding process, etching process, ablative process (e.g. laser ablation), or a material additive process (e.g. 3D printed). In various embodiments, it may be desirable that microneedles be constructed of a biocompatible, non-ductile, high Young's modulus material with an indentation hardness sufficient to allow penetration into skin without breakage.

Sets 12 including microneedles such as any of those described herein may be painless or nearly pain free is apply to a patient. This may make such sets user preferable over other types of infusion sets. This may be particularly true of certain patient populations such as patients with juvenile diabetes. Additionally, such sets 12 may be less complicated to apply. A set 12 including microneedles may be ready or substantially ready (e.g. an adhesive backing may need to be removed) for application when removed from a package. Thus, no inserter used to assisting in puncturing the skin and placing an infusion set may be needed. Sets 12 including microneedles may facilitate easy site changes without the need for a user to carry additional, relatively bulky components such as an inserter. Moreover, as an inserter (which may typically be a single use disposable) may be omitted, patient financial burden associated with frequent site changes may be mitigated. This may help increase patient compliance with prescribed site change schedules or may allow for site changes to performed more frequently (e.g. daily).

Referring again to FIGS. 4A-4B, an example set 12 may include a main body 52. The main body 52 may be a deformable body which may transition from a storage state (see FIG. 4A) to a delivery state (see FIG. 4B). In certain examples, this transition may be reversible, though in other embodiments the transition may result in a permanent change in the main body 52 and/or another part of the set 12. For example, once transitioned to the delivery state, at least a portion of the main body 52 may plastically deform such that it is permanently distorted and may not be returned to the storage state. Destruction of a portion of the main body 52 or a portion of a set 12 engaged to the main body 52 may be required to remove the set 12 from a patient rendering the set 12 inoperative. Where a permanent change is engendered upon transition to the delivery state, this permanent change may inhibit reuse as well as provide a user perceptible (e.g. visual) indication that a set 12 has been used. An indication that the transition has occurred may also be generated by a set 12. For instance, an audible or tactile indication may be generated upon transition from the storage state to the delivery state.

In various examples, transition of the set 12 from the storage state to the delivery state may be accomplished via bending, pivoting, or deformation of one or more regions of the main body 52. In certain examples, the main body 20 may be or include a bi-stable element which may have a first stable state which corresponds to the storage state and a second stable state which corresponds to the delivery state. The main body 52 may for example substantially or partially invert (e.g. convex to concave) in shape or have one or more invertible regions which at least partially invert when the set 12 is transitioned from the storage state to the delivery state. The transition may be affected via application of force throughout the entire transition. Alternatively, the transition may only require application of force throughout a portion of the transition. For example, in some embodiments a triggering force may be applied to initiate the transition and the transition may subsequently complete in the absence of any external application of force. For example, after application of the triggering force, the transition may be characterized by a snap-through buckling via which the main body 52 rapidly shifts into the delivery state.

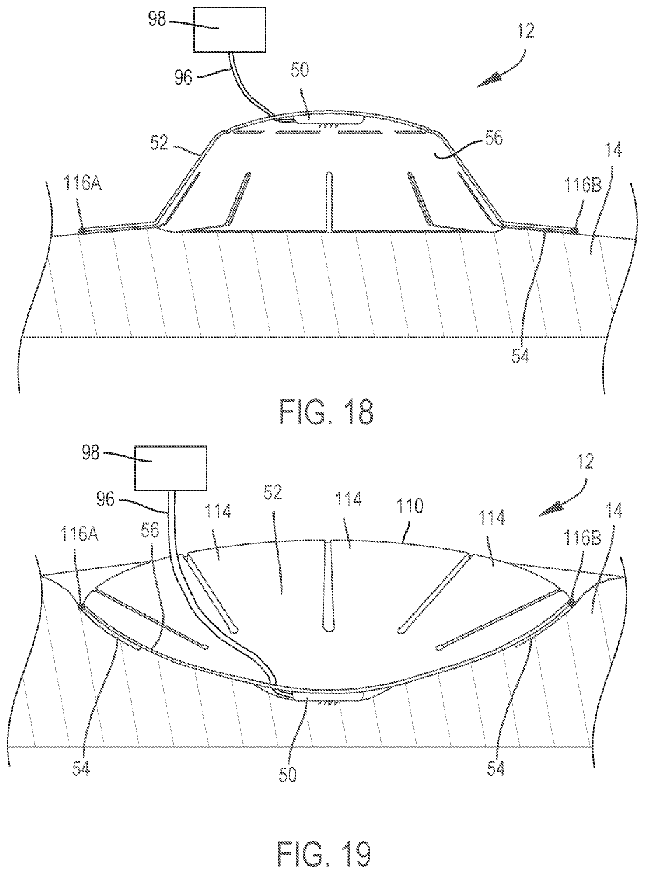

The main body 52 may be at least partially covered with adhesive 54 over a first face 56 of the main body 52. The adhesive 54 may serve to couple the main body 52 to a skin surface at an infusion site on a patient. Thus, the first face 56 may be a skin adjacent face or proximal (proximal and distal defined in relation to a patient) face of the main body 52. The main body 52 may be adhered to the skin when the main body 52 is in the storage state and then may be transitioned to the delivery state. As the transition occurs, at least two adhesive bearing portions of the main body 52 may be displaced with respect to one another so as to stretch or spread a surface anchored to the main body 52 via the adhesive 54. As these portions may be adhered to the skin surface, the skin may be stretched as the adhesive bearing portions are displaced with respect to one another. This may be desirable as the skin may be rendered taught facilitating piercing of the skin by the access members 16 as the main body 52 transitions to the delivery state. In certain examples, the adhesive 54 bearing portions may be disposed, for example, in opposition to one another. The displacement of the two adhesive 54 bearing portions may increase the distance between or spread apart the two adhesive 54 bearing portions. In other embodiments, the distance between the two adhesive 54 bearing portions may not increase or may even decrease while still causing stretching of the skin surface. This may for example occur if the transition causes a flat patch of skin to be pulled around a curve or contour of the main body 52 (see, e.g., FIGS. 18-19). A displacement of adhesive 54 bearing portions with respect to one another that results stretching of the adhered skin (regardless of any positive or negative change in distance between the adhesive 54 bearing portions) may be referred to as a spreading displacement. Two adhesive 54 bearing portions which have been so displaced may be referred to as being spreadingly displaced.

Transition of the main body 52 to the delivery state may also result in a proximal displacement or lowering of any access member(s) 16 toward and into the skin. In embodiments described herein, the access member(s) 16 may be covered prior to use.

Referring now to FIGS. 13-15, an exemplary set 12 is depicted. The example set 12 is shown in a storage state in FIGS. 13-15. As shown, the set 12 may include a main body 52 and a body 50 to which access member(s) 16 may be coupled. The access member(s) 16 may be included on a sharp bearing body 74 which may be coupled to body 50. A conduit 96 (e.g. microbore tubing) may also couple to the body 50 and a flow path in communication with the access member(s) 16 may be included in the body 50. The conduit 96 may be plumbed into communication with this flow path through a portion of the main body 52. The conduit 96 may include a coupling 98 to which a connector 26 (e.g. a luer connector, see. e.g. FIG. 1 or a connector 26 such as that shown in FIG. 43) may be coupled. A coupling 98 may be associated with a check valve to inhibit flow of fluid when not connected to a fluid supply (e.g. connector 26 from an infusion device 18.

The main body 52 of the set 12 may have a round (e.g. circular) foot print and may include a central region 100 and a peripheral region 102. The central region 100 may be a raised region of the main body 52 and the peripheral region 102 may be a substantially flat region of the main body 52 which surrounds the central region 100. The thickness of the main body 52 may be substantially uniform over the entirety of the main body 52. The main body 52 may be formed as a thin sheet or disc of material which may be thermoformed to create the raised central region 100 and flat peripheral region 102.

Alternatively, the main body 52 may be injection molded and the raised central region 100 and flat peripheral region 102 may be formed in the molding operation. In various embodiments where sets 12 are or may be injection molded, the main body 52 may be injection molded so as to be in the storage state or in the delivery state. The main body 52 may transition more easily into the state in which it was molded from the opposite state. Thus, to lower the effort needed to transition a set 12 from a storage state to a delivery state, it may be desirable to mold the main body 52 of the set 12 in its delivery state configuration. During assembly of a set 12, the main body 52 may be brought into its storage state configuration and remain in that configuration until use.

The central region 100 may be domed and the domed shape may establish a receptacle 104 on the proximal side of the main body 52 within which the body 50 may be disposed. The body 50 may be coupled within the receptacle 104 via adhesive or in another suitable manner. The central region 100 may also include a series of fenestrations 106 which may form a fenestrated ring in the central region 100. In the example, the fenestrations 106 are evenly spaced from one another and arranged in a circle which is generally coaxial with the center of the central region 100. In alternative embodiments, fenestrations 106 may be irregularly spaced or omitted. Additionally, in some embodiments, the fenestrations 106 may instead be replaced with thinned regions or a ring where the material of the main body 100 is thinned.

The main body 52 may include a number of slots 108. The slots 108 may extend from a peripheral edge 110 of the main body 52 toward a center or midpoint of the main body 52. In the example embodiment, the slots 108 extend in a radial direction. The slots 108 may extend through the entirety of the peripheral region 102. In some embodiments, and as shown, the slots 108 may additionally extend though at least a portion of the central region 100 as well. The fenestrations 106 in the central region 100 may be disposed radially inward of the terminus 112 of each of the slots 108. The main body 52 may thus include a central region 100 which is circumscribed by a number of petal members 114 which are spaced apart via the slots 108.

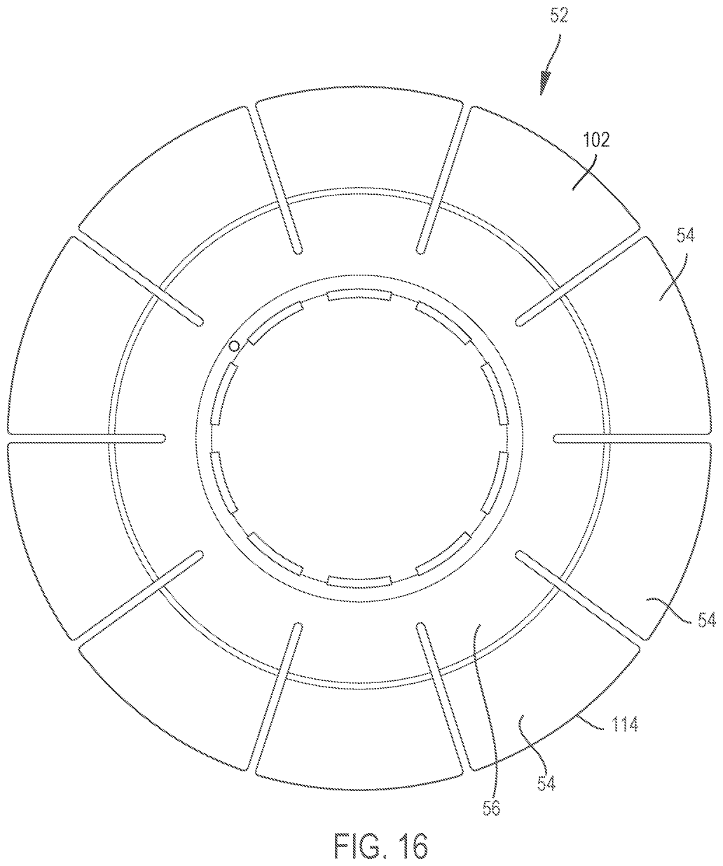

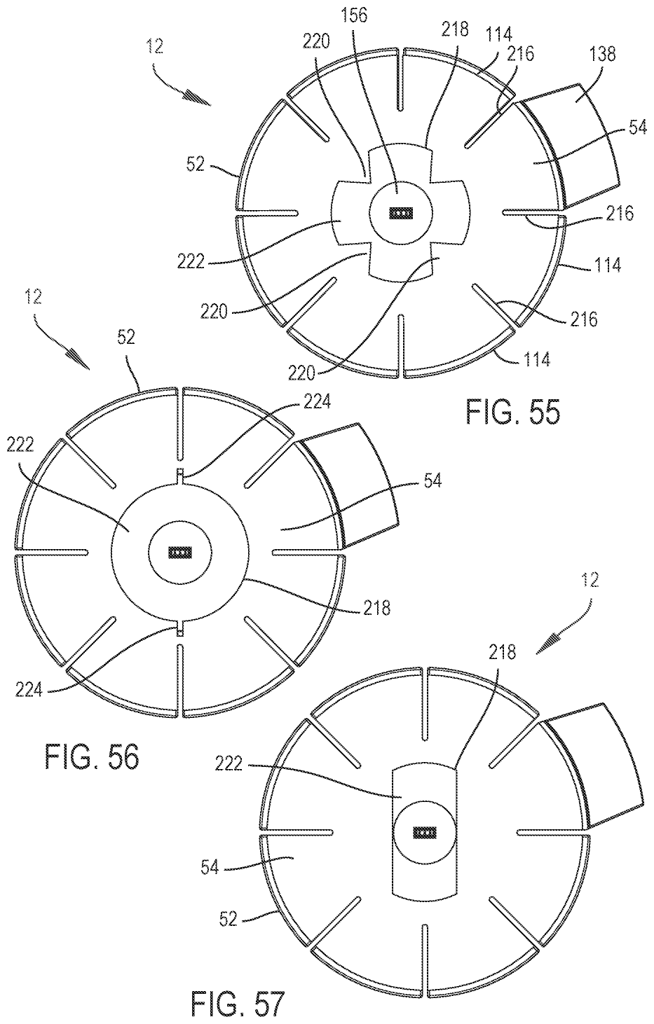

Referring now to FIG. 16, a plan view of the proximal face 56 of an example main body 54 is depicted. As shown, adhesive 54 may be included on at least a portion of the proximal face 56. The adhesive 54 may be a skin compatible adhesive and may serve to couple a set 14 to a skin 14 surface at an infusion site. In the example embodiments, adhesive 54 may be included on the peripheral region 102 of the main body 52. Though adhesive 54 is shown covering the entire surface of each of the petal members 114 in the peripheral region 102, other embodiments may differ. For example, only certain petal members 114 may include the adhesive 54. In such embodiments, adhesive 54 may be included on at least one pair of oppositely disposed (e.g. diametrically opposed in the example embodiment) petal members 114. Only a portion (e.g. a majority of the surface area) of each petal member 114 included in the peripheral region 102 may be covered with adhesive 54 in some examples. Alternatively or additionally, the adhesive 54 may differ from petal member 114 to petal member 114. Certain petal members 14 may be covered with a more aggressive adhesive 54 while other petal members 114 may be covered with a less aggressive adhesive 54. Additional adhesive members 54 are described elsewhere herein (see, e.g., FIGS. 55-57) and may be used on a set 12.

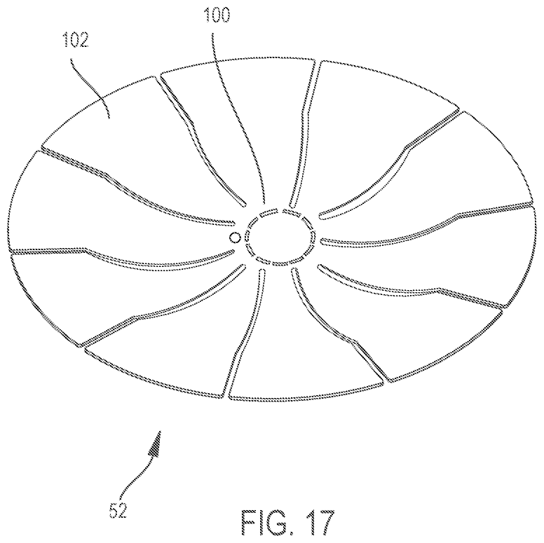

Referring now to FIG. 17, a conceptual representation of a main body 52 of a set 12 is depicted in a delivery state. In the delivery state, at least the central region 100 of the main body 52 may substantially invert. The fenestrations 106 may facilitate this inversion by helping to allow for increased deflection of the main body 52 at the fenestrations 106. Thus, in place of a convex dome-like shape, the center region 100 of the main body 52 may take on a concave shape. As the peripheral region 102 is coupled to the center region 100, the peripheral region 102 may displace as a result of the inverting of the center region 100. In the example embodiment, the entire main body 52 takes on a bowl shape when transitioned to the delivery state. The peripheral region 102 may also spreadingly displace for at least a portion of the transition. The slots 108 in the main body 52 may help to facilitate spreading displacement of the petal members 114 as the transition takes place, thereby enhancing stretching of the skin of user.

The main body 52 may be a bi-stable element or include at least one bi-stable region which may be stable in both the storage state and the delivery state. When an axial load is applied on the central region 100 and the main body 52 is in the storage state, the main body 52 may deform into an unstable state. The main body 52 may then exhibit a snap through buckling action which rapidly shifts the main body 52 into the stable delivery state similar to that shown in FIG. 17. Thus, only a triggering force may be applied to initiate the transition. The rest of the shift between the storage and delivery state may be caused by the snap through phenomenon.

FIG. 18 depicts an exemplary set 12 in a storage state and adhered to skin 14 via adhesive 54 on a proximal face 56 of the main body 52. FIG. 19 is a conceptual representation depicting an example set 12 in the delivery state. As shown, the set 12 may be applied to the skin 14 in the storage state. The set 12 may then be transitioned to the delivery state. A spreading displacement of opposed petal members 114 of the main body 52 may occur as the transition transpires.

Two opposing points 116A, B disposed at the peripheral edge 110 of the proximal surface 56 are shown in FIGS. 18-19. When the set 12 is in the storage state (FIG. 18), the shortest distance between the opposing points 116A, B is a straight line which does not pass through the proximal surface 56. This straight line is roughly parallel to the surface of the skin 14. In the delivery state, however, the shortest distance between the opposing points 116A, B is a straight line which passes through the proximal surface 56. As the skin 14 is fixed to the main body 52 via the adhesive 54 and cannot pass through the main body 52, the skin 14 may be forced to conform to the curvature of the proximal surface 56. Thus, the length of the skin 14 surface between the two points 116A, B when the set 12 is in the delivery state may be greater than the length of the skin 14 surface between the points 116A, B when the set 12 is in the storage state. The skin 14 may be placed under tension and stretched to accommodate this change in length. This stretching may, in turn, aid in facilitating puncture of the skin 14 by the access member(s) 16.

Due to the elasticity of the skin 14, the skin 14 may exert a restoring force against the proximal surface 56 of the main body 52 as it attempts to revert to an unstretched state. The main body 52 may resist this restoring force and retain its bowl shape. The body 50, however, may be compressed between the skin 14 and the main body 52. This may aid in ensuring the access member(s) 16 puncture the skin 14 and enter fluid communication with a target delivery destination in the patient.

As mentioned above, in certain examples, some petal members 114 may not include adhesive 54 regions or may have a proximal surface 56 which is at least partially covered in adhesive 54 that is less aggressive than adhesive 54 of on other petal members 114. In embodiments where some petal members 114 are devoid of adhesive 54, this may help to limit stretching of the skin 14. Likewise, petal members 114 with less aggressive adhesive 52 may release the patches of skin 14 to which they are affixed if force needed to stretch the skin 14 exceeds a threshold. The petal members 114 themselves may also be constructed such that at least one of the petal members 114 includes a relief region (e.g. a thin or narrow region). For example, if force needed to stretch the skin 14 is above a threshold, one of more of the petal members 114 may bend or buckle at the relief region to relieve some of the tension on the skin 14.

This may be desirable as it may help to mitigate potential discomfort during wear of a set 12 due to excessive tensioning of the skin 14. Additionally, this may be helpful in certain patient populations as skin characteristics vary significantly with age, hydration state, lifestyle (sun exposure, nutrition), etc. It may be desirable that slacker or looser skin be stretched to a greater degree than highly elastic skin. Thus, instead of providing a variety of sets 12 with different adhesives 54 targeted at specified patient populations, a set 12 may be made in a more universal manner.

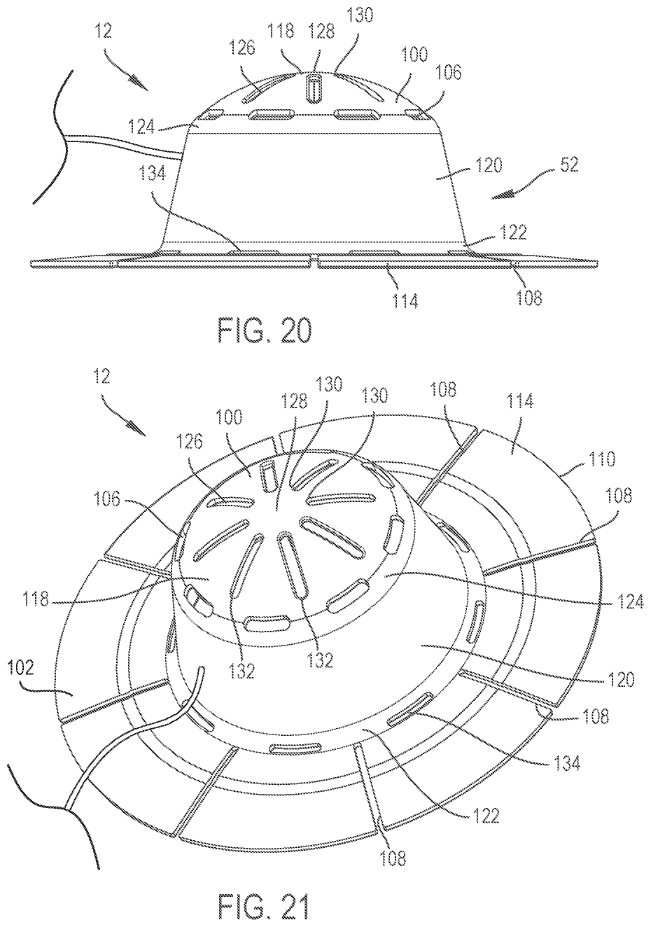

With reference to FIGS. 20-21, in another embodiment, an example set 12 may include a central region 100 having a top surface 118 and a supporting structure 120 integral with the top surface 118. The supporting structure 120 may have a round, e.g., substantially circular, base 122. The peripheral region 102 may be roughly annular shaped and may include an inner perimeter coincident with the base 122 and an outer perimeter, or peripheral edge 110. The set 12 may be formed by injection molding. The top surface 118 may have a round footprint, e.g., of roughly circular shape, and may be convex, forming a dome shape. The top surface 118 may have a periphery 124. The top surface 118 may include slots 126. The slots 126 may be cutouts, apertures, holes, openings, or voids in various embodiments. The slots 126 may help the set 12 to transition from the storage state to the delivery state with reduced pressure from above. The slots 126 may extend radially with respect to a center point 128 of the top surface 118 such that their respective first endpoints 130 surround a region including the center point 128 of the top surface 118 and their respective second endpoints 132 may each terminate a distance (e.g., the slots 126 may each terminate the same distance) from the periphery 124 of the top surface 118. In embodiments including slots 126, the slots 126 may be disposed at regular angular increments (though need not be). In embodiments described herein including slots 126, the slots 126 may (though need not necessarily be) each be of the same length.

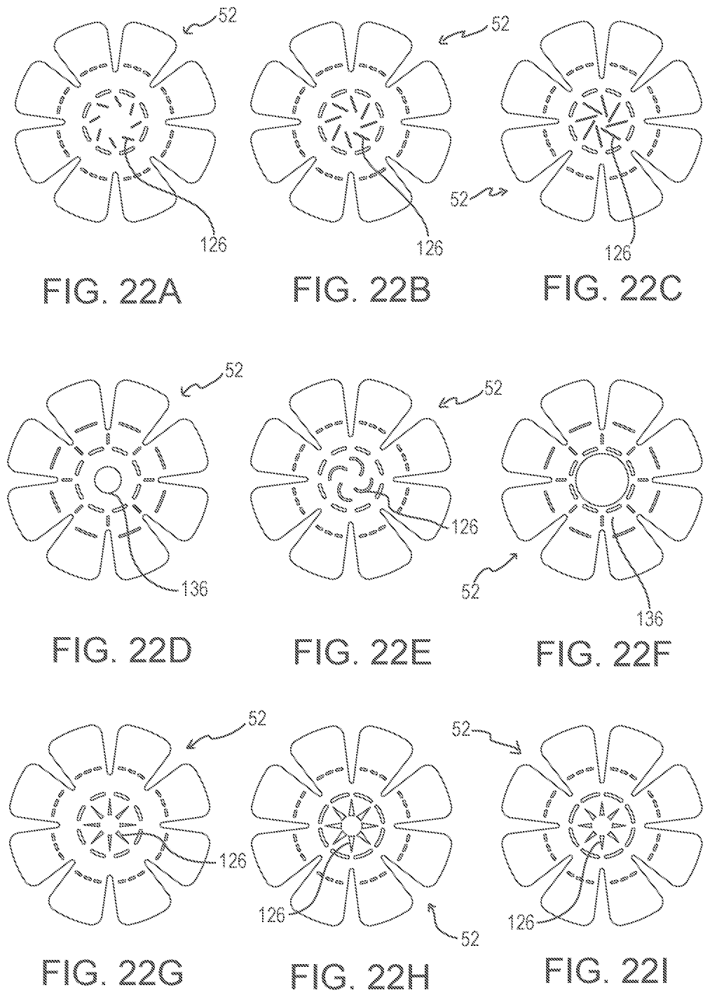

Referring now to FIGS. 22A-22I, a variety of different main body 52 embodiments are depicted. The exemplary main bodies 52 are shown in a flat state and may be thermoformed into a configuration such as that shown in, for example FIG. 20. Though thermoformable main bodies 52 are depicted, the features described in relation to thermoformed main bodies 52 may be included in main bodies 52 which are manufactured in any desired manner. As shown in FIGS. 22A-22I, the slots 126 may be provided in a number of different formats. Additionally, in some embodiments, slots 126 may not be included.

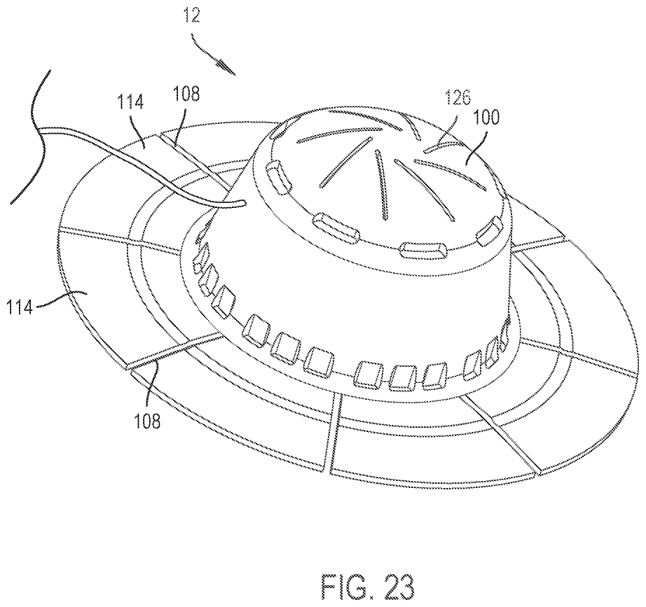

In some embodiments, and as also shown in FIG. 23, the slots 126 could be disposed such that they do not extend radially with respect to the center point 128. For example, the slots 126 may each extend at a common angle with respect to a radial direction. In such embodiments, slots 126 may be evenly spaced about the top surface 118 and may each be of the same length. In other embodiments, the slots 126 may not all extend at a common angle to the radial direction. At least one of the slots 126 (and perhaps all) may be disposed at a different angle to the radial direction. In some embodiments, the slots 126 may be relatively short, positioned about the periphery 124 of the top surface 118, and may be disposed within an outer region of the top surface 118 (see, e.g., FIG. 22A). In other embodiments, slots 126 may extend across an outer region and intermediate region of the top surface 118 (see, e.g., FIG. 22B). In still other embodiments, slots 126 may extend from the outer region of the top surface and into a center region of the top surface 118 (see, e.g., FIG. 22C). The angled slots 126 may aid in lowering the amount of pressure needed to transition a set 12 from a storage state to a delivery state. Positioning the slots 126 at a sharper angle with respect to the radial direction may generally lower this pressure. The width of the slots 126 may slightly decrease during at least a portion of the transition from the storage state to the delivery state.

In other embodiments, and referring primarily to FIG. 22E, at least one of the slots 126 may have a curvature. The curvature may be defined by a constant or variable radius. The curvature may only be present over a segment of the slot 126. In alternative embodiments, a slot 2126 may include two or more sections which are angled with respect to one another. In the example embodiment shown in FIG. 22E, four curved slots 126 are shown and are spaced apart at even angular increments. The slots 126 are arcuate and include a first end 130 and second end 132. Each example slot 126 is oriented so as to initially begin extending in a first direction from the first end 130 and curve so as to extend in a second direction as the slot 126 reaches the second end 132. The second direction may be closer to perpendicular (or may be perpendicular) to the radial direction than the first direction.

In some examples, and referring now primarily to FIGS. 22D and 22F, the top surface may not include a slot 126 or slots 126 but may instead include at least one aperture 136. In the examples shown, the aperture 136 is disposed centrally within the top surface 118. The aperture 136 may extend over a minority or a majority of the top surface 118. In some embodiments, the aperture 136 may encompass nearly the entirety of the top surface 118.

As shown exemplarily in FIGS. 22D and 22F, slots 126 may also be included in other regions of a main body 52. In the example embodiment, the region of the main body 52 which would become the supporting structure 120 (when the main body 52 is thermoformed) includes slots 2126. These slots 126 may be straight, curved, angled (with respect to the radial dimension) or some mix thereof as with various top surface 118 slot 126 patterns described herein. As shown, the slots 126 are spaced at regular angular intervals and are spaced between petal members 114 of the main bodies 52.

In still other embodiments, the width of one or more of the slots 126 may vary over the length of that slot 126. A number of embodiments including variable width slots 126 are depicted in FIGS. 22G-22I. The slots 126 may change in width in a continuous manner and may terminate with a pointed first or second end 130, 132. Variable width slots 126 may extend along a radial direction, though need not necessarily do so in all embodiments. In the example embodiments, each of the slots 126 are widest proximal to the center point 126 of the top surface 118 and continuously decrease in width as the slot 126 extend distally toward the periphery 124 of the top surface 118. Thus, each of the top surfaces 118 depicted in FIGS. 22G-22I have a sunburst type pattern of slots 126. In other embodiments, the slots 126 need not necessarily continuously increase or decrease in width from one end to the other.

Still referring to FIG. 23, the central region 100 may be monolithically formed with the petal members 114 comprising the regions between respective pairs of slots 108 (see also FIGS. 13-19 and the examples and embodiments described above with respect thereto). The supporting structure 120 may extend upward from the petal members 114 at a 90° angle or an angle greater than 90 degrees, e.g., 100-105 degrees, although the angle measure need not be limited to a range. The distance (vertically) from the base 122 of the supporting structure 120 to the periphery 124 of the top surface 118 may be long enough to provide a receptacle in the central region 100 for a body 50. The slots 108 may extend from the peripheral edge 110 of the set 12 to the base 122 of the supporting structure 120, but may terminate at the base 122 and not extend into the supporting structure 120 itself. In such embodiments, rather than the entire central region 100 substantially inverting when pressure is applied from the top (e.g., by a finger), only the top surface 118 may invert, taking on a concave shape in the delivery state. The supporting structure 120 may in some embodiments include fenestrations 134 evenly spaced about the base 122. The fenestrations 134 may facilitate manufacturing of the set 12 in embodiments in which the main body 52 is thermoformed.

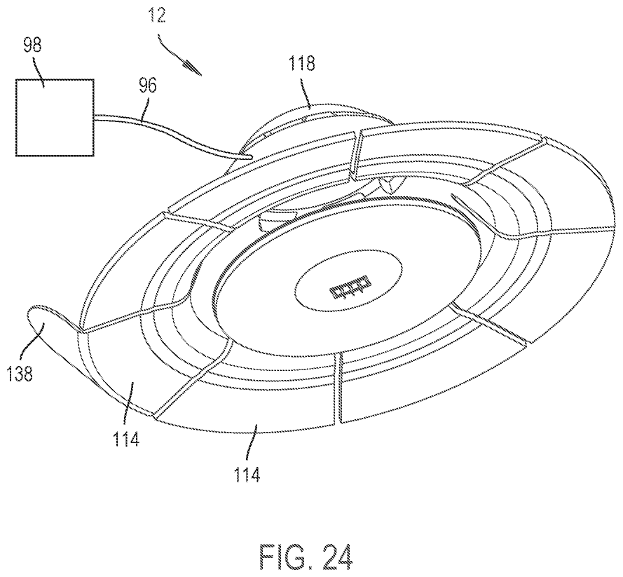

In some embodiments and as shown in one example in FIG. 24, at least one of the petal members 114 may be made of an extended length such that an outward end of the petal member 114 may be operated by a patient or health care provider as a pull tab 138. The pull tab 138 may be grasped by a user to remove the set 12 from the skin 14 (see, e.g., FIG. 1) after use. The pull tab 138 may be of any suitable shape. In an example, the pull tab 138 is approximately semicircular in shape, with a first, rounded end and a second end opposite the first end, attached to a petal member 114. The second end may be attached or formed integral with the petal member 114 by injection molding or any other known technique. Though the pull 138 tab is shown extending upward toward the top surface 118, in other embodiments, the pull tab 138 may be generally perpendicular to a height dimension of the set 12 in other examples. Thus the pull tab 138 may lay substantially flat against the skin 14 and be oriented generally parallel to the skin 14 when the set is being worn by a user. This may help to minimize opportunity for the pull tab 138 to catch or snag on clothing or other items.

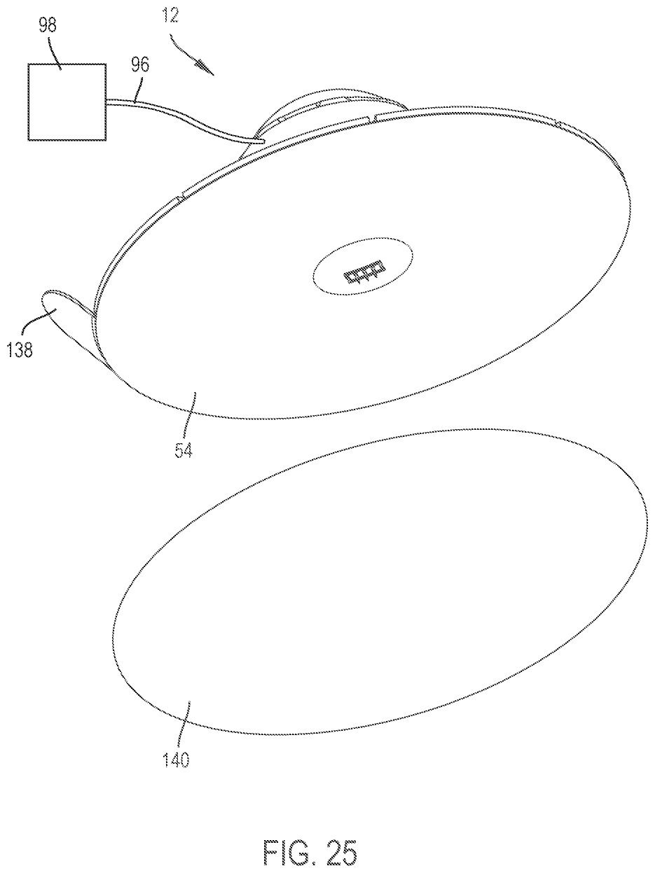

Referring now to FIG. 25, a release liner 140 which covers adhesive 54 on the set 12 may be removed from the bottom of the set 12 before the set 12 is applied to the skin 14 (see, e.g., FIG. 1) surface. A release liner 140 may be removed in a manner similar to how a release liner is peeled from a bandage before application to skin. An example set 12 having a pull tab 138 and including release liner 140 and adhesive 54 is depicted in FIG. 25. The release liner 140 is exploded away from the adhesive 54 for illustrative purposes.

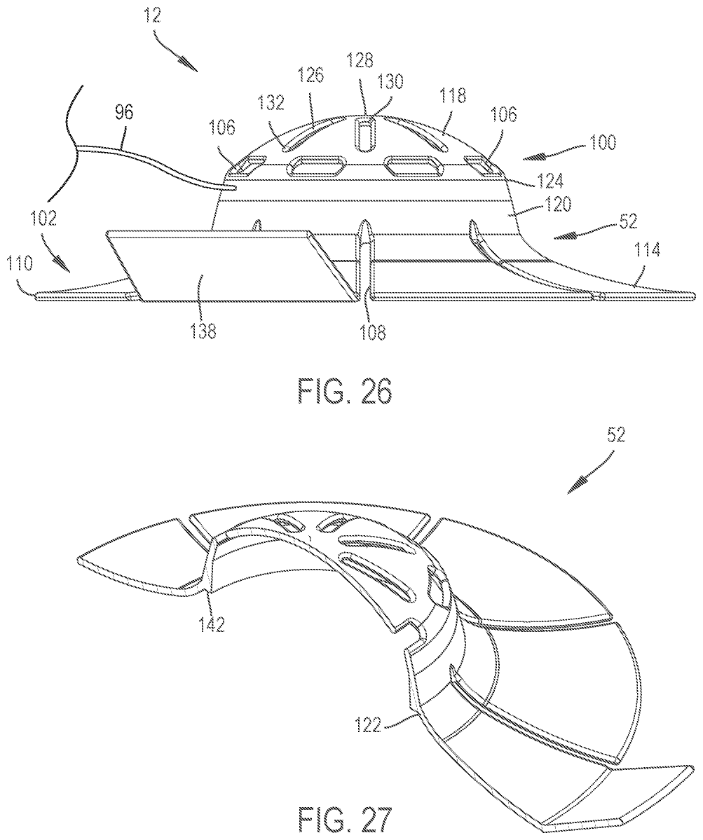

With reference to FIGS. 26-27 and FIGS. 28A-28B, in some embodiments, sets 12 may include a central region 100 which is roughly thimble, or dome, shaped but has a relatively shorter height compared to certain other embodiments described herein. The distance (vertically) from the base 122 to the periphery 124 of the top surface 118 may be relatively shorter. In some embodiments, the aforementioned distance may be approximately 0.15 inches.

Additionally or in the alternative, the peripheral region 102 may not be a substantially flat annular shape. The peripheral region 102 may be defined by curved petal members 114 that continue in a downward direction such that their peripheral edge 110 is spaced from the plane of the base 122 of the supporting structure 120 (e.g. about the same or less than the distance from the base 122 to the periphery 124 of the top surface 118). The peripheral edge 110 may be disposed along a plane which is more distal to the periphery 124 of the top surface 118 than the base 122. As depicted in FIG. 26, the exemplary set is shown in the storage state. The set 12 may include slots 108 which may be disposed between the petal members 114 like other set 12 embodiments described herein. An adhesive 54 (see, e.g., FIG. 25) may be affixed to at least a part of at least two of the petal members 114.

As best shown in FIG. 27, a perspective cross-sectional view of a main body 52 of an example set 12, the main body 52 may include an interior ridge 142. The ridge 142 may be disposed at the base 122 of the supporting structure 120. The supporting structure 120 may be thickened in a region near the base 122 so as to create the ridge 142. This may allow for the ridge 142 to be formed easily in, for example, an injection molding operation which forms the rest of a main body 52. This may also provide extra rigidity to the supporting structure 120. The ridge 142 may provide a step, ledge, or other mounting surface upon which a portion of a body 50 of a set 12 may be mounted. Such a ridge 142 may be included in any of the set 12 embodiments described herein. Bodies 50 and ridges 142 are further described elsewhere in the specification.

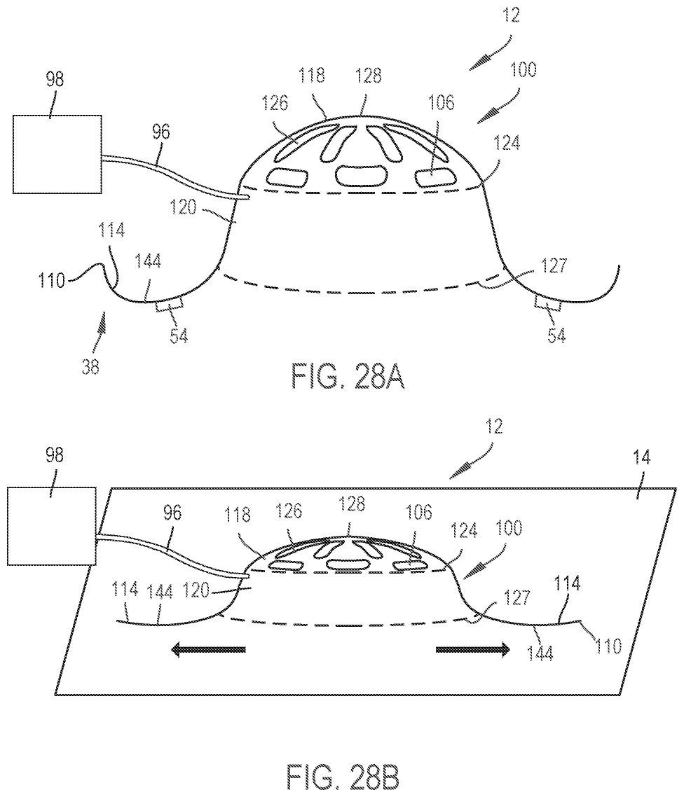

Referring now primarily to FIGS. 28A-28B, two conceptual representations of a set 12 transitioning from a storage state to a delivery state are shown. When the set 12 is affixed to the skin with an adhesive 54 and pressure is applied to the set 12 from above, e.g., by a user's fingertip, the set 12 may transition to a delivery state. When the petal members 114 are pushed against the surface of the skin, the petal members 114 may spreadingly displace outward and the skin 14 and/or patient's body may force at least a portion of the petal members 114 to curl upward. In turn, this may cause the skin to stretch as parts of opposing petal members 114, each affixed to the skin surface by adhesive 54 (only shown in FIG. 28A), move apart from one another or spreadingly displace. As the set 12 transitions to a delivery state, at least a portion of each of the curved petal members 114 may curve further or with a tighter radius of curvature. When a delivery state is reached, the curvature of the petal members 114 may be such that they may extend from the base 122 to an inflection point 144. The inflection point 144 may fall in a plane spaced from that of the base 122 and in such embodiments may also be referred to as a lowest point. In such embodiments, the lowest point 144 may be in a plane more distal to the periphery 124 of the top surface 118 than the base 122. From the inflection point 144, the petal members 114 may curve back upward so as to become increasingly more proximal to the plane in which the periphery 124 of the top surface 118 is disposed. The peripheral edge 110 of the petal members 114 may, for example, be disposed at a point at or above (more proximal the plane of the periphery 124 of the top surface 118) the plane of the base 122. The petal members 114 may, though need not necessarily, each have a constant radius of curvature from the inflection point 144 to the peripheral edge 110. The constant radii curvature back upward may enhance capability of the petal members 114 to curl upward. This may in turn enhance stretching of the skin of the user as points 144 on opposing petal members 114 (each affixed to the skin by adhesive 54) spreadingly displace. As mentioned elsewhere herein, the top surface 118 of the main body 54 may also invert as the set 12 is transitioned to the delivery state.

Still referring to FIGS. 26-28B, in some embodiments, the supporting structure 122 may not include fenestrations 134 (see, e.g. FIG. 20) evenly spaced around the base 122. The main body 52 may be manufactured by injection molding. Those of skill would readily appreciate that other manufacturing techniques could be used. The main body 52 may be constructed of one monolithic piece of material such that the central region 100 and peripheral region 102 are integral with respect to each other. The main body 52 may be constructed of a polymer material.

Still referring to FIGS. 26-28B, the top surface 118 may have a round footprint, e.g., be of roughly circular shape, and may be convex, forming a dome shape (including the periphery 124). The top surface 118 may include slots 126. The slots 126 may be cutouts, apertures, holes, openings, or voids in various embodiments. The slots 126 may extend radially with respect to a center point 128 of the top surface 118 such that their respective first endpoints 130 surround a region including the center point 128 of the top surface 118 and their respective second endpoints 132 may each terminate a distance (e.g., the slots 126 may each terminate the same distance) from the periphery 124 of the top surface 118. In certain embodiments, the slots 126 may be disposed at regular angular increments and each be of equal length (though this need not be true to all embodiments).

Still referring to FIGS. 26-28B and as described above with reference to FIG. 23, the slots 126 could, in an alternative embodiment, be disposed such that they do not extend radially with respect to the center point 128. For example, the slots 126 may each extend at a common angle with respect to the radial direction. In such embodiments, slots 126 may be evenly spaced about the top surface 118 and may each be of the same length. In other embodiments, the slots 126 may not all extend at a common angle to the radial direction. At least one of the slots 126 (and perhaps all) may be disposed at a different angle to the radial direction.

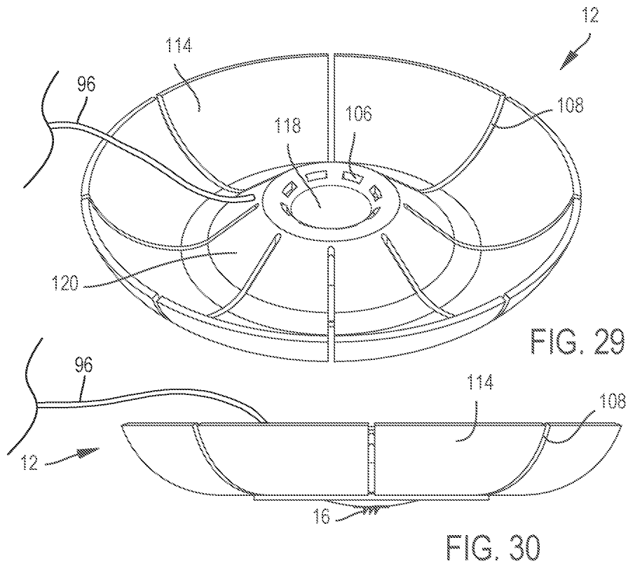

Referring now primarily to FIGS. 29-30, a number of views of a conceptual representation of a set 12 in a delivery state are shown. As described above (and also with reference to the embodiments of FIGS. 13-21), the set 12 may, upon downward pressure being applied to the top surface 118, transition from a storage state to a delivery state in which the main body 52 of the set 12 is substantially, or at least partially, inverted. A user may remove an adhesive liner 140 (see, e.g., FIG. 25) from the set 12 and apply the set 12 to the skin 14. The user may then press downward (i.e., toward the skin 14) on the top surface 118. This may cause the petal members 114 to spreadingly displace outward and curl upward (over at least a portion thereof), stretching the skin 14. The top surface 118 may invert, driving access member(s) 16 into the skin 14, and remain inverted when the set 12 attains the delivery state. The peripheral region 102 may also take on an inverted shape due to the curling of the petal members 114.

In various embodiments, certain regions of the main body 52 of the set 12 may remain static or may not invert. Thus, a main body 52 may include inverting regions and resilient regions. Though described as resilient regions, it is to be understood that some bending or deformation may still occur as pressure is applied. These regions may, however, appear generally similar or extend/project in the general same direction in both the storage and delivery state. As shown, the peripheral region 102 and top surface 118 may invert, but a portion of the central region 100 may resist deformation to this degree. The supporting structures 120 shown in other embodiments described herein (see, e.g., FIG. 20 or FIG. 26) may also be a resilient region. Thus, certain sets 12 may include a main body 52 with invertible regions which are separated from one another by a resilient region. The slots 126 may aid the set 12 to transition from the storage state to the delivery state with reduced pressure from above. The fenestrations 106 may also facilitate the transition.

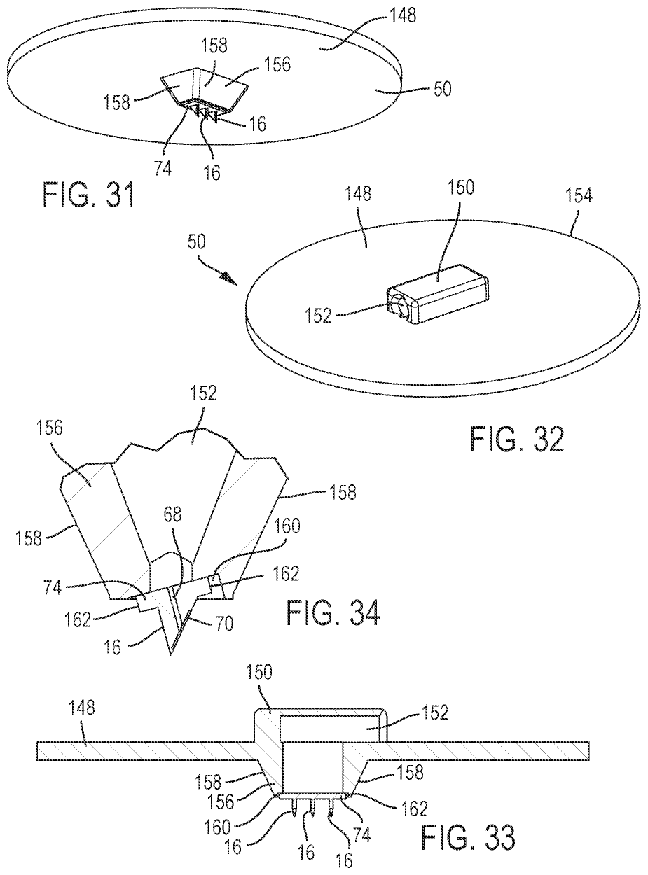

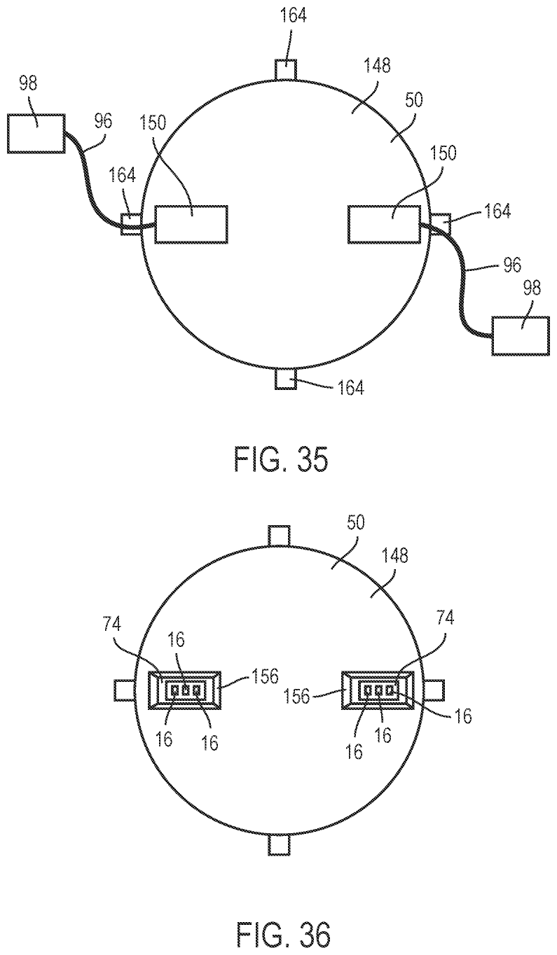

In some embodiments, as shown in FIGS. 31-33 (respectively a perspective view from the bottom, perspective view from the bottom, and cross-sectional view), an example body 50 for mounting of sharp bearing body 74 including access member(s) 16 is shown. Some example bodies 50 may be formed as a round (e.g. generally circular) body 148. The round body 148 may be substantially disk like or planar in various examples. A distal side of the body 50 may include a conduit receiver 150 into which a terminal end of a conduit 96 (see, e.g., FIG. 13) extending from a coupling 98 (see, e.g., FIG. 13) may be plumbed. The conduit receiver 150 may include a passage 152 which may be in communication with the flow lumens 68 of any access members 16 mounted to the body 50. The conduit 96 may be coupled into the passage 152 in any suitable manner (e.g. via adhesive). The body 50 may be manufactured by any technique known to those of skill including, e.g., injection molding. The distal face of the body 50 may couple to a portion of a main body 52 of a set 12. For example, a periphery 154 of the distal face may be coupled to an interior ridge 142 (see, e.g., FIG. 27) of the main body 52. In certain examples, one or more tabs 164 (see, e.g., FIGS. 35-36) extending from the peripheral edge of the body 50 may couple (e.g. snap) into slits in the main body 52 to couple or aid in coupling the body 50 to the main body 52.

Still referring to FIGS. 31-33, example bodies 50 may include at least one stage projection 156. A stage projection 156 may extend from a proximal side of a round body 148. Example stage projections 156 may extend proud of the proximal side by a height which may, in certain examples, be at least equal to the height of a microneedle (e.g. 600 microns) of the set 12. A stage projection 156 may generally extend from the round body 148 at a perpendicular angle. The side walls 158 of a stage projection 156 may be chamfered so as to extend in a non-perpendicular direction with respect to the proximal face of the round body 148. A stage projection 156 may include a pocket 160. The pocket 160 may be sized to fit and accept a sharp bearing body 74 with access member(s) 16 thereon. The sharp bearing body 74 may be mated into the pocket 160 by adhesive in the example shown in FIGS. 31-33. A stage projection 156 may act as a force concentrating protuberance extending from the rest of the body 50 which serves to ensure force applied to a set 12 is concentrated upon the access member(s) 16 aiding in insertion of the access member(s) 16 into the skin 14 (see, e.g., FIG. 1).

A sharp bearing body 74 may be coupled to any of a body 50 during a molding operation in certain examples. Where the sharp bearing body 74 is joined to a body 50 during molding, some material may be molded up the sidewalls 162 of the sharp bearing body 74 and over onto the face of the sharp bearing body 74 from which the access member(s) 16 project to capture the sharp bearing body 74. In alternative embodiments, the sidewalls 162 of the sharp bearing body 74 may be chamfered or at an angle which is not perpendicular to the face of the sharp bearing body 74 from which the access member(s) 16 extend. The footprint or cross-section of the sharp bearing body 74 may increase in area as distance from the sharp bearing face of the sharp bearing body 74 increases. Where the access member(s) 16 is/are silicon microneedles, a number of sets of access members 16 may typically be formed on a large wafer and sharp bearing bodies 74 including the desired number of access member(s) 16 may be diced out of the wafer. To form the chamfered sidewalls 162, the dicing saw may have angled faces such that dicing process creates the desired chamfer or angle on the sidewalls 162. In certain embodiments, sidewalls 162 which are between 30-60° (e.g. 45°) may be used. Where chamfered sidewalls 162 are present, material may be molded up only a portion of the sidewall 162 to couple the sharp bearing body 74 to a body 50. This may allow for a sharp bearing body 74 to be captured in a body 50 without material being molded over onto the sharp bearing face of the sharp bearing body 74 (though this could optionally be done). Thus no molded material may act as a stand-off on the sharp bearing face blocking the full height of any access member(s) 16 from penetrating into the skin 14 (see, e.g. FIG. 1).

Referring now also to FIG. 34, a cross-sectional view of a portion of a stage projection 156 is depicted. In some embodiments, the pocket 160 of the stage projection 156 may be in a non-parallel orientation with respect to the plane of the round body 148 (and the skin 14 when the set 12 is applied to a user). When a sharp bearing body 74 is mounted to the pocket 160, the orientation of the pocket 160 may ensure that the access member(s) 16 (e.g. microneedles) extend at a prescribed angle with respect to the round body 148. In the example embodiment, the pocket 160 may be oriented such that the access member(s) 16 extend at a 10-20° angle (e.g. 15°) with respect to a plane perpendicular to the round body 148. In other embodiments, the pocket 160 may be oriented such that the access member(s) 16 project at a 45° or 60° angle or some angle between 10-60°. Any suitable angle may be used. In alternative embodiments, the entire stage projection 156 may project at a desired angle from the round body 148. Thus, the access member(s) 16 may extend at a desired angle with respect to a plane perpendicular to the round body 148 when coupled to the pocket 160.

Referring now to FIGS. 35-36 top and bottom plan views of an example embodiment of a body 50 are respectively shown. As mentioned above, in certain examples, a body 50 may include tabs 164 which may couple or aid in coupling the body 50 to a main body 52 of a set 12. In the example shown, a round body 148 may include a number of peripherally disposed tab projections 164. The tab projections 164 may be symmetrically disposed about the round body 148 and may be spaced at regular angular intervals. In alternative embodiments, the tab projections 164 may be asymmetrically disposed about the base or disposed at irregular angular intervals. The tab projections 164 may engage with receiving slits disposed in a main body 52 of a set 12. Thus, the tab projections 164 may be used to couple the body 50 into place in a set 12. Asymmetric or irregularly spaced tab projections 164 may allow for the body 50 to be coupled to a main body 52 in a prescribed orientation which may be desirable in some examples

Still referring got FIGS. 35-36, the example body 50 includes a plurality of conduit receivers 150 which may each accept a conduit 96 in fluid communication with a respective coupling 98. In alternative embodiments, the conduit receivers 150 may be omitted and couplings 98 may be provided directly on the body 50. For example luer fittings may be coupled to or molded as part of the body 50. In certain embodiments, couplings on the body 50 may be the couplings 98 described in relation to FIGS. 41-46.

The body 50 may include a plurality of stage projections 156. A stage projection 156 to which a sharp bearing body 74 may be coupled may be associated with each of the conduit receivers 150 included on the body 50 (or alternatively couplings 98 on the body 50). Though two stage projections 156 and conduit receivers 150 are shown, a greater number may be included in alternative examples.

The stage projections 156 may, for example, be any of those described herein such as those shown in FIGS. 31-34. The conduit 96 coupled into each of the conduit receivers 150 may be in fluid communication with the flow lumens 68 (see, e.g., FIG. 34) of access member(s) 16 coupled to a respective stage projection 156. Alternatively, where couplings 98 are included on the body 50 in place of the conduit receivers, the couplings 98 may be in fluid communication with the flow lumens 68. The flow path from a first of the conduit receivers 150 (or first coupling 98) to access member(s) 16 coupled to a respective first stage projection 156 may be fluidically isolated from the flow path from any other conduit receiver 150 (or couplings 98) to access member(s) 16 coupled to the respective stage projection 156. Each of the couplings 98 may couple to a connector 26, 44 from an infusion device 18 (see, e.g., FIG. 2) of the system 10 (see, e.g., FIG. 2).

A first agent may be administered through access member(s) 16 coupled to a first of the stage projections 156 and different agent(s) may be administered through access member(s) 16 coupled to other stage projections 156. For example, agents with counteracting effects may be administered. A first regulatory hormone and a second regulatory hormone that counters the first regulatory hormone could be administered. In some examples, insulin and glucagon may be delivered. Alternatively, the same agent may be administered through the access member(s) 16 coupled to each of the stage projections 156. Alternatively, different types of the same agent may be administered through access member(s) 16 coupled to each of the stage projections 156.

The access member(s) 16 coupled to each of the stage projections 156 may be identical or substantially identical. In certain examples, one stage projection 156 may have a greater or lesser number of access member(s) 16 coupled thereto than another of the access member(s) 16. In certain examples, the access member(s) 16 coupled to one of the stage projections 156 may be longer or shorter than the access member(s) 16 coupled to another of the stage projections 156. Stage projections 156 with access member(s) 16 of any number of heights may be included. Using insulin as an example, insulin may be delivered through shorter access member(s) 16 (e.g. to an intradermal destination) if a fast response is needed or desired and may be delivered through longer access member(s) 16 (e.g. to subcutaneous destination) in other scenarios. Routing of agent to longer or shorter access member(s) 16 (or access member(s) 16 of different heights at the same time) may be governed by the controller 20 of an infusion device 18. The access member(s) 16 selected for delivery could be determined by the controller 20 based at least in part on how quickly it is desired for the delivered agent to act.

Where a plurality of stage projections 156 are provided, the stage projections 156 may be arranged in a row on the body 50. The stage projections 156 may each have the same orientation as shown in FIG. 36. The stage projections 156 may be disposed substantially along a midplane of the body 50 in some examples. The access member(s) 16 coupled to the stage projections 156 may each have the same orientation. In certain examples where microneedles are used, the back facing edge 66 (see, e.g., FIG. 5) of each of the microneedles may all be disposed in substantially the same plane. The stage projections 156 may be disposed on opposing sides of the proximal face of the body 50. Alternatively, the stages projections 156 may each be centrally disposed on the body 50. In certain embodiments, only a single stage projection 156 may be included and multiple sharp bearing bodies 74 may be coupled thereto. Flow paths from the conduit receivers 150 to each respective sharp bearing body 74 may be isolated from one another by a wall or partition within the interior of the stage projection 156.

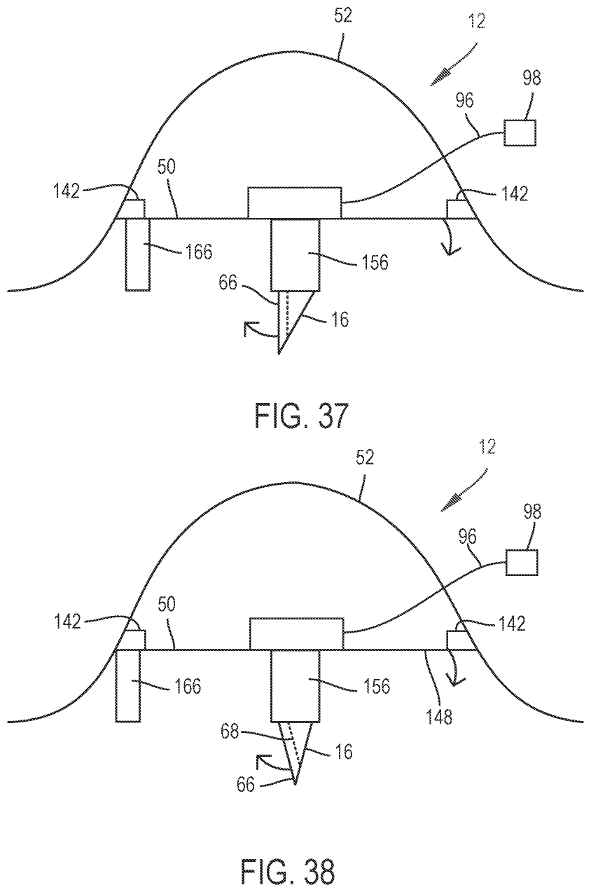

Referring now to FIGS. 37-38, various of the sets 12 described herein may include a body 50 with at least one rocker member 166. When such a set 12 is applied to a user and transitioned to a delivery state, skin 14 (see, e.g. FIG. 1) may be rendered taught due to spreading displacement of portions of the set 12 and access member(s) 16 of the set 12 may displace into the stretched skin 14. Movement of the access member(s) 16 may generally be in a first direction which is substantially perpendicular to the surface of the skin 14 and the access member(s) 16 may generally puncture downwardly into the skin 14. The at least one rocker member 166 may cause the body 50 to tilt or rock as a consequence of the set 12 being transitioned to a delivery state. The at least one rocker member 166 may cause the access member(s) 16 to displace slightly in a second direction substantially opposite the first direction when pressure is relieved from the set 12. Tilting as well as displacement in the second direction may occur.

In some embodiments, portions of the set 12 may also deform or adjust in response to the rocking of the body 50 in order to accommodate the rocking of the body 50. The tilting of the body 50 may cause the access member(s) 16 to displace in a non-straight path. For example the access member(s) 16 may rotate or swing along an arcuate path during at least a portion of the transition of a set 12 to the delivery state. In example embodiments, the tilting may occur automatically as a consequence of the transition of a set 12 to a delivery state. No linkages or interactions with guide elements may be needed in order to achieve the tilting. Example bodies 50 may tilt together as a single unit due to the presence of the one or more rocker member 166. Such tilting of a body 50 may lower the pressure at which injection may begin to occur and/or increase delivery flow rate in certain set 12 embodiments. Additionally, the inclusion of one or more rocker member 166 may impact characteristics of bleb formation during delivery. Tilting may also help to facilitate delivery where access member(s) 16 are initially advanced into skin 14 at an angle substantially perpendicular to the skin 14.

Still referring to FIGS. 37-38, a rocker member 166 may be a protrusion which extends from a proximal face of a body 50. In various examples, a rocker member 166 may be disposed at or inward of the peripheral edge of the body 50. A rocker member 166 may have a height which is approximately the height of a stage projection 156 (see, e.g., FIGS. 31-36). Shorter and taller rocker members 166 are also possible.

When sets 12 including at least one rocker member 166 are transitioned to a delivery state, the rocker member(s) 166 may come into contact with the user and impede further displacement of the portion of the body 50 including the rocker member(s) 166. The opposing side may be free of any rocker members 166 and the body 50 may tilt or rock to accommodate continued displacement of the opposing side toward the user. In certain examples, the access member(s) 16 (e.g. microneedles) may tilt 3-5° (e.g. 4°) with respect to their initial orientation. In other examples, the access member(s) 16 may tilt lesser or greater amounts. Height of a rocker member 166 may alter the point at which the access member(s) 16 begin to rotate or swing during the transition of the set 12 to the delivery state. Rocker members 166 even with the height of a stage projection 156 may, for example, tend to initiate tilting after the access member(s) 16 have punctured the skin 14 (see, e.g., FIG. 1).

In certain examples, the access member(s) 16 may be microneedles such as any of those described herein. Where the access member(s) 16 is/are microneedle(s), the rocker member(s) 166 may be disposed on a side of the body closest the back facing edge 66 (see, e.g., FIG. 5) of the microneedle(s). The rocker member(s) 166 may be positioned such that back facing edge 66 of the microneedle(s) is the portion of the microneedle(s) most proximal the rocker member(s) 166. As rocking of the body 50 transpires, the displacement path followed by the microneedle(s) may be such that the back facing edge(s) 66 may be driven through the skin. The beveled surfaces leading to the back facing edge 66 may facilitate cutting of the skin 14 (see, e.g., FIG. 1) as the microneedle(s) are displaced. Thus, the back facing edge 66 may be a cutting edge. Additionally, this may cause a face of each microneedle in which an outlet of the lumen 68 of that microneedle is disposed to be displaced away from skin contacted during the initial puncture. For example, the lumen(s) 68 of any microneedles may be displaced away from skin 14 contacted by the sloped face(s) 72 during the initial puncture where a microneedle such as that shown in FIG. 5 is utilized. Such displacement of the microneedle(s) may aid in ensuring fluid may easily flow out of the lumen(s) 68 and into the skin 14 as delivery occurs. The above described displacement may also create a small receiving volume in the skin 14 into which fluid may be delivered from the lumen(s) 68. When pressure applied to the set 12 to transition the set 12 to the delivery state is relieved, the access member(s) 16 may displace slightly in a direction away from the patient. This may create a small receiving volume in the skin 14 and displace the lumen(s) 68 away from skin 14 contacted during initial puncture. The rocker member 166 may help to encourage this.

In some examples (see, e.g., FIGS. 34 & FIG. 38), access member(s) 16 may be mounted to a stage projection 156 having a mounting area (e.g. a pocket 160) which is non-parallel with respect to a round body 148 of the body 50. In such examples, the access member(s) 16 may extend from the stage projection 156 at a prescribed angle (e.g. 15°) with respect to a plane normal to the round body 148. The round body 148 and skin 14 (see, e.g., FIG. 1) may be generally parallel when various example sets 12 are first applied to a user. Access member(s) 16 may thus be angled with respect to a plane normal to the skin 14. As the body 50 tilts, the access member(s) 16 may be displaced to a position in which they are closer (e.g. 3-5°) to a normal orientation with respect to the skin 14. Depending on the mounting angle of the access member(s) 16, the access member(s) 16 may be brought to or nearly to a normal orientation with respect to the skin 14 as the body 50 tilts. In other embodiments, the access member(s) 16 may be 10° or more (e.g. 11-12°) away from a normal orientation.

In some examples, the stage projection 156 may be disposed in the position of the rocker member 166 shown in FIGS. 37-38. In such embodiments, the rocker member 166 may be omitted or may be present as lateral extensions on either side of a stage projection 156. No stage projection 156 may be present in the center of the body 50 (though a stage projection 156 could also be centrally disposed on the body 50 in particular examples). As the set 12 transitions to the delivery state, the access member(s) 16 may puncture the skin 14 and the stage projection 156 may block further displacement of that side of the body 50 toward the skin 14. The opposing side of the body 50 would be free to continue displacement toward the skin 14. As the opposing side continues displacing toward the skin 14 a rocking or tilting which causes movement of the access member(s) 16 in the manner described above may occur.

When pressure is removed from the set 12, one or more portion of the set 12 may at least partially restore from its distorted state. Thus, the set 12 may have at least one region which elastically deforms as a set 12 is transitioned to a delivery state. The at least one region which elastically deforms may be distorted from an initial state, to an intermediate state, and then elastically restore at least partially from the intermediate state during the course of the transition to the delivery state. The intermediate state may be a state during the transition in which the region is maximally distorted. The region may restore from this state back towards the initial state. The petal members 114 may, for example, at least partially restore from their maximally distorted state. As the petal members 114 of the set 12 are adhered to the skin 14 via the adhesive 54 of the set 12, the skin 14 may be pulled away from the underlying anatomy as the petal members 114 restore. This may relieve some pressure on the injection site. This decreased compression at the injection site may allow fluid to be more readily be transferred from the access member(s) 16 into the delivery destination. Additionally, depending on the orientation of the access member(s) 16, the access member(s) 16 may tug the skin 14 into which they have punctured upward away from underlying anatomy as the petal members 114 restore. Again, this may help to facilitate delivery as the compactedness of the anatomy at the delivery destination may be reduced. The shape of the petal members 114 and material used to construct the set 12 may be selected to help encourage this at least partial restoration or recoil of the petal members 114 when pressure is removed. Molding the petal member 114 in the storage state may also bestow a tendency from the petal members 114 to restore toward the storage state when pressure is relieved from the set 12 during use. Petal members 114 which restore towards their initial state during transition of a set 12 to a delivery state may be included in any set 12 embodiment shown or described herein.

Petal members 114 may be relatively devoid of curvature. For example, petal members 114 be substantially flat and/or extend from the rest of a main body 52 at an angle or angles thereto. This may assist in making the force required to cause deflection in the petal members 114 relatively low as pressure is applied to the set 12. In turn, this may help to assist in generating spreading displacement of the petal members 114 and help ensure puncture of the skin with the access member(s) 16 prior to deformation of a top surface 118 of an example main body 52.

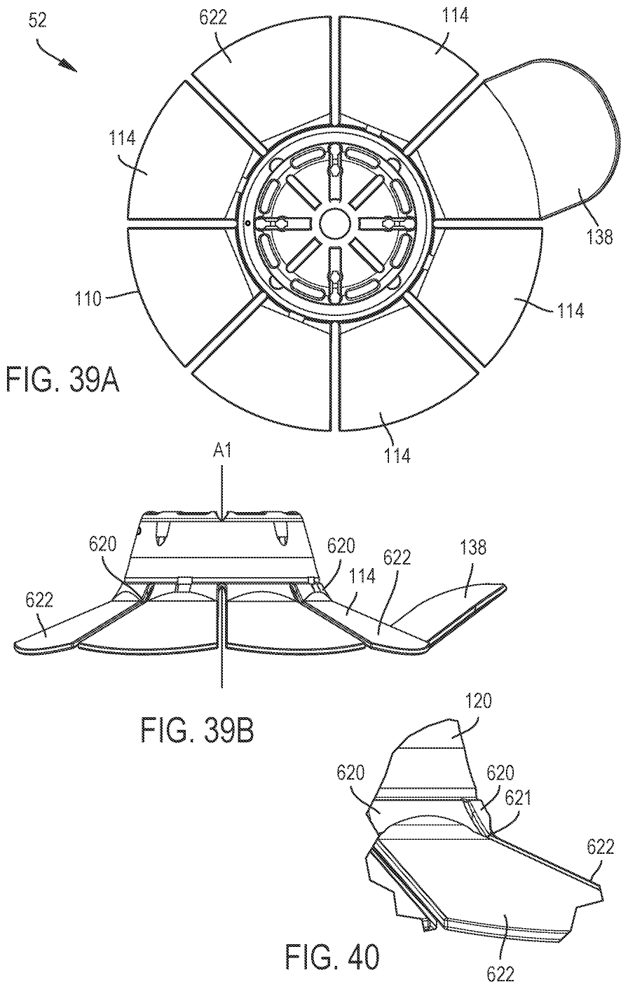

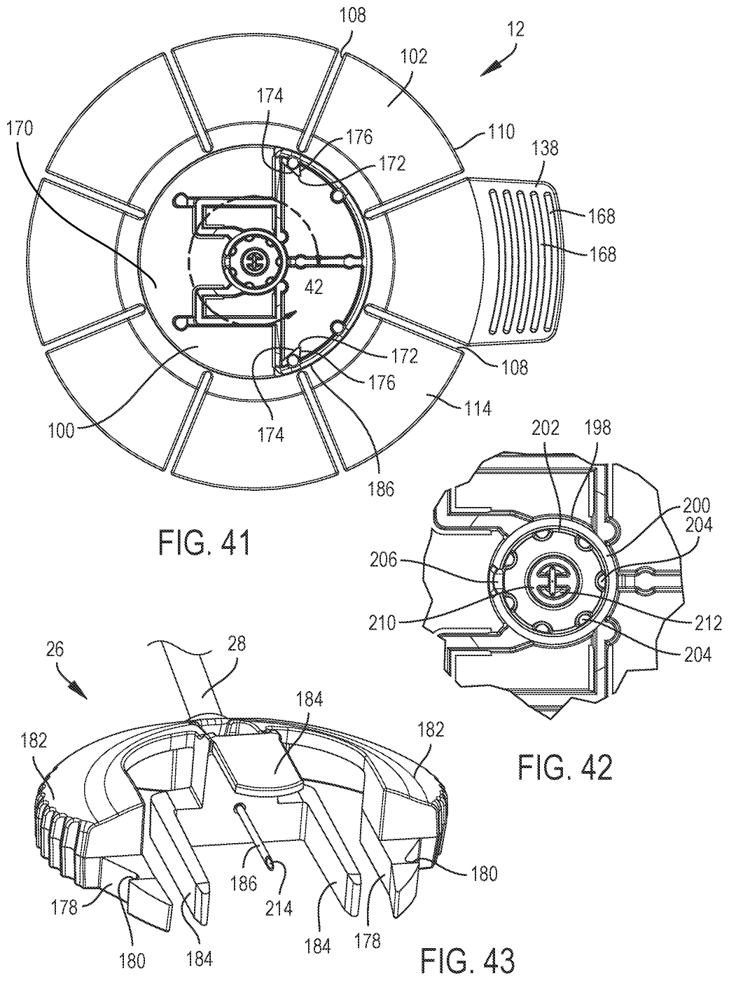

As shown in FIGS. 39A-39B, the petal members 114 of example main bodies 20 may each include first regions 620 adjacent the supporting structure 120 and second regions 622 which form the more peripheral portions of the petal members 114. As shown, the first regions 620 may be arced roughly similar to that of the adjacent portion of the supporting structure 120. The second regions 622 may be oriented at a constant angle to the center axis A1 of the main body 20. The second regions 622 may form the majority of the petal members 114. In some examples, the petal members 114 may have a curved region or surface, while being predominantly flat. In various embodiments, a small curved transition 621 between the first and second regions 620, 622 of the petal members 114 may be included (see, e.g., FIG. 40).

A living hinge may be formed at the transition between the first and second regions 620, 622. As pressure is applied to the set 12, the living hinge may allow the second regions 622 of the petal members 114 to displace relative to the first regions 620. The first regions 620 may distort to a lesser degree than the second regions 622 throughout the transition of the set 12 to the delivery state. In some examples, the first regions 620 may resist substantial deformation and remain generally undistorted throughout the transition. Thus, the first regions 620 may behave as stops which may help to limit spreading displacement of the petal members 14 after a desired amount of spreading displacement has been achieved. Curved transitions 621 may be included to assist in encouraging the petal members 114 to at least partially restore once pressure on the set 12 has been relieved. In examples including petal members 114 such as those in FIGS. 70A-70B, it may be preferred that, in the storage state, the base of any stage projection 156 be substantially even with the end of the second region 622 of the petal members 114 most proximal the supporting structure 120.

Referring now to FIGS. 41-42, an example embodiment of a set 12 is depicted. The example set 12 includes a central region 100 and a peripheral region 102. The peripheral region 102 includes a plurality of slots 108 which divide the peripheral region 102 into a plurality of petal members 114. One of the petal members 114 includes a pull tab 138. The example pull tab 138 extends in a direction which is substantially within a plane including the peripheral edge 110 of the peripheral region 102. When the set 12 is worn by a user, the pull tab 138 may generally lay flat against the skin 14 to minimize potential for the pull tab 138 to catch on clothing or other items. The pull tab 138 may include one or more raised body 168 or gripping pattern to facilitate grasping of the pull tab 138 by a user. In the example the raised bodies 168 included are ridges.

The central region 100 in the example embodiment is shown including a base surface 170 from which a number of features extend. The base surface 170 is depicted as a plateau like surface in the example and is substantially planar. The base surface 170 may be disposed above the peripheral region 102 or even with a highest portion of the peripheral region 102. The central region 100 may include a coupling 98 to which a connector 26 from an infusion device 18 may be coupled. In various embodiments, a fitting (e.g. luer lock) may be coupled with the central region 100 and serve as the coupling 98. In some embodiments, the fitting may be molded integrally with the central region 100.

Referring now also to FIG. 43, a central region 100 may, for example, include connector receivers 172. The connector receivers 172 may be connector engaging projections extending from the base surface 170. The connector receivers 172 may include a ramped face 174 and a ledge face 176. As latch bodies 178 of an example connector 26 are driven toward the connector receivers 172, they may abut the ramped faces 174 of the connector receivers 172. In various embodiments, the latch bodies 178 may be cantilevered from the rest of the connector 26. Further displacement of the connector 26 may result in deflection of the latch bodies 178 as the latch bodies 178 are driven along the ramped faces 174. A terminal end of the latch bodies 178 may include a face ramped in cooperating manner to help facilitate deflection. The latch bodies 178 may include a catch 180. As the catches 180 clear the respective ramped faces 174, the latch bodies 178 may be free to restore from their deflected state. As the latch bodies 178 restore from the deflected state, the catches 180 may displace into engagement with the ledge face 176 of the connector receivers 170 to retain the connector 26 in place on the set 12. Thus, the connector receives 172 may form the coupling 98 on the set 12. In some embodiments, the catches 180 may include faces that are substantially parallel to the ledge faces 176 of the connector receivers 172 when engaged with the ledge faces 176. In alternative embodiments, the catches 180 may include faces which are non-parallel with the ledge faces 176 when connector 26 is coupled to the set 12. This may facilitate breakaway for the connector 26 from the set 12 in the event that tubing 28 leading from the connector 26 to an infusion device 18 is snagged as a user moves about. The angle of the faces of the catches 180 may be adjusted to alter the ease with which the connector 26 may be disassociated from the set 12 in such a scenario.

The connector 26 may be disengaged from the set 12 manually as desired. This may be done, for example, if a user desires to shower or swim and the infusion device 18 is not suited for such exposure to water. As shown, the latch bodies 178 extend from outer arms 182 of the connector 26. The outer arms 182 may themselves be cantilevered from a central region of the connector 26. The outer arms 182 may be squeezed together to displace the latch bodies 178 out of engagement with the connector receivers 170 to allow the connector 26 to be removed. In the example embodiment, as the outer arms 182 are deflected toward one another, the catches 180 may be displaced out of contact with the ledge faces 176 and the latch bodies 178 may then be free to be withdrawn away from the connector receivers 172.

Sharp flanking projections 184 may also be present on the connector 26. These flanking projections 184 may extend substantially parallel to a sharp 186 included on the connector 26 and may present an obstacle which helps block accidental contact between the sharp 186 and the user. A shielding wall 188 may be provided on the central region 100 and may help to block fingers or objects from inadvertently dislodging the latch bodies 178 out of engagement with the connector receivers 172.

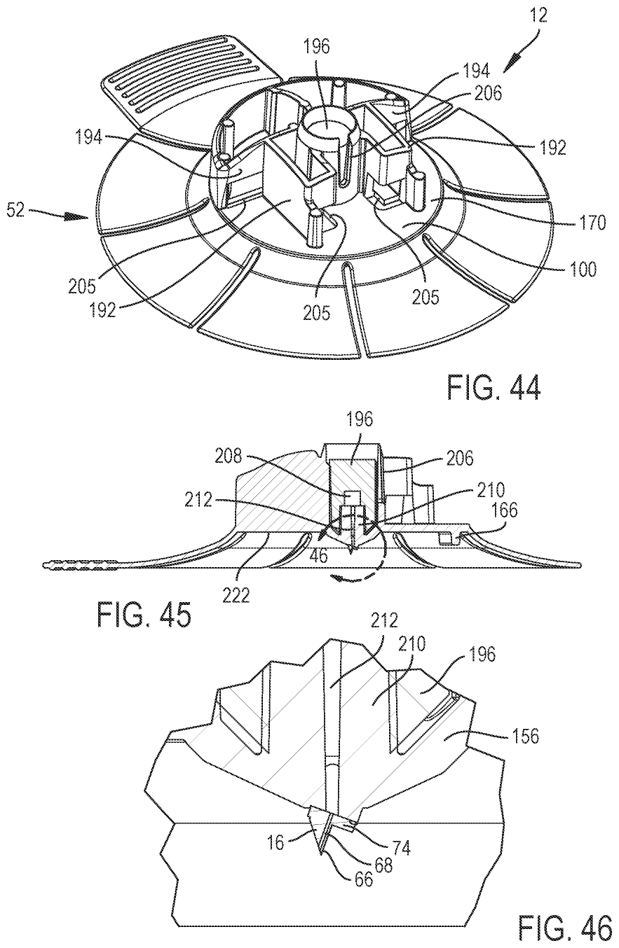

Referring now also to FIG. 44, the central region 100 of the set 12 may include a number of guides. The guides may facilitate coupling of a connector 26 to the set 12. As shown, a set of guide walls 192 project from the base surface 170 of the central region 100. The guide walls 192 may be generally perpendicular to the base surface 170 in various embodiments. As a connector 26 is coupled to the set 12, at least one of the latch bodies 178 or flanking projections may ride along one of the guide walls 192 to aid in aligning the connector 26 along a desired displacement path as it is coupled to the set 12. This may ensure that a sharp 186 of the connector 26 punctures a septum 196 of the set 12 in along a desired puncture axis. Guides may also may help make it easier to couple a connector 26 to a set 12 for individuals with low dexterity or impaired vision. In some examples, guides may help direct the connector 26 into an appropriate coupling pathway such that a user may couple the connector 26 to the set 12 without needing to look at the set 12.

The set 12 may also include constraining walls 194 which may, for example, prevent movement in certain directions of portions of the connector 26. For example, a bridge of material extends from the shielding wall 188 to the guide walls 192. This may prevent displacement of the latch bodies 178 in a direction perpendicular to the base surface 170 when the connector 26 is coupled to the set 12. This may inhibit the latch bodies 178 from being lifted over the connector receivers 172 to disengage the connector 26 from the set 12. In the example embodiment the guides and constraining walls 194 are arranged so as to be about even with the top surface of the connector 26 when the connector 26 is coupled to the set 12. This may help to limit potential for the set 12 or connector 26 to snag on clothing or other items when in use.

Additionally as shown, a set 12 may include a septum bay 198 (best shown in FIG. 42). The septum bay 198 may be generally centrally disposed on the central region 100 of the set 12. The septum bay 198 may be defined by a wall 200 raised from the base surface 170. The wall 200 may surround an interior void into which a septum 196 (shown in FIG. 44) may be deposited during assembly. The interior surface 202 of the wall 200 may include a number of ribs 204 which extend radially inward toward an axis of the septum bay 198. The ribs 204 may help to locate and compress the septum 196 within the septum bay 198.

The top of the septum 196 may be at least partially covered once the septum 196 has been installed in the septum bay 198. In certain examples, the wall 200 may include an extended region 208 most distal to the base surface 170. The extended region 208 may be swaged over after the septum 196 is installed to help retain the septum 196 in place within the septum bay 198. In alternative embodiments, a plug, cap, or cover may be coupled to the top of the wall 200 to cover the exposed face of the septum 196. Such a plug, cap, or cover may be coupled in any suitable manner (e.g. snap fit, threaded coupling, welding such as sonic welding, etc.).

Referring now to FIG. 45 in addition to FIGS. 41-44, the septum bay 198 may include a post 210. The post 210 may be substantially centrally located within the septum bay 198. The post 210 may include a passage 212 extending therethrough which is in fluid communication with the access members 16. The passage 212 may have a cross-section generally in the shape of the Latin character “I”. This may help to minimize dead volume of the passage 212 while maintaining substantially uniform wall thickness for the post 210. As best shown in FIG. 45, the septum 196 may include a septum recess 208 which may seat at least partially on the post 210 and form a fluid tight seal against the post 210. The post 210 may aid in centering the septum 196 in the septum bay 198 during assembly. A portion of the septum recess 208 may be disposed adjacent the post 210 when the septum 196 is installed in the bay 198. This portion may provide a fluid introduction volume into which fluid from a connector 26 may be delivered.

The wall 200 of the septum bay 198 may include a port 206 such as a notch or fenestration (not shown). When a connector 26 is coupled to the set 12, the sharp 186 of the connector 26 may be displaced along a displacement path which extends through the port 206 and through a portion of the septum 196. An outlet 214 of the sharp 186 may be disposed within the fluid introduction volume formed by the recess 208 of the septum 196 when a connector 26 is coupled to the set 12. As fluid pump by an infusion device 18 (see, e.g. FIG. 1) is expelled through the outlet 214 of the sharp 186, the fluid may travel though the passage 212 and flow lumen 68 of any access members 16 of the set 12 to reach a delivery destination in a patient. The septum 196 may be formed of a material which may self-seal upon puncture and removal of a sharp 186. In certain examples, the septum 196 may be a silicone material.

It may be preferable to include a notch which extends from the top of the wall 200 toward the base surface 170 in various examples as it may facilitate molding of the set 12 without the need for side actions. The example set 12, for example could be molded using bypass shutoffs and no side actions. Various gaps or apertures 205 in the central region 100 may be included to facilitate molding.

Referring now primarily to FIGS. 45-46, the proximal side 222 (side facing the skin 14 when the set 12 is worn) of the central region 100 may include at least one stage projection 156. A sharp bearing body 74 including at least one access member 16 may be coupled to each stage projection 156 (e.g. via adhesive or may be joined to the stage projection 156 during injection molding). The sharp bearing body 74 may be disposed such that the access member(s) 16 extend at a desired angle with respect to a plane perpendicular to the proximal side 222 of the central region 100. In certain examples, the angle may be between 45-5 degrees (e.g., 30° or 15°). In alternative embodiments, the access member(s) 16 may project substantially perpendicular to the proximal side 222 of the central region 100. In the example embodiments, the access member(s) 16 are depicted as microneedles.

A rocker member 166 is also included near the periphery of the proximal side 222 of the central region 100. As the set 12 is transitioned to the delivery state (set 12 shown in storage state in FIGS. 45-46), the access member(s) 16 may puncture into the skin 14 (see, e.g., FIG. 1) and the central region 100 may begin to tilt once the rocker member 166 contacts the skin 14. As described elsewhere herein, the tilting motion may cause the access member(s) 16 to displace along a curved path within the skin 14. This may aid in facilitating delivery and help lower the pressure at which delivery into the skin 14 may begin to occur.

As shown in FIGS. 41-46, the example set 12 does not include a body 50. Instead, a stage projection 156 is formed on a proximal side 222 of the central region 100. Any of the sets 12 described herein as including a body 50 may alternatively be constructed such that features of the body 50 are instead included in the central region 100 of the main body 52. For example, the bodies 50 shown and described in relation to FIGS. 31-38 may be included as at least part of a central region 100 of a set 12 and may be integrally formed with a peripheral region 102 of a main body 52. This may help to limit part count and simply assembly of a set 12. That said, various sets 12 described herein may be modified such that features in FIGS. 41-46 which are described as part of the central region 100 may instead be included on a separate body 50 which may couple to a main body 52 of the set 12.

Referring now to FIGS. 47-49, an exploded view of an example embodiment of an analyte sensor 30 and transmitter 32 is shown. The transmitter 32 may include a housing 290. A power source 294 (e.g. coin cell), sensor circuitry 296, at least one transmitter/transceiver 298, and a memory 300 may be included within the housing 290. The transmitter 32 may removably couple to the analyte sensor 30 in any suitable manner. The transmitter 32 may thread, clip, snap, adhere, etc. to the analyte sensor 30, though any suitable manner of coupling may be used. When the transmitter 32 is coupled to the analyte sensor 30, the transmitter 32 may establish electrical communication with conductive traces 292 leading to sensor electrodes 302A, 302B. In other embodiments, the transmitter 32 may be provided coupled to the analyte sensor 30 and may not be removable.

As shown, the analyte sensor 30 may include a main body 352. The main body 352 of the analyte sensor 30 may have a round (e.g. circular) foot print and may include a central region 400 and a peripheral region 402. The central region 400 may be a raised region of the main body 352 and the peripheral region 402 may surround the central region 400. The main body 352 may be injection molded and the raised central region 400 and peripheral region 402 may be formed in the molding operation. The central region 400 may be substantially planar in some examples and may include a base surface 470. Conductive traces 292 communicating with the electrodes 302A, B may extend to the base surface 470 in various embodiments. The central region 400 may include one or more coupling interface which cooperates with a transmitter 32 to couple the transmitter 32 and analyte sensor 30 to one another during use. The main body 352 may include a number of slots 408. The slots 408 may extend from a peripheral edge 410 of the main body 352 toward a center or midpoint of the main body 352. In the example embodiment, the slots 408 extend in a radial direction. The slots 408 may extend through the entirety of the peripheral region 402. The main body 352 may thus include a central region 400 which is circumscribed by a number of petal members 414 which are spaced apart via the slots 408.

Referring now primarily to FIG. 48, a plan view of the proximal face 356 of an example main body 352 is depicted. Adhesive may be included on at least a portion of the proximal face 356. Various adhesive bearing members 54 which may be coupled to a proximal face 356 of a main body 352 of an analyte sensor 30 are shown and described in relation to FIGS. 55-57. The adhesive may be a skin compatible adhesive and may serve to couple an analyte sensor 30 to a skin 14 surface at a sensing site. Adhesive may be included on the peripheral region 402 of the main body 352 as well as a portion of a proximal face 422 of the central region 400.

The example analyte sensor 30 is depicted in a storage state in FIGS. 47-49. When the analyte sensor 30 is applied to a patient, the analyte sensor 30 may be placed on the skin 14 in the storage state. The adhesive on the proximal face 356 of the main body 352 may anchor the analyte sensor 30 to the skin 14. The main body 352 may be a deformable body which may transition from the storage state to a deployed state. In certain examples, this transition may be reversible, though in other embodiments the transition may result in a permanent change in the main body 352 and/or another part of the analyte sensor 30. For example, once transitioned to the delivery state, at least a portion of the main body 352 may plastically deform such that it is permanently distorted and may not be returned to the storage state. Destruction of a portion of the main body 352 or a portion of an analyte sensor may be required to remove the analyte sensor 30 from a patient rendering the analyte sensor 30 inoperative. As transition to a deployed state occurs, at least two adhesive bearing portions of the main body 352 may be spreadingly displaced with respect to one another so as to stretch or spread a skin surface anchored to the main body 352 via the adhesive. This may be desirable as the skin 14 may be rendered taught facilitating piercing of the skin 14 by the electrodes 302A, B as the main body 352 transitions to the deployed state. The adhesive present on an adhesive member 54 (see, e.g., FIGS. 55-57) may aid in holding the analyte sensor 30 in the deployed state during use. No inserter assembly may be needed to apply the analyte sensor 30.

The analyte sensor 30 may be applied to the skin 14 and a user may press down on the main body 352 to transition the analyte sensor 30 to the deployed state. This may avoid the need for a needle stick each time an analyte sensor 30 is applied. This may make use of an analyte sensor 30 more attractive, particular for certain patient populations (e.g. those with juvenile diabetes). Moreover, omission of an inserter may aid in defraying financial considerations of a patient which may dissuade some patients from opting to use an analyte sensor 30.

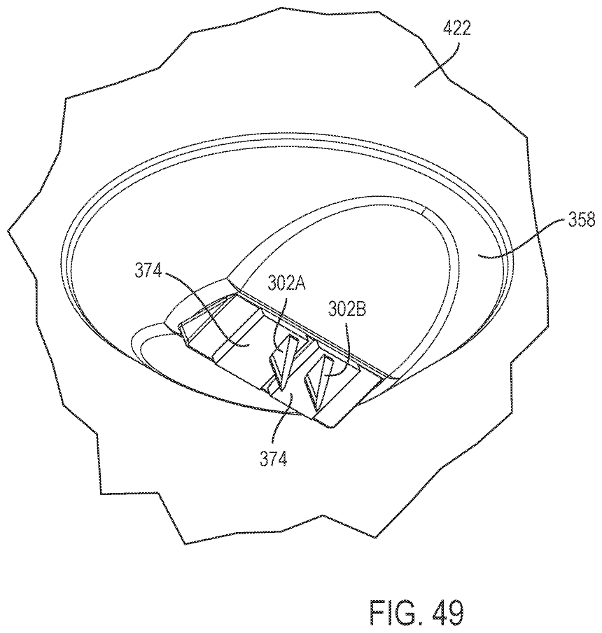

Referring now primarily to FIG. 49, a perspective close up view of a portion of the proximal face 422 of the central region 400 of the example analyte sensor 30 shown in FIGS. 47-48 is shown. As shown, the electrodes 302A, 302B may be a pair of micropenetrators. The micropenetrators be any of the microneedles described herein, but as shown may be devoid of any lumens extending therethrough. The micropenetrators may, for example, be structured as described in FIG. 5, but be lumen free. Each of the micropenetrators may be constructed of etched silicon. Each of the micropenetrators may be disposed on its own sharp bearing body 374 such that they are out of direct electrical communication with one another. One of the electrodes 302A, B may serve as a counter/reference electrode while the other electrode 302A, B may serve as a sensing electrode. The electrodes 302A, B may be mounted to a stage projection 358 which protrudes off of a proximal face 422 of the central region 400. The stage projection 358 may be any of the stage projections 156 described herein.

In alternative embodiments, the micropenetrators themselves may not be used as the electrodes 302A, B. In such embodiments, the micropenetrators may be at least partially covered with an insulative material. A conductive trace 292 may be provided over the insulative material on each micropenetrator and may extend along the sharp bearing body 374 to the side of the sharp bearing body 374 opposite the micropenetrator(s). The transmitter 32 may establish electrical communication with the conductive traces 292 when coupled to the analyte sensor 30. In such embodiments, the counter/reference electrode and the sensing electrode may be present on the same sharp bearing body 374 so long as the respective conductive traces 292 are kept isolated from one another.

The analyte sensor 30 may be a shallow analyte level sensor. For example, the analyte sensor 30 may be an intradermal analyte level sensor. The analyte sensor 30 may be a continuous analyte sensor 30 (e.g. continuous glucose monitor) which may output analyte level data on a predefined schedule (e.g. every 1-5 minutes). Such an analyte sensor 30 may be less invasive, less painful to use, more responsive, less prone to irritation or patient reaction among other potential benefits.

The electrodes 302A, B may have a height appropriate for puncture into communication with the shallow destination. In some embodiments, the electrodes 302A, B may have a height between 500-1000 microns though shorter or taller electrodes 302A, B may be used in alternative embodiments. As the analyte sensor 30 may sense analyte levels at a shallow destination (e.g. intradermal), the sensor 30 may collect readings of blood analyte levels which are less time delayed than subcutaneous analyte level sensors. The intradermal layer for example, is highly vascularized and thus analyte levels in the intradermal space should more rapidly reflect changes to levels in blood analyte levels. As sensor 30 readings are received by the controller 20, the controller 20 may initiate, suspend, or adjust agent administration commands used to orchestrate deliveries from the infusion device 18 based at least in part on the readings. More rapid sensing of analyte level changes may allow for a controller 20 of an infusion device 18 to react more quickly to changes in blood analyte levels. Additionally, it may allow for a controller 20 of an infusion device 18 to make agent administration determinations which more accurate reflect current needs of the patient. Thus, such an analyte sensor 30 may allow tighter control of blood analyte levels with an infusion device 18.

Any suitable analyte sensing chemistry or arrangement from any manufacturer may be incorporated on the sensing electrode. A glucose sensing arrangement may be used. For example, a glucose oxidase chemistry could be used to sense glucose levels in body fluid. It shall be noted that the analyte sensor 30 is not limited to using any particular sensing chemistry or arrangement.

In certain example embodiments, an analyte sensor 30 may include multiple stage projections 358 each associated with a respective set of electrodes 302A, B. Alternatively, multiple sets of isolated electrodes 302A, B may be coupled to a single stage projection 358. Thus a plurality of analyte sensors 30 may be associated with a single main body 352. In various examples, any number of stage projections 358 may be disposed about the proximal face 422 of a central region of a main body 352. In some examples, stage projections 358 on such an analyte sensor 30 may be arranged similarly to the stage projections 156 depicted in relation to FIGS. 35-36. Sets of electrodes 302A, B included in the analyte sensor 30 may use the same analyte sensing chemistry or arrangement and sense the same analyte in the body fluid. In other embodiments, at least one set of electrodes 302A, B may use a different analyte sensing chemistry or arrangement. Where different chemistries are used, the electrodes 302A, B may be sensing the same analyte as other sets of electrodes 302A, B or different analytes. In some examples, the micropenetrators may be provided in sets with different heights. Thus when the analyte sensor 30 is in the deployed state, the analyte sensor 30 may collect data on analyte levels at various locations in the user. In some examples, one set of micropenetrators may facilitate sensing of analyte levels at a shallow destination (e.g. intradermal) while another set of micropenetrators may facilitate sensing at a deeper location (e.g. subcutaneous). The height of the micropenetrators may be selected such that the micropenetrators puncture the appropriate depth into the patient to reach the desired sensing locations.

Referring now to FIGS. 50-54, example embodiments of an access assemblies 46 are depicted. Each access assembly 46 may be used to deliver one or more agent into a patient as well as collect sensor data related to at least one analyte level of interest. Thus, a single assembly 46 may be applied instead of a first assembly for fluid delivery and a separate assembly for analyte sensing.

The access assembly 46 may include a main body 552. The main body 552 of the access assembly 46 may have a round (e.g. circular) foot print and may include a central region 500 and a peripheral region 502. The central region 500 may be a raised region of the main body 552 and the peripheral region 502 may surround the central region 500. The main body 552 may be injection molded. The central region 500 may be substantially planar in some examples and may include a base surface 570. The main body 552 may include a number of slots 508. The slots 508 may extend from a peripheral edge 510 of the main body 552 toward a center or midpoint of the main body 552. In the example embodiment, the slots 508 extend in a radial direction. The slots 508 may extend through the entirety of the peripheral region 502. The main body 552 may thus include a central region 500 which is circumscribed by a number of petal members 514 which are spaced apart via the slots 508.

The central region 500 may include a coupling 98 which may be defined on the base surface 570 of the central region 500. The coupling 98 may be as described in relation to FIGS. 41-46. In alternative embodiments, the coupling 98 may be a fitting (e.g. luer fitting). A connector 26 which may, for example, be attached to tubing 28 leading to an infusion device 18 may be releasably coupled to the access assembly 46. The connector 26 may be coupled to the access assembly 46 as described in relation to FIGS. 41-46. In the example embodiment, the base surface 570 of the central region 500 may include a set of conductive contacts 540. The contacts 540 may be in electrical communication with respective electrodes 302A, B of an analyte sensor 30 of the access assembly 46. The connector 26 may include conductive contacts on a surface of the connector 26 which abuts the base surface 570 when the connector 26 is coupled to the access assembly 46. Thus, when the connector 26 is coupled to the access assembly 46, the connector 26 may establish electrical communication with the analyte sensor 30 portion of the access assembly 46. An electrical conduit 542 may extend from each of the connector's 26 conductive contacts though the connector 26 and along the tubing 28. The infusion pump 18 may be in electrical communication with the conductive contacts of the connector 26 via the electrical conduits 542. No transmitter 32 may be used in such embodiments as the infusion device 18 may be in direct communication with the analyte sensor 30. In other embodiments, the connector 26 may include the transmitter 32. In still other embodiments, a separate transmitter 32 may be removably coupled (e.g. thread, clip, snap, adhere, etc.) to the access assembly 46. In some embodiments, the transmitter 32 may be provided as part of the access assembly 46 and may not be removable from the access assembly 46.

Referring now primarily to FIG. 50 and FIGS. 52-54, plan views of proximal faces 556 of example main bodies 552 are depicted. A proximal face 522 of a central region 500 of the main bodies 552 may include at least one stage projection 156 to which at least one access member 16 is coupled. The access member(s) 16 may include at least one microneedle on a sharp bearing body 74 in certain embodiments. Where microneedles are used, the microneedles may be any of the microneedles described herein. The stage projection(s) 156 may be any of the stage projections 156 described herein. A proximal face 522 of a central region 500 of the main bodies 552 may include at least one stage projection 358 upon which at least one set of electrodes 302A, B is disposed. The electrodes 302A, B may be silicon micropenetrators such as any of those described herein. Each electrode 302A, B may be included on a separate sharp bearing body 374 in such embodiments. Alternatively, the electrodes 302A, B may be conductive traces present on an insulative layer included on respective micropenetrators. In such embodiments, the micropenetrators on which the electrodes 302A, B are included may be formed on the same sharp bearing body 374.

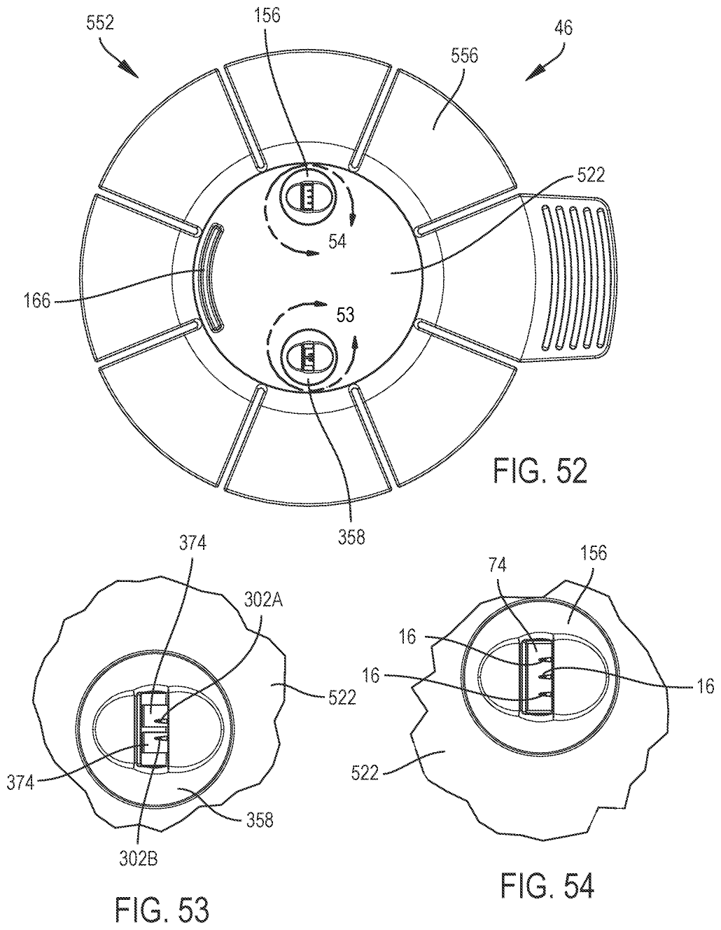

Each of the example access assemblies 46 shown in FIGS. 50 and 52 include the same stage projection 156 access member 16 arrangement and the same stage projection 358 electrode 302A, B arrangement. The placement of the stage projections 156, 358 differs between the embodiments. In the embodiment shown in FIG. 52, a rocker member 166 is included on the proximal face 522 of the central region 500. The stage projection 156, 358 are disposed on opposing sides of a mid-region of the proximal face 522 of the central region 500. The rocker member 166 may cause tilting of the central region 500 and access member(s) 16 as described in relation to FIGS. 35-26. The micropenetrators or electrodes 302A, B may also tilt in the same manner as the access member(s) 16. In the embodiment shown in FIG. 50, stage projection 358 has been moved to the location of the rocker member 166 and a rocker member 166 is absent. The stage projection 156 is centrally disposed on the proximal face 522 of the central region 500 (as described in relation to FIGS. 35-36). The position of stage projection 156 and stage projection 358 may be swapped in alternative embodiments. Each stage projection 156, 358 may be substantially the same height in various embodiments. The peripherally located stage projection 358 may act as a rocker member 166. As the access assembly 46 is transitioned to a deployed state, the micropenetrators and access member(s) 16 may puncture the skin 14. The peripherally located stage projection 358 may inhibit further displacement of a side of the central region 500 toward the skin 14 once it contacts the skin 14. An opposing side of the central region 500 may not be blocked from further displacement toward the skin 14 and the central region 500 may rock or tilt as the transition completes. This may cause tilting of the access member(s) 16 in the manner described in relation to FIGS. 35-36. The micropenetrators may similarly tilt within the skin 14.

As with various exemplary sets 12 and exemplary analyte sensors 30 described herein, example access assemblies 46 may include multiple sets of access members 16 and/or multiple sets of electrodes 302A, B. Each may, for example, be disposed on its own respective stage projection 156, 358. Where a plurality of access member 16 sets and/or a plurality of electrode 302A, B sets are included, the different access member 16 and or electrode 302A, B sets may all be substantially the same. Alternatively, they may include various differences as described elsewhere herein. For example, different sets of access members 16 may each include one or more access member(s) 16 and the height of the access member(s) 16 may differ between sets. Similarly, different sets of electrodes may differ in height. In some implementations, an access assembly 46 may include access member(s) 16 for intradermal and subcutaneous delivery (each may be a microneedle of suitable length). An example access assembly 46 may also include electrodes 302A, B for an analyte sensor 30 monitoring intradermal analyte levels and include electrodes 302A, B for an analyte sensor 30 monitoring subcutaneous analyte levels.

Still referring to FIGS. 50-54, adhesive may be included on at least a portion of the proximal face 556. Various adhesive bearing members 54 which may be coupled to a proximal face 556 of a main body 552 of an access assembly 46 are shown and described in relation to FIGS. 55-57. The adhesive may be a skin compatible adhesive and may serve to couple an access assembly 46 to a skin 14 surface at a desired site. Adhesive may be included on the peripheral region 502 of the main body 552 as well as a portion of a proximal face 522 of the central region 500.

The example access assembly 46 is depicted in a storage state in FIGS. 50-54. When the access assembly 46 is applied to a patient, the access assembly 46 may be placed on the skin 14 in the storage state. The adhesive on the proximal face 556 of the main body 552 may anchor the access assembly 46 to the skin 14. The main body 552 may be a deformable body which may transition from the storage state to a deployed state (either reversibly or irreversibly). As transition to a deployed state occurs, at least two adhesive bearing portions of the main body 552 may be spreadingly displaced with respect to one another so as to stretch or spread a skin surface anchored to the main body 552 via the adhesive. This may be desirable as the skin 14 may be rendered taught facilitating piercing of the skin 14 by the access member(s) 16 and electrodes 302A, B as the main body 552 transitions to the deployed state. The adhesive present on an adhesive member 54 (see, e.g., FIGS. 55-57) may aid in holding the access assembly 46 in the deployed state during use. No inserter assembly may be needed to apply the access assembly 46.

An example access assembly 46 may be applied to the skin 14 and a user may press down on the main body 552 to transition the access assembly 46 to the deployed state. This may avoid the need for a plurality of needle sticks each time an access assembly 46 is applied. This may make use of an access assembly 46 more attractive, particular for certain patient populations (e.g. those with juvenile diabetes). Example access assemblies 46 may obviate the need for a first inserter for an infusion set and a second inserter for an analyte sensor. Removal of any inserters from the application process may aid in defraying financial considerations of a patient which may dissuade some patients from opting to use an analyte sensor 30. With the omission of an inserter, financial burden associated with frequent site changes may be mitigated. This may help increase patient compliance with prescribed site change schedules or may allow for site changes to be performed more frequently (e.g. daily).

Referring now to FIGS. 55-57, a number of example adhesive members 54 are depicted on exemplary sets 12. The adhesive members 54 shown in FIGS. 55-57 may also be included on analyte sensors 30 or access assemblies 46 described elsewhere herein. As shown, a single adhesive member 54 is included for each of the example sets 12. In alternative embodiments, the adhesive member 54 may be broken into a plurality of individual adhesive members 54. This may facilitate use of different adhesives or perhaps leaving certain petal member 114 devoid of adhesive. As shown, each adhesive member 54 may include a plurality of slits 216 extending radially inward from a periphery of the adhesive member 54 so as to create petal portions which align with the petal members 114 of the main body 52. The adhesive member 54 may also include a central aperture 218 through which the access member(s) 16 of the set 12 may access a patient.

The shape and size of the central aperture 218 may play a role in helping to facilitate certain shallow deliveries. In various exemplary sets 12, it may be desirable that the central aperture 218 have a cross sectional area which is 60-100% the area of the footprint of the central region 100. It may also be desired that the central aperture 218 be shaped such that at least a portion of the adhesive member 54 is attached to a portion of a body 50 or proximal side 222 of the central region 100. In certain examples, the cross-sectional area of the central aperture 218 may be greater than 0.13 in². In certain examples, the cross-sectional area of the central aperture 218 may be in a range of 0.13 in² to 0.5 in² (e.g. about 0.3 in²) though may be outside of this range in various embodiments.

Additionally, it may be desired that the central aperture 218 be wider in certain directions compared to others. For instance, each access member 16 (e.g. one or more microneedle) may tend to dispense fluid in an ejection direction which extends from the outlet of the respective access member (e.g. along the axis of the lumen 68 of the access member 16). It may be desired that the central aperture 218 have a larger or increased width in a direction which aligns or substantially aligns with the ejection direction. For example, the greatest width (or at least a comparatively large width portion) of the central aperture 218 may be along a direction that is parallel to a plane that includes the ejection direction. Using a set 12 including one or more microneedle similar to that shown in FIG. 5, an increased width portion of the central aperture 218 may be aligned with the front to back (distal side 60 to back facing edge 66, may also be referred to herein as length) direction or line of symmetry of the microneedle. For example, the central aperture 218 could be obround or stadium shaped (see, e.g., FIG. 57) and be widest in a direction parallel to the front to back direction (or length dimension) of the microneedles. This may help to create a more diffuse shallow (e.g. intradermal) injection as opposed to a concentrated bleb at least for certain injections. This may, in turn, be desirable as it may help to expose the agent to more of the vasculature at the delivery destination and may help to facilitate absorption.

Referring now primarily to FIG. 55, the central aperture 218 may generally be a round (e.g. circular) aperture with the exception of a number of inwardly extending teeth or spokes 220 of adhesive member 54 material. In the example embodiment, the adhesive member 54 includes a central aperture 218 with four spokes 220 spaced at regular angular increments from one another. The number of spokes 220 may differ and the spacing of the spokes 220 may be irregular in certain examples. The spokes 220 may be disposed such that the central aperture 218 has a comparatively large width in a direction aligned with the ejection direction. Though the central aperture 218 may have a comparatively large width in this direction, this does not preclude other wide regions of equal, lesser, or perhaps even greater width. In the example shown, the central aperture 218 is about equal in width when measured in a direction perpendicular to the front to back direction of the microneedles. In certain examples, the spokes 220 may be the only portion of the adhesive member 54 which is adhered to the body 50 or proximal face 222 of the central region 100.

Referring now primarily to FIG. 56, in certain examples, the central aperture 218 may include notches 224 which extend outwardly from the periphery of the rest of the central aperture 218. The notches 218 may be included to widen the central aperture 218 where desired. Though rectangular notches 218 are included in the examples, the shape of the notches may differ in alternative embodiments. The notches 218 could be any suitable polygonal shape or could be round for example.

Referring now also to FIG. 45, a user may press upon the central region 100 of the set 12 to transition the set 12 to a delivery state. As example sets 12 are transitioned to the delivery state, the access members(s) 16 and stage projection 156 may displace toward the skin 14. A spreading displacement of petal members 114 of the set 12 may also occur. This spreading displacement may stretch skin 14 (see, e.g., FIG. 1) to which the set 12 is adhered and facilitate puncture of the skin 14 by any access member(s) 16 of the set 12 as they are displaced into the skin 14. The slits 216 in the adhesive member 216 may allow for the adhesive to accommodate the spreading displacement of the petal members 114 during the transition to the delivery state.

Initially when a set 12 is applied to the skin 14, adhesive at the peripheral region 102 of the set 12 may contact the skin 14. The set 12 may decrease in height as the transition to the delivery state occurs. As this occurs, adhesive on the adhesive member 54 inward of the peripheral region 102 may begin to contact and stick to the skin 14. Adhesive on the portion of the adhesive member 54 extending partially onto the proximal side 222 of the central region 100 (or alternatively a body 50 attached to the main body 52) may adhere to the skin 14 as the transition completes. The adherence of the adhesive to the skin 14 may maintain the set 12 in the delivery state and cause the skin 14 to move together with the set 12 as a unit. This may help to ensure that the access member(s) 16 remain at a desired location within the skin 14 as the set 12 is used.

Additionally, when the set 12 is in a delivery state, the stage projection 156 may press into and create a depression in the skin 14 when the set 12 is in the delivery state. The depressed skin 14 may tend to attempt restore against the stage projection 156 toward an undepressed position while the set 12 is worn. The adhesive on the adhesive member 54 in the vicinity of the stage projection 156 may help to encourage this. Thus, the skin pierced by the access member(s) 16 may tend to displace together with the access member(s) 16 and stage projection 156 even when these components move in a direction away from the skin 14. This may help to keep the access member(s) 16 firmly within the skin 14 as the set 12 is worn and help to inhibit leaks during delivery.

Referring now to FIGS. 58-63, a delivery assembly 24 or arrangement which may be included in a delivery device 18 (see, e.g., FIG. 1) is shown. The delivery assembly 24 depicted is an exemplary delivery assembly 24 and delivery devices 18 may include any of a variety of delivery arrangements 24. Certain delivery devices 18 may be syringe pumps. Certain delivery devices 18 may include peristatic pumping mechanisms (e.g. linear or finger type pumping mechanism or rotary peristaltic mechanism). Where a delivery device 18 delivers multiple agents to one or more set(s) 12, the infusion device 24 may include multiple of the delivery assembly 24 shown in FIGS. 58-63 each in communication with a separate agent reservoir 22.

In the example delivery assembly 24, an occluder assembly 232 may isolate a filled reservoir 22 from the delivery assembly 24. Opening of the occluder assembly 232 may allow fluid to flow into the remainder of the delivery assembly 24. In order to effectuate the delivery of fluid within the reservoir 22 to the user, a controller 20 (see, e.g., FIG. 1) included within a delivery device 18 may command energizing of a shape memory actuator 234, which may be anchored on one end using a shape memory actuator anchor 236. An opposing end of the shape memory actuator 234 may be coupled to a common connector 238 attached to a pump plunger 240A and reservoir valve assembly 242. Energizing of the shape memory actuator 234 may result in the activation of a pump 240 and the reservoir valve assembly 242. The reservoir valve assembly 242 may include a reservoir valve actuator 242A and a reservoir valve 242B. Activation of the reservoir valve assembly 242 may result in the downward displacement of the reservoir valve actuator 242A and the closing of the reservoir valve 242B, resulting in the effective isolation of the reservoir 22 from the delivery assembly 24. A membrane 244 may be included between a pump plunger 240A and a pump chamber 240B of the pump 105. The reservoir valve actuator 242A may press the membrane 244 against a valve seat of the reservoir valve 242B in order to close the reservoir valve assembly 242. Pump 240 and reservoir valve assembly 242 may be arranged and connected by the connector 238 whereby reservoir valve assembly 242 may close prior to the pump 240 pumping fluid. The activation of the pump 240 may result in the pump plunger 240A being displaced in a downward fashion into the pump chamber 240B leading to a displacement of the fluid (in the direction of arrow 246). The pump chamber 240B may be shaped to be substantially the same as the end of the pump plunger 240A in order to substantially empty the pump chamber 240B with each stroke of the pump 240.

A volume sensor valve assembly 248 may include a volume sensor valve actuator 248A and a volume sensor valve 248B. Referring also to FIG. 60, the volume sensor valve actuator 248A may be maintained in a closed position via a volume valve spring assembly 248C (e.g. acting against a spring anchor 250) that provides mechanical force to move the volume sensor valve actuator 248A against the volume sensor valve 248B to seal volume sensor valve 248B. The volume sensor valve actuator 248A may press a membrane 244 included in the cassette assembly 25 against a valve seat of the volume sensor valve 248B in order to close the volume sensor valve 248. When the pump 240 is activated, however, if the displaced fluid is of sufficient pressure to overcome the mechanical sealing force of the volume sensor valve assembly 248, displacement of the fluid may occur in the direction of arrow 252. This may result in the filling of a volume sensor chamber 256 included within a volume sensor assembly 258 (shown in FIG. 62). Through the use of a speaker assembly 260, port assembly 262, reference microphone 264, spring diaphragm 266, and variable volume microphone 268, the volume sensor assembly 258 may determine the volume of fluid within the volume sensor chamber 256. Operation of such a volume sensor assembly 258 may be as discussed in, for example, U.S. Pat. No. 8,491,570 issued Jul. 23, 2013 and entitled Infusion Pump Assembly (Attorney Docket No. G75) which is incorporated herein by reference in its entirety above. Other suitable dispensed volume sensors may be used in other embodiments.

Referring also to FIG. 62, a shape memory actuator 270 may be anchored (on a first end) to a shape memory actuator anchor 272. Additionally, the other end of the shape memory actuator 270 may be used to provide mechanical energy to a valve actuator 274, which may activate a measurement valve assembly 276. Once the volume of fluid included within the volume sensor chamber 256 is calculated, the shape memory actuator 270 may be energized, resulting in the activation of measurement valve assembly 276. The measurement valve assembly 270 may include a measurement valve actuator 276A and a measurement valve 276B. Once activated to lift the measurement valve actuator 276A from the measurement valve 276B, due to the mechanical energy asserted on the fluid within volume sensor chamber 256 by the spring diaphragm 266, the fluid within the volume sensor chamber 256 may be displaced (in the direction of arrow 278) through set 12 and into the patient. The measurement valve actuator 276A may then, by de-energizing the shape memory actuator and by action of the measurement valve spring assembly 276C (e.g. acting against spring anchor 280) press a membrane included in the cassette assembly 25 against a valve seat 276B in order to close the measurement valve 276B. In some embodiments, the membrane interfaces 244 included over the reservoir valve 242B, pump chamber 240B, volume sensor valve 648B, and the measurement valve 276B may be formed in a single piece of material having regions overlying each of these components.

As fluid is delivered to a set 12 or an access assembly 46, at least one characteristic related to the delivery may be monitored by at least one sensor of the delivery arrangement 24. The controller 20 of an infusion device 18 may analyze data from the one or more sensor to determine whether delivery is occurring in a desired manner. For example, a controller 20 may monitor data from at least one sensor in the delivery arrangement 24 to determine information related to the impedance to fluid delivery from an access member 16. Above the skin, there is very little impedance to deliver from an access member 16 as the access member 16 is in air. In the intradermal space, the impedance is relatively high and it is relatively difficult to drive fluid into the intradermal space. Delivery impedance may be relatively low into subcutaneous tissue compared to the intradermal tissue.

As fluid is delivered to a set 12, a pressure decay may occur as fluid is expelled from the set 12 and into a shallow delivery destination. Typically fluid may flow relatively slowly out of a set 12 and into a shallow delivery destination. The associated pressure decay may be relatively slow. If an access member 16 becomes dislodge or is no longer in the skin 14 fluid may more easily pass out of the access member 16 and flow rate out of the access member 16 may be greater than expected. Pressure decay may be comparatively rapid in such a scenario. If an access member 16 punctures beyond a shallow destination and into the subcutaneous space, pressure decay may also transpire more rapidly.

In some examples, data from at least one pressure sensor monitoring fluid pumped to the set 12 may be analyzed by a controller 20 to determine how quickly the pressure decay occurs after a volume of fluid has been delivered to the set 12. In the event that the pressure decays faster than a predefined threshold (e.g. the derivative of the pressure data exceeds a predefined value) it may be determined that access member(s) 16 may have displaced from a desired location. In some examples, the predefined threshold may be preprogrammed or may be calculated based on historical data from previous deliveries of agent from an infusion device 18. A controller 20 may monitor data from the at least one pressure sensor for changes in the speed of pressure decay as volumes of fluid are delivered through the set 12. In the event that pressure decay rate changes more than a certain threshold, a controller 20 may determine that an access member 16 has changed positions.

With regard to any determinations made by a controller 20 based on data related to access members 16 and/or analyte sensors 30 described herein, a controller 20 may generate an associated alert upon making such determinations. The alert may be displayed on a user interface of an infusion device 18. The alert may also include an audible (tone, series of tones, beep, beeps, etc.) or tactile alert (a vibratory motor may be activated) issued by an infusion device 18 or other component of the system 10. The controller 20 may also orchestrate communication of the alert to various components of a system 10. For example, the controller 20 may communicate the alert to at least one smart device (e.g. smart phone, smart watch, tablet, etc.) which may display the alert and/or issue an audible or tactile alert of its own. The controller 20 may also communicate the alert to the cloud 38. The controller 20 may also determine an appropriate user intervention (e.g. new set 12, or analyte sensor(s) 30, or access assembly 46 needed) and request the user perform the intervention via a user interface of the system 10.

Additionally, pressure decay may be slower than expected in the event of an occlusion. A controller 20 may similarly analyze data from at least one pressure sensor to monitor for pressure decay characteristics (e.g. rate of decay, change in rate of decay compared to previous data) indicative of an occlusion. In the event that the pressure decays slower than a predefined threshold, a controller 20 may determine that an occlusion is present. Similarly if a change in rate of pressure decay to a rate more than a predefined amount slower than previous data is observed, a controller 20 may determine an occlusion is present.

In certain examples and still referring to FIGS. 58-63, data from at least one volume sensor assembly 258 may be checked multiple times as a volume of fluid is delivered from the infusion device 18 to the set 12. A controller 20 may determine change in depth of an access member 16 or an occlusion has occurred based on data from the at least one volume sensor assembly 258. In the event that a difference in volumes measured by the at least one volume sensor assembly 258 over a predetermined period of time is larger than a predefined amount or the rate of change is too fast, a controller 20 may determine that an access member 16 has changed position. Similarly, if the change in volume is less than a predefined amount or the rate of change is too slow, a controller 20 may determine that an occlusion is present. Changes in volume or rates of change in volume may also be compared to previous data to determine whether the delivery impedance differs from previous deliveries.

An infusion device 18 of the system 10 may deliver independently to at least one long access member 16 or at least one short access member 16 included in, for example, a set 12. In systems 10 where agent may be delivered through two or more selected access member(s) 16 of different lengths, a controller 20 may compare delivery data from deliveries to at least two of the different length access members 16. In certain examples, a set 12 or access assembly 46 may include at least one long access member 16 which communicates with a subcutaneous delivery destination and a short access member 16 which communicates with a shallow delivery destination. As mentioned elsewhere herein each may microneedles of different lengths.

Typically, a displacement of the at least one short access member 16 may be associated with a like displacement of the at least one long access member 16. The at least one short access member 16 and at least one long access member 16 may be placed as close to one another as is practicable to help ensure tight a correlation of displacement of the at least one short and at least one long access member 16. In the event that the at least one short access member 16 is displaced such that it is out of the skin 14, the at least one long access member 16 may generally displace a like amount in the same direction. This displacement of the long access member 16 may be sufficient for the long access member 16 to be positioned at an intradermal delivery destination. The height of the at least one long access member 16 and/or position of any flow lumen(s) 68 or channel(s) 70 in the at least one long access member 16 may be selected to help ensure this occurs. In the event that the at least one short access member 16 has punctured into subcutaneous space, the at least one long access member 16 may still be within the subcutaneous space.

When data indicative of the delivery impedance (e.g. pressure decay, volume dispensed as sensed by a volume sensing assembly 258) from the at least one short access member indicates the at least one short access member 16 has changed depth, the controller 20 may analyze data from a delivery to the at least one long access member 16. In some embodiments, the controller 20 may orchestrate a delivery to the at least one long access member 16 to collect data. In some embodiments, the at least one long access member 16 may not typically be used for delivery. In such examples, the at least one long access member 16 may be testing access member 16 used to collect data when desired.

Upon analysis of delivery data related to the at least one long access member 16 indicating delivery impedance characteristics which would be expected from an intradermal delivery, the controller 20 may determine that the at least one short access member 16 has become dislodged from the skin 14. The controller 20 may halt delivery to the at least one short access member 16 and deliver exclusively to the at least one long access member 16 so that therapy may be continued. Depending on the embodiment, if a controller 20 determines (in any manner describe herein) that one or more access member 16 is out of the skin 14, the controller 20 may halt delivery to the access member(s) 16. Where a controller 20 determines at least one access member 16 is still capable of delivering fluid to the patient, the controller 20 may adjust delivery such that fluid is only delivered to access member(s) 16 still in the patient. The controller 20 may generate an alert conveying that this has occurred. In some embodiments, the controller 20 may halt all delivery or prompt a user to confirm desire to divert deliveries to certain of the access member(s) 16.

In the event that delivery data related to the at least one long access member 16 indicates delivery impedance increased and subsequently decreased, it may be determined by the controller 20 that it is likely that the at least one long and at least one short access member 16 have moved out of the skin 14. The controller 20 may generate an alert to this effect for display on a user interface of the infusion device 18 and/or for communication to another component of the system 10 (e.g. for display on a smart phone and/or smart watch). The controller 20 may halt delivery and may, for example, generate an indication that the set 12 should be swapped out for a new set 12.

When analysis of delivery data related to the at least one short access member 16 indicates delivery impedance characteristics which are in line with previous deliveries form the at least one long access member 16, the controller 20 may determine the at least one short access member 16 has displaced deeper.

In still other embodiments, access members 16 to which fluid may be independently delivered may be included in at least three different heights on a set 12. The shortest access member(s) 16 may have a height which generally inhibits passage into the subcutaneous space. In such embodiments, there may be at least one long access member 16 and at least one access member 16 of intermediate height. The intermediate height access member(s) 16 may have a height sufficient to deliver to an intradermal delivery destination. The long access member(s) 16 may have a height sufficient to deliver to a subcutaneous delivery destination.

In the event that sensor data related to delivery of a volume of fluid to the shortest access member(s) 16 indicate a high delivery impedance is present, the intermediate length and long access members 16 ought to be in the skin 14. Given high delivery impedance from the shortest access member(s) 16, when data related to the impedance to flow from the intermediate access member(s) 16 indicates a drop in impedance, it may be determined by a controller 20 that the intermediate access member(s) 16 are in communication with the subcutaneous space. In the event that data related to delivery of volumes of fluid to the shortest and intermediate access member(s) 16 indicates low impedance, it may be determined by a controller 20 that these access member(s) 16 have displaced out of the skin 14. The controller 20 may generate an alert communicating such determinations for display on a user interface of the infusion device 18 and/or for communication to another component of the system 10.

The controller 20 may make delivery calculations based at least in part upon analyte sensor 30 data and expected agent absorption profiles. As the absorption profile for the agent may be dependent on the depth of the delivery destination, the controller 20 may adjust delivery calculations based on the determined depth of the access member(s) 16 using on the impedance related data. For example, the controller 20 may adjust the timing of a delivery in the event the controller determines the agent will be absorbed faster or slower. Other calculations may also be adjusted base on the determined depth of the access member(s) 16. Using the example of insulin, insulin on board (I.O.B.) calculations or duration of insulin action (D.I.A.) calculations may be adjusted. Similar agent on board or duration of agent action determinations for other agents may be adjusted where the agent is an agent other than insulin.

In analyte sensors 30 and access assemblies 46 having multiple sets of electrodes 302A, B sensing analyte levels at different depths, data from the each set 302A, B of electrodes may be compared. In the event that a pair of electrodes 302A, B of an analyte sensor 30 are in air, the reading from the analyte sensor 30 should indicate that the analyte sensor 30 is not monitoring the appropriate location. Additionally, analyte sensors 30 monitoring a highly vascularized space (e.g. shallow or intradermal location) and a less vascularized space (e.g. subcutaneous space) to determine blood analyte levels should collect data which differs in a predictable and related manner.

Using diabetes as a non-limiting example, interstitial glucose levels in the subcutaneous space may lag behind those in the blood a greater amount than interstitial glucose levels in the intradermal space. Thus, an analyte sensor 30 monitoring, for example, a subcutaneous space would see a response to blood glucose level changes which is delayed or time shifted with relation to a response to changes in blood glucose levels sensed by an analyte sensor 30 monitoring the intradermal space.

A delivery of insulin or glucagon from an infusion device 18 (or intake of carbohydrates) should alter blood glucose levels and this alteration would be most clearly seen first by the analyte sensor 30 monitoring the intradermal space. Times at which a delivery of agent from an infusion device 18 and the volume of agent delivered may be known. In some embodiments, timing of carbohydrate consumption and amount of carbohydrate consumed may also be input by a user into certain systems 10. Additionally, an expected adjustment of the blood glucose level should be engendered by the agent delivery (or carbohydrate consumption). The response to blood glucose changes in the wake of, for instance, an agent delivery observed in the subcutaneous tissue should lag that observed in the intradermal tissue. Data from a shallow and deeper analyte sensor 30 should generally track each other in a predictable way over the life time of the analyte sensors 30. An expected relationship between data from a shallow analyte sensor 30 and a deeper analyte sensor 30 may be determined (e.g. by a controller 20) based on data collected from the analyte sensors 30. For example, an expected time shift (or expected time shift window or range) between data from each of the analyte sensors 30 may be determined. This relationship may be updated periodically over the life of the analyte sensors 30 and may be initially determined during or shortly after a warm up period of the analyte sensors 30. In some examples, the expected relationship may be initialized to a predefined anticipated relationship for the patient (e.g. from data collected from previous analyte sensor 30 usage) when new analyte sensors 30 are placed.

In some examples, the controller 20 may analyze analyte sensor 30 data to determine analyte level trend information which may be displayed via a user interface to a user. For example, current data may be compared to previous data to determine a trend in analyte level. For example, a controller 20 may generate a message or other indication showing that blood glucose levels are trending downward (or downward sharply), upward (or upward sharply), or remaining generally level. In some examples, the controller 20 may determine trend information based on data from only one of the analyte sensors 30 or determine trend information by according a higher weighting to data from one of the analyte sensors 30. In the event that a deeper analyte sensor 30, for example, suggests a trend which differs from the trend sensed by an intradermal analyte sensor 30, the data from the intradermal analyte sensor 30 may be used or at least be more heavily weighted in determining a current trend for display. In some examples, the difference between data from each of the analyte sensors 30, derivatives of data, or derivatives of the difference between the data may also or instead be used to determine blood analyte level trend information.

In the event that a shallow analyte sensor 30 begins to output data indicative of the analyte sensor 30 being in air, data from a deeper analyte sensor 30 may be checked by a controller 20. In the event that the deeper analyte sensor 30 begins to output data indicative of the deeper analyte sensor 30 also being in air, a controller 20 may determine that the analyte sensors 30 have fallen out of the skin. In the event that the data from the deeper analyte sensor 30 is recording a response to blood glucose changes (e.g. due to agent administration) which conforms to that previously typical of the shallow analyte sensor 30, the controller 20 may determine that the shallow analyte sensor 30 has displaced out of the skin 14.

Additionally, data from a shallow analyte sensor 30 and a deeper analyte sensor 30 may be compared to determine a particular analyte sensor 30 is beginning to display abnormal behavior (e.g. has or is beginning to drop out). Historical data may be used to assist in such determination. In the event, for example, a first of the analyte sensors 30 is diverging from a typical or expected response relationship to changes in blood analyte levels (e.g. due to agent deliveries or user actions such as eating) while the second of the analyte sensors 30 has not diverged outside of some predefined threshold, the first of the analyte sensors 30 may be beginning to drop out, for example. Alternatively or additionally, if a typical relationship (e.g. determined from historical analyte sensor 30 data) between the data from the first and second analyte sensors 30 begins to deteriorate, this may be indicative of a dropout issue or developing dropout issue with one of the analyte sensors 30. The controller 20 may determine that a drop out event is occurring for the relevant analyte sensor 30 based on the comparisons of analyte sensor 30 data described above. The controller 20 may convey an alert to a user interface of the infusion device 18 and/or communicate an alert for display on another component of the system 10. This may help prevent a user from acting on data from an analyte sensor 30 which is beginning to drop out.

In some examples, if the expected relationship between data from the analyte sensors 30 has deteriorated beyond some threshold, the controller 20 may analyze analyte sensor 30 data to perform various troubleshooting. For example, if the expected relationship is no longer present, the controller 20 may check to see if data from one of the analyte sensors 30 is indicative that the analyte sensor is in air 30. If the shallow analyte sensor 30 and deeper analyte sensor 30 report data which does not display time shifted sensed analyte level changes, a controller 20 may determine the shallow analyte sensor 30 may have changed depths to a deeper (e.g. subcutaneous) location. That is, if the analyte sensors 30 report roughly the same analyte level at the same time and/or that the analyte levels are changing at roughly the same rate over the same time period, the controller 20 may determine each analyte sensor 30 may be monitoring the same location in a patient.

In some embodiments, data collected in relation to any puncturing bodies regardless of purpose may be used in conjunction by a controller 20 to make various determinations about the location or status of the puncturing bodies. For example, analyte sensor 30 data and data related to delivery impedance from access member(s) 16 may utilized by a controller 20 to help determine status or location of certain of the puncturing bodies. For example, if a controller 20 makes a determination about an access member 16 or sensor as described elsewhere herein data related to another puncturing body may be checked. The additional data may help to confirm or verify the determination. In some embodiments, no alert may be generated until the additional data is analyzed. Data related to other access members 16 or data from other analyte sensors 30 may be used to assist in determining or verifying that a particular puncturing body has changed depths, has developed an occlusion, or in the case of an analyte sensor, that a drop out issue is present, for example.

Using the example of an access assembly 46 (see, e.g., FIG. 3) all puncturing bodies on the access assembly 46 may be relatively close to one another. In the event that data related to all access members 16 of an access assembly 46 indicates a low delivery impedance and analyte sensor 30 data indicates the analyte sensor 30 may be in air, it may be determined the access assembly 46 has fallen off or was removed. In the event that data related to all access members 16 indicated they are in an expected location and all but one of the analyte sensors 30 is outputting data within predefined expectations, it may be unlikely that the non-conforming analyte sensor 30 has changed depths. The controller 20 may thus surmise the non-conforming analyte sensor 30 may be dropping out.

If data indicative of the delivery impedance from an access member 16 indicates a different than expected impedance, the controller 20 may analyze analyte sensor 30 data. If data from the analyte sensor 30 indicates analyte level changes in response to agent delivery through the access member 16 do not conform to expectations, the controller 20 may determine the access member 16 has changed depths (e.g. subcutaneous as opposed to intradermal if slower than expected or vice versa if faster). Where multiple analyte sensors 30 monitoring different depths are present, data from each analyte sensor 30 may be checked. If the response to administration of a volume of agent takes longer than expected to change analyte levels detected by all analyte sensors 30, it may be determined that the access member 16 has displaced to a deeper delivery destination, for example. If data from a deeper analyte sensor 30 indicates a shorter lag time in sensed analyte level changes (e.g. after agent deliveries) and delivery impedance related data from at least one longer access member 16 indicates a higher than expected impedance, a controller 20 may determine that the deeper analyte sensor 30 and longer access member(s) 16 have changed depths (e.g. from a subcutaneous to an intradermal location). When this is observed, a controller 20 may check data from a shorter analyte sensor 30 and at least one shorter access member 16 to verify they have not displaced out of the skin 14. Additionally, if a controller 20 notes that a shallow analyte sensor 30 is providing data indicative of the analyte sensor 30 being in air and delivery impedance data related to at least one short access member 16 indicates the short access member(s) 16 may be in air, the controller 20 may check data from a deeper analyte sensor 30 and/or or longer access member(s) 16. The controller 20 may verify a change in depth of the deeper analyte sensor 30 and/or or longer access member(s) 16 is reflected in the relevant data before generating an alert.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. A medical agent administration device comprising:
a main body including a central region and a peripheral region having a plurality of petal members extending outwardly from the central region;
at least one coupling on a first face of the central region; and
at least one sharp bearing body on a second face of the central region opposite the first, each in fluid communication with a respective one of the at least one coupling.

2. The medical agent administration device of claim 1, wherein the central region is raised with respect to the peripheral region.

3. The medical agent administration device of claim 1, wherein the at least one coupling includes fluid transfer connector receivers each having a ramped face and a ledge face.

4. The medical agent administration device of claim 1, wherein the at least one coupling includes at least one guide for a fluid transfer connector.

5. The medical agent administration device of claim 1, wherein each of the at least one coupling includes a luer fitting.

6. The medical agent administration device of claim 1, wherein the second face of the central region includes at least one rocker member.

7. The medical agent administration device of claim 1, wherein each of the at least one sharp bearing body is coupled to a respective one of an at least one stage projection on the second face in an injection molding operation.

8. The medical agent administration device of claim 1, wherein the device includes a septum sealing a passage in fluid communication with the at least one sharp bearing body.

9. The medical agent administration device of claim 1, wherein the second face of the central region is at least partially covered with an adhesive bearing member.

10. The medical agent administration device of claim 1, wherein each of the at least one sharp bearing body includes at least one microneedle.

11. The medical agent administration device of claim 1, wherein a first of the at least one sharp bearing body includes at least one first microneedle having a first height and a second of the at least one sharp bearing body includes at least one second microneedle having a second height different from the first height.

12. The medical agent administration device of claim 11, wherein the first height is selected to place the at least one first microneedle in an intradermal delivery destination and the second height is selected to place the at least one second microneedle in a deeper delivery destination when the device is in use.

13. The medical agent administration device of claim 1, wherein each of the at least one sharp bearing body is in fluid communication with the respective one of the at least one coupling only.

14. An analyte sensor device comprising:
a main body including a central region and a peripheral region having a plurality of petal members extending outwardly from the central region;
at least one sharp bearing body on a first face of the central region, each of the at least one sharp bearing body including at least one electrode, the device including at least one first electrode and at least one second electrode which is associated with analyte sensing chemistry.

15. The device of claim 14, wherein each of the at least one electrode communicates with a conductive trace extending to an opposing side of the central region.

16. The device of claim 14, wherein an opposing face of the central region opposite the first face includes at least one coupling member for coupling the device to a transmitter.

17. The device of claim 14, wherein each of the at least one first electrode and each of the at least one second electrode are micropenetrators.

18. The device of claim 14, wherein the analyte sensor device is a glucose sensor.

19. A medical agent administration system comprising:
an infusion device including a delivery assembly including at least one sensor and at least one pumping arrangement;
a set in fluid communication with the delivery device, the set including at least one intradermal access member; and
a controller configured to govern operation of the at least one pumping arrangement, the controller in data communication with the at least one sensor and configured to analyze data from the at least one sensor and determine a change in depth of the at least one access member has occurred based on the data from the at least one sensor.

20. The system of claim 19, wherein the at least one sensor is configured to generate a data signal which varies in relation to a delivery impedance from the at least one access member.
